SEQUENCE LISTING (61 sequences)

SEQ ID NO: 1 - protein, 383 aa, Homo sapiens
MGLGPVFLLLAGIFPFAPPGAAAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 2 - protein, 383 aa, Homo sapiens [sequence omitted]

SEQ ID NO: 3 - protein, 93 aa, Homo sapiens
VPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 4 - protein, 93 aa, Homo sapiens
VPPMVNVTCSEVSEGNITVTCRASSFYPRNITLTWRQDGVSLSHNTQQWGDVLPDGNGTYQTWVATRIRQGEEQRFTCYMEHSGNHGTHPVPS

SEQ ID NO: 5 - DNA, 1365 bp, Homo sapiens
cactgcttgagccgctgagagggtggcgacgtcggggccatggggctgggcccggtcttcctgcttctggctggcatcttcccttttgcacctccgggagctgctgctgagccccacagtcttcgttataacctcacggtgctgtcctgggatggatctgtgcagtcagggtttctcactgaggtacatctggatggtcagcccttcctgcgctgtgacaggcagaaatgcagggcaaagccccagggacagtgggcagaagatgtcctgggaaataagacatgggacagagagaccagagacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactaaggaatggacaatgccccagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaacagtgccccccatggtgaatgtcacccgcagcgaggcctcagagggcaacattaccgtgacatgcagggcttctggcttctatccctggaatatcacactgagctggcgtcaggatggggtatctttgagccacgacacccagcagtggggggatgtcctgcctgatgggaatggaacctaccagacctgggtggccaccaggatttgccaaggagaggagcagaggttcacctgctacatggaacacagcgggaatcacagcactcaccctgtgccctctgggaaagtgctggtgcttcagagtcattggcagacattccatgtttctgctgttgctgctgctgctatttttgttattattattttctatgtccgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcgcctagactctacagccaggcagctgggattcaattccctgcctggatctcacgagcactttccctcttggtgcctcagtttcctgacctatgaaacagagaaaataaaagcacttatttattgttgttggaggctgcaaaatgttagtagatatgaggcgtttgcagctgtaccatatt

SEQ ID NO: 6 - DNA, 2380 bp, Homo sapiens
gggccatggggctgggccgggtcctgctgtttctggccgtcgccttcccttttgcacccccggcagccgccgctgagccccacagtcttcgttacaacctcatggtgctgtcccaggatgaatctgtgcagtcagggtttctcgctgagggacatctggatggtcagcccttcctgcgctatgacaggcagaaacgcagggcaaagccccagggacagtgggcagaagatgtcctgggagctaagacctgggacacagagaccgaggacttgacagagaatgggcaagacctcaggaggaccctgactcatatcaaggaccagaaaggaggcttgcattccctccaggagattagggtctgtgagatccatgaagacagcagcaccaggggctcccggcatttctactacgatggggagctcttcctctcccaaaacctggagactcaagaatcgacagtgccccagtcctccagagctcagaccttggctatgaacgtcacaaatttctggaaggaagatgccatgaagaccaagacacactatcgcgctatgcaggcagactgcctgcagaaactacagcgatatctgaaatccggggtggccatcaggagaacagtgccccccatggtgaatgtcacctgcagcgaggtctcagagggcaacatcaccgtgacatgcagggcttccagcttctatccccggaatatcacactgacctggcgtcaggatggggtatctttgagccacaacacccagcagtggggggatgtcctgcctgatgggaatggaacctaccagacctgggtggccaccaggattcgccaaggagaggagcagaggttcacctgctacatggaacacagcgggaatcacggcactcaccctgtgccctctgggaaggtgctggtgcttcagagtcaacggacagactttccatatgtttctgctgctatgccatgttttgttattattattattctctgtgtcccttgttgcaagaagaaaacatcagcggcagagggtccagagcttgtgagcctgcaggtcctggatcaacacccagttgggacaggagaccacagggatgcagcacagctgggatttcagcctctgatgtcagctactgggtccactggttccactgagggcgcctagactctacagccaggcggccaggattcaactccctgcctggatctcaccagcactttccctctgtttcctgacctatgaaacagaaaataacatcacttatttattgttgttggatgctgcaaagtgttagtaggtatgaggtgtttgctgctctgccacgtagagagccagcaaagggatcatgaccaactcaacattccattggaggctatatgatcaaacagcaaattgtttatcatgaatgcaggatgtgggcaaactcacgactgctcctgccaacagaaggtttgctgagggcattcactccatggtgctcattggagttatctactgggtcatctagagcctattgtttgaggaatgcagtcttacaagcctactctggacccagcagctgactccttcttccacccctcttcttgctatctcctataccaataaatacgaagggctgtggaagatcagagcccttgttcacgagaagcaagaagccccctgaccccttgttccaaatatactcttttgtctttctctttattcccacgttcgccctttgttcagtccaatacagggttgtggggcccttaacagtgccatattaattggtatcattatttctgttgtttttgtttttgtttttgtttttgtttttgagacagagtctcactcgtcacccaggctgcagttcactggtgtgatctcagctcactgcaacctctgcctcccaggttcaagcacttctcgtacctcagactcccgatagctgggattacagacaggcaccaccacacccagctaatttttgtattttttgtagagacggggtttcgccaagttgaccagcccagtttcaaactcctgacctcaggtgatctgcctgccttggcatcccaaagtgctgggattacaagaatgagccaccgtgcctggcctattttattatattgtaatatattttattatattagccaccatgcctgtcctattttcttatgttttaatatattttaatatattacatgtgcagtaattagattatcatgggtgaactttatgagtgagtatcttggtgatgactcctcctgaccagcccaggaccagctttcttgtcaccttgaggtcccctcgccccgtcacaccgttatcgattactctgtgtctactattatgtgtgcataatttataccgtaaatgtttactctttaaataaaaaaaaaaaaaaa

SEQ ID NO: 7 - protein, 216 aa, Homo sapiens
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 8 - DNA, 1606 bp, Homo sapiens
actaagtatctccactttcaattctagatcaggaactgaggacatatctaaattttctagttttatagaaggcttttatccacaagaatcaagatcttccctctctgagcaggaatcctttgtgcattgaagactttagattcctctctgcggtagacgtgcacttataagtatttgatggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatctccattttttttctgctgcttcatcgctgtagccatgggaatccgtttcattattatggtaacaatatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgtaaagatgatcaaccatctcaataaaagccaggaacagagaagagattacaccagcggtaacactgccaactgagactaaaggaaacaaacaaaaacaggacaaaatgaccaaagactgtcagatttcttagactccacaggaccaaaccatagaacaatttcactgcaaacatgcatgattctccaagacaaaagaagagagatcctaaaggcaattcagatatccccaaggctgcctctcccaccacaagcccagagtggatgggctgggggaggggtgctgttttaatttctaaaggtaggaccaacacccaggggatcagtgaaggaagagaaggccagcagatcactgagagtgcaaccccaccctccacaggaaattgcctcatgggcagggccacagcagagagacacagcatgggcagtgccttccctgcctgtgggggtcatgctgccacttttaatgggtcctccacccaacggggtcagggaggtggtgctgccccagtgggccatgattatcttaaaggcattattctccagccttaagtaagatcttaggacgtttcctttgctatgatttgtacttgcttgagtcccatgactgtttctcttcctctctttcttccttttggaatagtaatatccatcctatgtttgtcccactattgtattttggaagcacataacttgtttggtttcacaggttcacagttaagaaggaattttgcctctgaataaatagaatcttgagtctcatgcaaaaaaaaaaaaaaaaaa

SEQ ID NO: 9 - protein, 219 aa, Mus musculus
MSKCHNYDLKPAKWDTSQEQQKQRLALTTSQPGENGIIRGRYPIEKLKISPMFVVRVLAIALAIRFTLNTLMWLAIFKETFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 10 - protein, 215 aa, Rattus norvegicus
MSKCHNYDLKPAKWDTSQEHQKQRSALPTSRPGENGIIRRRSSIEELKISPLFVVRVLVAAMTIRFTVITLTWLAVFITLLCNKEVSVSSREGYCGPCPNDWICHRNNCYQFFNENKAWNQSQASCLSQNSSLLKIYSKEEQDFLKLVKSYHWMGLVQSPANGSWQWEDGSSLSPNELTLVKTPSGTCAVYGSSFKAYTEDCSNPNTYICMKRAV

SEQ ID NO: 11 - protein, 216 aa, Macaca mulatta
MGWIRGRRPRHNLEMSEFHNYKLGLAKSDFSTRCQKQRCPVIKSKCRENASPLFFCCFIAVAMGIRFIIMVTIWSAVFLNSLFNQEVQIPLTESYCGPCPKNWICYKNNCYQFFNESKNWYESQASCMSQNASLLKVYSKEDQDLLKLVKSYHWMGLVHIPTNGSWQWEDGSILSPNLLTIIEMQKGDCALYASSFKGYIENCSIPNTYICMQRTV

SEQ ID NO: 12 - protein, 216 aa, Macaca fascicularis; identical to SEQ ID NO: 11

SEQ ID NO: 13 - protein, 216 aa, Callithrix jacchus
MGWIRGRRPRHSLAMSEFRNYNLELAKGDFSTRWQKQRCPVIKSKCRENFTPLFFCCFIAVAMGIRFIVMVTIWTAVFLNSLFNQEVQVPLIESYCGPCPKNWICHRNNCYQFFNENKNWYESQASCMSQNASLLKVYSKAEQDFLKLVKSYHWMGLVYIPTNGSWQWEDGTILSPNLLTIIEMQKGDCAVYASSFKGYTENCSTPNTYICMQKTV

SEQ ID NO: 14 - protein, 216 aa, Pan troglodytes
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFNESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 15 - protein, 244 aa, Homo sapiens
MAAAASPAFLLCLPLLHLLSGWSRAGWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGTTQPKAMATTLSPWSLLIIFLCFILAGR

SEQ ID NO: 16 - protein, 246 aa, Homo sapiens
MAAAAATKILLCLPLLLLLSGWSRAGRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMSSGTTQLRATATTLILCCLLIILPCFILPGI

SEQ ID NO: 17 - protein, 244 aa, Homo sapiens
MAAAASPAILPRLAILPYLLFDWSGTGRADAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPGLAQPKAIATTLSPWSFLIILCFILPGI

SEQ ID NO: 18 - protein, 227 aa, Homo sapiens
MRRISLTSSPVRLLLFLLLLLIALEIMYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPDRWIILGAFILLVLMGIVLICVWWQNGEWQAGLWPLRTS

SEQ ID NO: 19 - protein, 334 aa, Homo sapiens
MAAAASPAFLLRLPLLLLLSSWCRTGLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSGTAQPRATATTLILCCLLIMCLLICSRHSLTQSHGHHPQSLQPPPHPPLLHPTWLLRRVLWSDSYQIAKRPLSGGHVTRVTLPIIGDDSHSLPCPLALYTINNGAARYSEPLQVSIS

SEQ ID NO: 20 - protein, 246 aa, Homo sapiens
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 21 - protein, 230 aa, Human cytomegalovirus
MERRRGTVPLGWVFFVLCLSASSSCAVDLGSKSSNSTCRLNVTELASIHPGETWTLHGMCISICYYENVTEDEIIGVAFTWQHNESVVDLWLYQNDTVIRNFSDITTNILQDGLKMRTVPVTKLYTSRMVTNLTVGRYDCLRCENGTTKIIERLYVRLGSLYPRPPGSGLAKHPSVSADEELSATLARDIVLVSAITLFFFLLALRIPQRLCQRLRIRLPHRYQRLRTED

SEQ ID NO: 22 - protein, 306 aa, Human cytomegalovirus
MRIEWVWWLFGYFVSSVGSERSLSYRYHLESNSSTNVVCNGNISVFVNGTLGVRYNITVGISSSLLIGHLTIQVLESWFTPWVQNKSYNKQPLGDTETLYNIDSENIHRVSQYFHTRWIKSLQENHTCDLTNSTPTYTYQVNVNNTNYLTLTSSGWQDRLNYTVINSTHFNLTESNITSIQKYLNTTCIERLRNYTLESVYTTTVPQNITTSQHATTTMHTIPPNTITIQNTTQSHTVQTPSFNDTHNVTKHTLNISYVLSQKTNNTTSPWIYAIPMGATATIGAGLYIGKHFTPVKFVYEVWRGQ

SEQ ID NO: 23 - protein, 430 aa, Human herpesvirus 7
MWTILLFCVPVIYGELYPDFCPLAVVDFDVNATVDDLLLFDISLSKQCSDDKIRHSAVAAMTDNAFFFGNSETQIETDFGKYLAFNCYQVFSTLNHFLFKNFKKTKGLMKRYDKLCLDVESYIHIQIICSPFKSFIRLRRMNETGISPRILETTFYLQNKRNSTWVAIKNYLGEDDPFTYRIWHTLTHAKNFLINSCENDFNQLFFWQRKYLSLAKTFEATFKQGFNPMIEQRNEQRYRTNNIDCSFSKFRQNGVKVAVCKYTGWGVSGFGSLEVLQKIKSPFGEEWKRVGFNSTGAFTPLYGSDVLWGLIFLRVEMTTYVCTCTNKNTGTQIQVTLPDVDLDLLDSEKTSSNVFVDMLCYTLIAILFLAFVTAVVLLGVSCLDGVQKVLTWPLQHIQKEPVSEKIINLTNLMFGQEPLPKKESLKQQCL

SEQ ID NO: 24 - protein, 7 aa, Homo sapiens
RSEASEG

SEQ ID NO: 25 - protein, 5 aa, Homo sapiens
RQDGV

SEQ ID NO: 26 - protein, 5 aa, Homo sapiens
LPDGN

SEQ ID NO: 27 - protein, 6 aa, Homo sapiens
QGEEQR

SEQ ID NO: 28 - protein, 6 aa, Homo sapiens
RGEEQR

SEQ ID NO: 29 - protein, 7 aa, Homo sapiens
CSEVSEG

SEQ ID NO: 30 - protein, 5 aa, Homo sapiens
RQDGV

SEQ ID NO: 31 - protein, 5 aa, Homo sapiens
LPDGN

SEQ ID NO: 32 - protein, 7 aa, Homo sapiens
RQGEEQR

SEQ ID NO: 33 - protein, 7 aa, Homo sapiens
  VARIANT (1)..(1): Xaa is Arg or Cys
  VARIANT (3)..(3): Xaa is Glu or Lys
  VARIANT (4)..(4): Xaa is Ala or Val
XSXXSEG

SEQ ID NO: 34 - protein, 5 aa, Homo sapiens
  VARIANT (5)..(5): Xaa is Val or Leu
RQDGX

SEQ ID NO: 35 - protein, 7 aa, Homo sapiens
  VARIANT (1)..(1): Xaa is Arg or Cys
  VARIANT (2)..(2): Xaa is Gln, Arg, or Glu
  VARIANT (7)..(7): Xaa is Arg, Ser, or Lys
XXGEEQX

SEQ ID NO: 36 - protein, 5 aa, Homo sapiens
LPDGN

SEQ ID NO: 37 - protein, 112 aa, Artificial Sequence
  light chain variable region of antibody 1F5
DIVLTQSPASLAVSLGQRATISCRASKSVSTSGYSYMHWYQQKPGQPPKLLIYRASNLESGVPARFSGSGSGTDFTLNIHPVEEEDAATYYCQHSRELPLTFGAGTKLELKR

SEQ ID NO: 38 - protein, 117 aa, Artificial Sequence
  heavy chain variable region of antibody 1F5
QIQLVQSGPELKKPGETVKISCKASGYTFTDYSVHWVKQAPGKGLKWMGWINTETGEPTYADDFKGRFAFSLETSASTAYLQINNLKNEDTATYFCARAGGNAFAYWGQGTLVTVSA

SEQ ID NO: 39 - protein, 113 aa, Artificial Sequence
  light chain variable region of antibody 8C7
DIVMTQAAPSVPVTPGESVSISCRSSKSLLQSNGNTFLYWFMQRPGQSPQLLIYRMSNLASGVPDRFSGSGSGTAFTLRISRVEAEDVGVYYCMQHLEYPFTFGGGTKLEIKR

SEQ ID NO: 40 - protein, 118 aa, Artificial Sequence
  heavy chain variable region of antibody 8C7
QIQLVQSGPELKKPGETVKISCKASGYTFTNYGMNWVKQAPGKGLKWMGWINTNTGEPTYAEEFKGRFAFSLETSASTAYLQINNLKNEDTATYFCARSGGSSPFAYWGQGTLVTVSA

SEQ ID NO: 41 - protein, 15 aa, Artificial Sequence
  CDR L1 of antibody 1F5
RASKSVSTSGYSYMH

SEQ ID NO: 42 - protein, 7 aa, Artificial Sequence
  CDR L2 of antibody 1F5
RASNLES

SEQ ID NO: 43 - protein, 9 aa, Artificial Sequence
  CDR L3 of antibody 1F5
QHSRELPLT

SEQ ID NO: 44 - protein, 5 aa, Artificial Sequence
  CDR H1 of antibody 1F5
DYSVH

SEQ ID NO: 45 - protein, 7 aa, Artificial Sequence
  CDR H1 of antibody 1F5
GYTFTDY

SEQ ID NO: 46 - protein, 10 aa, Artificial Sequence
  CDR H1 of antibody 1F5
GYTFTDYSVH

SEQ ID NO: 47 - protein, 17 aa, Artificial Sequence
  CDR H2 of antibody 1F5
WINTETGEPTYADDFKG

SEQ ID NO: 48 - protein, 5 aa, Artificial Sequence
  CDR H2 of antibody 1F5
NTETG

SEQ ID NO: 49 - protein, 9 aa, Artificial Sequence
  CDR H2 of antibody 1F5
WINTETGEP

SEQ ID NO: 50 - protein, 8 aa, Artificial Sequence
  CDR H3 of antibody 1F5
AGGNAFAY

SEQ ID NO: 51 - protein, 16 aa, Artificial Sequence
  CDR L1 of antibody 8C7
RSSKSLLQSNGNTFLY

SEQ ID NO: 52 - protein, 7 aa, Artificial Sequence
  CDR L2 of antibody 8C7
RMSNLAS

SEQ ID NO: 53 - protein, 9 aa, Artificial Sequence
  CDR L3 of antibody 8C7
MQHLEYPFT

SEQ ID NO: 54 - protein, 5 aa, Artificial Sequence
  CDR H1 of antibody 8C7
NYGMN

SEQ ID NO: 55 - protein, 7 aa, Artificial Sequence
  CDR H1 of antibody 8C7
GYTFTNY

SEQ ID NO: 56 - protein, 10 aa, Artificial Sequence
  CDR H1 of antibody 8C7
GYTFTNYGMN

SEQ ID NO: 57 - protein, 17 aa, Artificial Sequence
  CDR H2 of antibody 8C7
WINTNTGEPTYAEEFKG

SEQ ID NO: 58 - protein, 5 aa, Artificial Sequence
  CDR H2 of antibody 8C7
NTNTG

SEQ ID NO: 59 - protein, 9 aa, Artificial Sequence
  CDR H2 of antibody 8C7
WINTNTGEP

SEQ ID NO: 60 - protein, 9 aa, Artificial Sequence
  CDR H3 of antibody 8C7
SGGSSPFAY

SEQ ID NO: 61 - protein, 6 aa, Homo sapiens
NGTYQT

(19) United States
(12) Patent Application Publication
(10) Pub. No.: US 2018/0154064 A1
(43) Pub. Date: Jun. 7, 2018

(54) METHODS AND DEVICES FOR REMOVAL OF IMMUNOSUPPRESSIVE LIGANDS

(71) Applicant: Novelogics Biotechnology, Inc., Vancouver (CA)

(72) Inventor: Ian Wayne CHENEY, Port Coquitlam (CA)

(21) Appl. No.: 15/881,304

(22) Filed: Jan. 26, 2018

Related U.S. Application Data
(62) Division of application No. 14/776,648, filed on Sep. 14, 2015, now abandoned.
(60) Provisional application No. 61/940,373, filed on Feb. 15, 2014.
(60) Provisional application No. 61/852,493, filed on Mar. 15, 2013.

(51) Int. Cl. A61M 1/36; C07K 16/28; A61K 35/16; C07K 17/10; A61K 35/14

(52) U.S. Cl. CPC A61M 1/3687; A61M 1/3603; C07K 16/2833; A61K 35/14; C07K 17/10; A61M 2202/07; A61M 1/3693; A61K 35/16

(57) ABSTRACT

The present disclosure relates to methods of removing soluble NKG2D ligands, including soluble MICA, soluble MICB and soluble ULBP proteins, from blood to treat diseases characterized by abnormal levels of soluble NKG2D ligands. Further provided are systems and devices for carrying out the therapeutic methods.

### CROSS REFERENCE TO RELATED APPLICATIONS

This application claims the benefit of U.S. Provisional Application No. 61/940,373, filed Feb. 15, 2014 and U.S. Provisional Application No. 61/852,493, filed Mar. 15, 2013. The contents of each cited priority application are incorporated herein by reference in their entirety.

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “NBI-002_ST25.txt”, a creation date of Mar. 15, 2014, and a size of 61 kilobytes. The sequence listing filed via EFS-Web is part of the specification and is hereby incorporated herein by reference in its entirety.

### TECHNICAL FIELD

The present invention relates generally to a method, system and device for treatment of diseases through selective removal of soluble immune modulators.

### BACKGROUND

Cancer rates worldwide are projected to continue rising as human life span increases and as mass lifestyle changes occur in the developing world. Thus, cancer remains a major cause of human morbidity and mortality even with the advent of modern and more efficacious targeted oncology medicines. There is a continuing need for novel therapeutic strategies, especially those that are capable of consistently providing or stimulating protective immunity as has been predicted for a number of immunotherapy treatments.

Oncology immunotherapy is designed to stimulate the body's immune system to fight tumors. Local immunotherapy administers a treatment into an affected area, thereby causing recruitment of immune cells, robust inflammation and consequently tumor shrinkage. Systemic immunotherapy treats the whole body by administering an agent, such as the protein interferon alpha, that is also capable of shrinking tumors. Immunotherapy can also be considered non-specific if it improves overall cancer-fighting abilities by stimulating the entire immune system, and it can be considered targeted if the treatment specifically directs the immune system to destroy cancer cells.

A key issue surrounding the interplay between the immune system, transformed cancerous cells, and use of immunotherapy is how tumor cells are able to avoid detection and survive in the face of an apparently normal and intact immune defense system bent on destroying them. A significant amount of research concerning the innate immunosurveillance system and its interaction with stressed and transformed cells has suggested that this particular arm of the immune system is suppressed in situations where moderate to advanced cancers are present. This is accomplished to a large extent by the release of decoy molecules from the tumor cells that neutralize the immunosurveillance system both locally and systemically (see, e.g., Ashiru et al., 2010, Cancer Res. 70:481-9). In fact, many viruses have evolved similar mechanisms for interfering with immune defense systems and thus avoid immune detection during their infection cycles (see, e.g., Jonjic et al., 2008, Curr Opin Immunol. 20(1): 30-8). Thus, where attenuation of immunosurveillance by decoy molecules is involved in the disease process, methods of countering the decoy molecules can provide therapeutic approaches for treating the disease.

### SUMMARY

The present disclosure provides methods, systems, and devices for removing soluble forms of NKG2D (sNKG2D) ligands from a subject where such removal is desirable. As further discussed herein, sNKG2D ligands, such as soluble MIC and soluble ULBP proteins, are elevated in some diseases, such as cancers and viral infections. These soluble forms shed from disease cells can act as decoy molecules and interact with their cognate receptors, particularly the NKG2D receptor involved in activating natural killer (NK) cells and CD8⁺ T cells. The cell-bound forms of the NKG2D ligands are induced during cellular stress, such as during viral infection and in cancers, and renders the cell susceptible to NK mediated cell killing However, the shed sNKG2D ligands can downregulate the NKG2D receptor, resulting in immunosuppression and attenuation of immunesurveillance by effector cells. Hence, the removal of these shed forms may enhance a subject's immune response, and form therapeutic strategies for treating the diseases, either independently or in combination with certain therapeutic agents.

Enhancing immune response as contemplated herein also includes one or more of the following: upregulation of T cell (e.g., γδ T cell, αβ T cell), natural killer (NK) cell, natural killer T (NKT) cell, and B cell function. In some embodiments, upregulation of one or more of T cell (e.g., γδ T cell, αβ T cell), natural killer (NK) cell, natural killer T (NKT) cell, and B cell function includes enhancement and/or endowment of activity capable of inhibiting cancer progression.

In some embodiments, inhibiting cancer progression as contemplated herein can be accomplished mainly by cytolysis of tumor cells, e.g., by direct induction of tumor cell apoptosis, induction of tumor cell cytolysis through stimulation of intrinsic host antitumor responses, induction of tumor cell apoptosis through stimulation of intrinsic host antitumor responses, inhibition of tumor cell metastasis, inhibition of tumor cell proliferation, and induction of senescence in the tumor cell.

Accordingly, in one aspect, the present disclosure provides a method of treating a subject suffering from a disease characterized by elevated levels of a soluble NKG2D (sNKG2D) ligand, the method comprising:
obtaining blood from a subject afflicted with a disease characterized by elevated levels of the sNKG2D ligand;
contacting or treating the blood or plasma fraction of the blood with a binding agent that binds specifically to the sNKG2D ligand under suitable conditions for complex formation between the binding moiety and sNKG2D ligand;
separating or removing the blood or plasma fraction from complexes of binding moiety and sNKG2D ligand; and
returning or reinfusing the blood or plasma fraction to the subject.

In some embodiments, the sNKG2D ligand comprises a soluble MICA (sMICA) or soluble MICB (sMICB) protein. In some embodiments, the sNKG2D ligand comprises a soluble ULBP protein (sULBP), such as soluble ULBP1 (sULBP1), soluble ULBP2 (sULBP2), soluble ULBP3 (sULBP3), soluble ULBP4 (sULBP4), soluble ULBP5 (sULBP5), or soluble ULPB6 (sULBP6).

In some embodiments, the binding agent comprises an antibody that binds specifically to the sNKG2D ligand. In some embodiments, the antibody binds specifically to sMICA and/or sMICA. In some embodiments, the antibody binds specifically to the alpha-1 domain, alpha-2 domain, and/or alpha-3 domain of MICA and/or MICB.

In some embodiments, the binding agent comprises an antibody that binds specifically to a sULBP protein. In some embodiments, the antibody binds specifically to sULBP1, sULBP2, sULBP3, sULBP4, sULBP5 or sULBP6. In some embodiments, the antibody binds specifically to the alpha-1 domain and/or the alpha-2 domain of a ULBP protein, for example, ULBP2. and/or ULBP3.

In some embodiments, the binding agent that binds specifically to a sNKG2D ligand comprises a receptor for a NKG2D ligand. Such receptors include, among others, the NKG2D receptor, including species homologs of the human NKG2D receptor; human cytomegalovirus (HCMV) UL16 viral protein; HCMV UL142 viral protein; human herpes virus-7 (HHV-7) U21 viral protein, or functional variants or fragments thereof.

Generally, the binding agents are immobilized on a solid carrier to effect efficient removal of sNGK2D ligand and treatment of the subject's blood or plasma fraction. In some embodiments, the solid carrier comprises water insoluble carriers, particularly water insoluble porous carriers. The solid carriers can be in various forms, including, by way of example and not limitation, particles, tubes, membranes, or channels. Exemplary solid carriers include, among others, agarose, dextran, polyacrylamide, silica, polysulfone, cellulose, polyamide, polyether, polyethylene, polypropylene, polyester, polyvinyl, and derivatives and mixtures thereof.

In another aspect, the present disclosure also provides a system for carrying out the therapeutic methods herein. Accordingly, in some embodiments, the system comprises
a plasma separator capable of separating plasma fraction from blood cell fraction;
a chamber containing a binding agent capable of specifically binding a sNKG2D ligand, wherein the binding agent is immobilized on a solid carrier; and
a pump for moving the separated plasma fraction through the chamber.

In some embodiments, the chamber of the system can comprise a column containing the solid carrier. In some embodiments, the system comprises two or more chambers, which can be used in series or in parallel, either simultaneously, alternately, or sequentially. In some embodiments, the system comprises two or more pumps, such as a first pump for transporting the blood through the plasma separator and a second pump for transporting the plasma fraction from the chamber and reinfusion of reconstituted blood into the subject. Numerous variations of the system are contemplated in light of the descriptions in the present disclosure.

In some embodiments, the system comprises an apheresis system. Accordingly, in another aspect, the present disclosure provides an apheresis device comprising a solid carrier capable of being contacted with blood or plasma, wherein the solid carrier comprises a binding agent that binds specifically to a sNKG2D ligand. In some embodiments, the solid carrier is contained in a chamber, such as a column, as described in the detailed description.

The methods, systems and devices of the present disclosure can be used to treat various diseases and disorders characterized by abnormal levels of a sNKG2D ligand, including diseases characterized by elevated levels of sMIC (sMIC⁺) or sULBP (sULBP⁺) ligands. Such diseases or disorders include, among others, sMIC⁺ and/or sULBP⁺ tumors, hematologic malignancies, and viral infections. In some embodiments, the therapeutic treatments can be used alone, or in combination with other therapeutic agents used to treat the relevant disorder.

In a further aspect, provided are kits for use in the methods, systems and devices of the disclosure. In some embodiments, the kit comprises a solid carrier with immobilized binding agent that binds specifically to a sMIC and/or sULBP protein. In some embodiments, the kit can comprise a chamber, such as a column, wherein the chamber comprises a solid carrier with immobilized binding agent that binds specifically to a sMIC and/or sULBP protein.

### BRIEF DESCRIPTION OF THE FIGURES

FIG. 1A and FIG. 1B depict an exemplary amino acid sequence corresponding to a complete human MICA polypeptide (Allele *001: NCBI accession no. NP_000238.1) (SEQ ID NO:1), and an exemplary amino acid sequence corresponding to a complete human MICB polypeptide (Allele *001: UniProtKB accession no. Q29980.1) (SEQ ID NO:2), respectively.

FIG. 1C and FIG. 1D depict the amino acid sequence of the extracellular alpha-3 domain of MICA protein of the MICA*001 allele, amino acid residues 205-297 (SEQ ID NO:3); and an amino acid sequence of the extracellular alpha-3 domain of MICB protein of the MICB*001 allele, amino acid residues 205-297 (SEQ ID NO:4), respectively. Amino acid numbering is based on the unprocessed MICA and MICB proteins. Amino acid numbering based on the processed, mature MICA*001 and MICB*001 corresponds to amino acid residues 182 to 274 for MICA and amino acid residues 182 to 274 for MICB.

FIG. 2 depicts an exemplary nucleotide sequence corresponding to the human MICA cDNA (Allele *001: NCBI accession no. NM_000247.2) (SEQ ID NO:5). The coding region is underlined.

FIG. 3 depicts an exemplary nucleotide sequence corresponding to human MICB cDNA (Allele *001: GenBank accession no. X91625.1) (SEQ ID NO:6). The coding region is underlined.

FIG. 4 depicts an exemplary amino acid sequence corresponding to a complete human NKG2D amino acid sequence (NCBI accession no. NP_031386.2) (SEQ ID NO:7).

FIG. 5 depicts an exemplary nucleotide sequence encoding human NKG2D receptor (NCBI accession no. NM_007360.3) (SEQ ID NO:8). The coding region is underlined.

FIG. 6 depicts the amino acid sequences of NKG2D receptors from various mammals. A—Mouse (mus musculus) killer cell lectin-like receptor subfamily K, member 1, transcript variant 2 (NCBI accession no. NP_001076791.1) (SEQ ID NO:9). B—Rat (rattus norvegicus) NKG2D type II integral membrane protein (NCBI accession no. NP_598196.1) (SEQ ID NO:10). C—Rhesus monkey (macaca mulatta) NKG2D protein (NCBI accession no. NP_001028061) (SEQ ID NO:11). D—Cynomolgus monkey (macaca fascicularis) NKG2D receptor (GenBank accession no. EHH66071.1) (SEQ ID NO:12). E—Marmoset (callithrix jacchus) NKG2D receptor isoform 1 (NCBI accession no. NP_001244178) (SEQ ID NO:13). F—Chimpanzee (pan troglodytes) NKG2D receptor (Genbank No. accession no. AAF86971.1) (SEQ ID NO:14).

FIG. 7 depicts exemplary amino acid sequences of human ULBP proteins. A—Human ULBP1 NKG2D ligand 1 precursor (NCBI accession no. NP_079494.1) (SEQ ID NO:15). B—Human ULBP2 NKG2D ligand 2 precursor (NCBI accession no. NP_079493.1) (SEQ ID NO:16). C—Human NKG2D ligand 3 precursor (NCBI accession no. NP_078794.1) (SEQ ID NO:17). D—Human ULBP4 NKG2D ligand 4 isoform 2 precursor (NCBI accession no. NP_001230254.1) (SEQ ID NO:18). E—Human ULBP5 retinoic acid early transcript 1G protein precursor (NCBI accession no. NP_001001788.2) (SEQ ID NO:19). F—Human ULBP6 retinoic acid early transcript 1L protein precursor (NCBI accession no. NP_570970.2) (SEQ ID NO:20).

FIG. 8 depicts an exemplary amino acid sequence of HCMV UL16 viral protein (GenBank: accession no. ABV71546.1: Strain AD169) (SEQ ID NO:21).

FIG. 9 depicts an exemplary amino acid sequence of HCMV UL142 viral protein (Genbank accession no. AAR31516.1) (SEQ ID NO:22).

FIG. 10 depicts an exemplary amino acid sequence of HHV-7 U21 viral protein (NCBI accession no. YP_073761.1: Strain J1) (SEQ ID NO:23).

FIG. 11 depicts the amino acid sequences of the variable regions of antibody 1F5 and 8C7, with the CDRs delineated using Kabat scheme highlighted. A—Amino acid sequence of light chain variable region of antibody 1F5 (SEQ ID NO:37). B—Amino acid sequence of heavy chain variable region of antibody 1F5 (SEQ ID NO:38). C—Amino acid sequence of light chain variable region of antibody 8C7 (SEQ ID NO:39). D—Amino acid sequence of light chain variable region of antibody 8C7 (SEQ ID NO:40).

FIG. 12 depicts the amino acid sequence of CDRs in the variable region of antibody 1F5 and 8C7. A—CDR L1 (SEQ ID NO:41), CDR L2 (SEQ ID NO:42), CDR L3 (SEQ ID NO:43), CDR H1 (SEQ ID NO:44; SEQ ID NO:45; SEQ ID NO:46), CDR H2 (SEQ ID NO:47; SEQ ID NO:48; SEQ ID NO:49) and CDR H3 (SEQ ID NO:50), delineated by methods of Kabat, Chothia, and AbM for antibody 1F5. B—CDR L1 (SEQ ID NO:51), CDR L2 (SEQ ID NO:52), CDR L3 (SEQ ID NO:53), CDR H1 (SEQ ID NO:54; SEQ ID NO:55; SEQ ID NO:56), CDR H2 (SEQ ID NO:57; SEQ ID NO:58; SEQ ID NO:59)and CDR H3 (SEQ ID NO:60), delineated by methods of Kabat, Chothia, and AbM for antibody 8C7. Where the CDRs assessments result in sequence differences, the method used to delineate the specific CDR sequence is indicated.

### DETAILED DESCRIPTION

The present disclosure provides methods for treating diseases or disorders characterized by elevated levels of soluble forms of NKG2D ligands, e.g., MICA, MICB, ULBP1, ULBP2, ULBP3, ULBP4, ULBP5, and ULBP6, by removing the soluble NKG2D ligand from a subject to limit the immunosuppressive effects of the circulating soluble forms, and thereby enhance the subject's own immune response against the disease or disorder. Removal of the soluble NKG2D can also be used to increase the efficacy of drug treatments used to treat the disease. Further provided in the disclosure are systems and devices to carry out the treatment methods.

Before various embodiments of the present invention are further described, it is to be understood that this disclosure is not limited to particular embodiments described, and as such may, of course, vary. It is also to be understood that the terminology used herein is for the purposes of describing particular embodiments only, and is not intended to be limiting.

It is also to be noted that as used herein and in the appended claims, the singular forms “a,” “an” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In addition, the use of “or” means “and/or” unless stated otherwise Similarly, “comprise,” “comprises,” “comprising,” “include,” “includes,” and “including” are interchangeable and not intended to be limiting. Where descriptions of various embodiments use the term “comprising,” those skilled in the art would understand that in some specific instances, an embodiment can be alternatively described using language “consisting essentially of” or “consisting of.”

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Although any methods and materials similar or equivalent to those described herein can also be used in the practice or testing of the present invention, the preferred methods and materials are now described. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In some embodiments, methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Accordingly, the following terms are intended to have the following meanings:

As used herein, the term “binding moiety” and “binding agent” are used interchangeably herein to refer to any molecule or part thereof that can bind specifically to another molecule.

As used herein, the term “specific binding agent to a soluble NKG2D ligand” refers to a specific binding agent that binds specifically to any portion of a soluble NKG2D ligand, such as MICA, MICB, ULBP1, ULBP2, ULBP3, ULBP4, ULBP5, or ULBP6. In some embodiments, a specific binding agent to a soluble NKG2D ligand is an antibody or a functional fragment thereof.

As used herein, the term “functional” refers to a form of a molecule which possesses either the native biological activity of the naturally existing molecule of its type, or any specific desired activity, for example as judged by its ability to bind to ligand molecules. Examples of “functional” polypeptides include an antibody binding specifically to an antigen through its antigen-binding site, and a NKG2D receptor molecule capable of binding to its ligand.

As used herein, the term “binds specifically to” refers to the ability of a binding agent to bind to a target molecule with greater affinity than it binds to a non-target. In some embodiments, specific binding refers to binding for a target with an affinity that is at least 10, 50, 100, 250, 500, 1000, times or greater than the affinity for a non-target. As used herein, “binds specifically” in the context of any antibody refers to an antibody that binds specifically to an antigen or epitope, such as with a high affinity, and does not significantly bind other unrelated antigens or epitopes.

As used herein, the term “antibody” is used in the broadest sense and refers to an immunoglobulin or fragment thereof, and encompasses any such polypeptide comprising an antigen-binding fragment of an antibody. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively Immunoglobulin classes may also be further classified into subclasses, including IgG subclasses IgG₁, IgG₂, IgG₃, and IgG₄; and IgA subclasses IgA₁ and IgA₂. The term includes but is not limited to polyclonal, monoclonal, monospecific, multispecific (e.g., bispecific antibodies), natural, humanized, human, single-chain, chimeric, synthetic, recombinant, hybrid, mutated, grafted, antibody fragments (e.g., a portion of a full-length antibody, generally the antigen binding or variable region thereof, e.g., Fab, Fab′, F(ab′)2, and Fv fragments) and in vitro generated antibodies so long as they exhibit the desired biological activity. The term also includes single chain antibodies, e.g., single chain Fv (sFv or scFv) antibodies, in which a variable heavy and a variable light chain are joined together, directly or through a peptide linker, to form a continuous polypeptide.

As used herein, the term “isolated” refers to a change from a natural state, that is, changed and/or removed from its original environment. For example, a polynucleotide or polypeptide (e.g., antibody) naturally present in an organism is not “isolated,” but the same polynucleotide or polypeptide when separated from a natural co-existing substance by the action of a human is “isolated.” Thus, an “isolated” antibody” is one which has been separated and/or recovered from a component of its natural environment.

As used herein, the term “purified antibody” refers to an antibody preparation in which the antibody is at least 80% or greater, at least 85% or greater, at least 90% or greater, at least 95% or greater by weight as compared to other contaminants (e.g., other proteins) in the preparation, such as by determination using SDS-PAGE under reducing or non-reducing conditions.

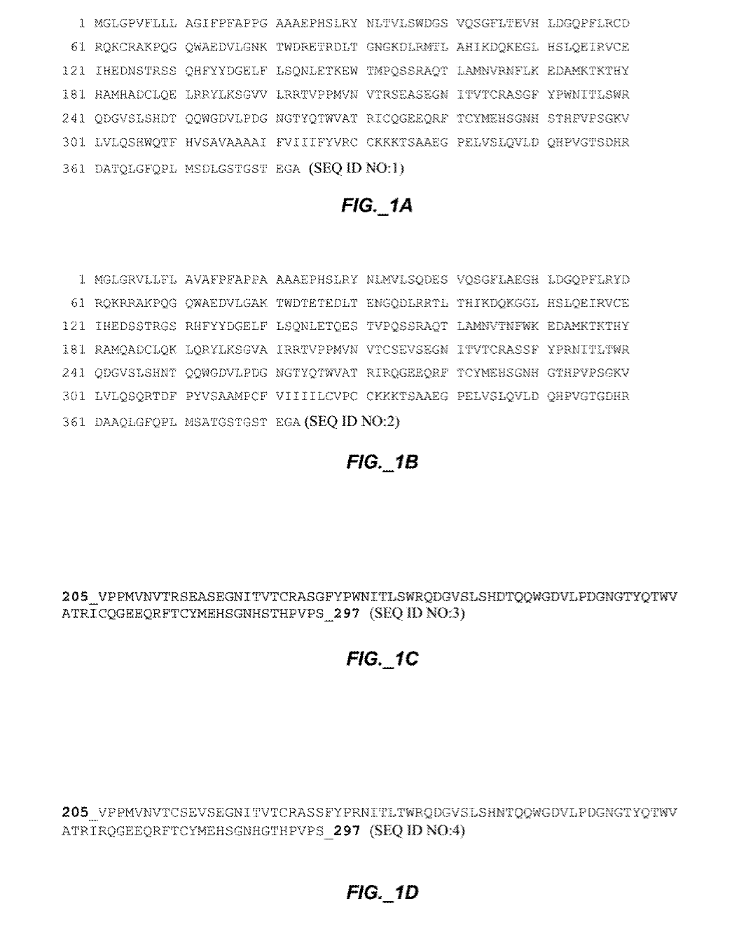

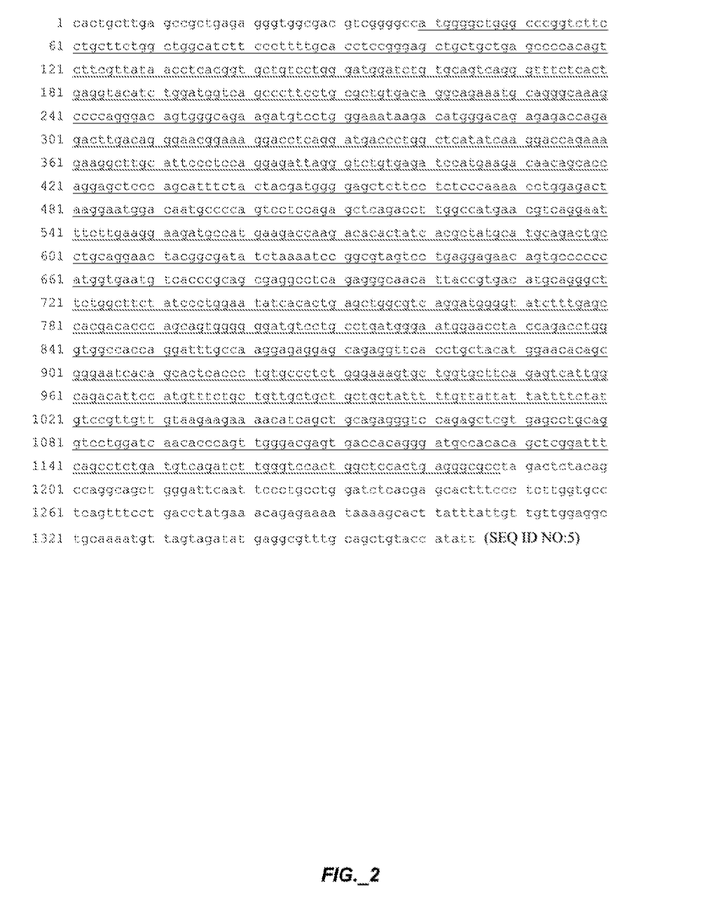

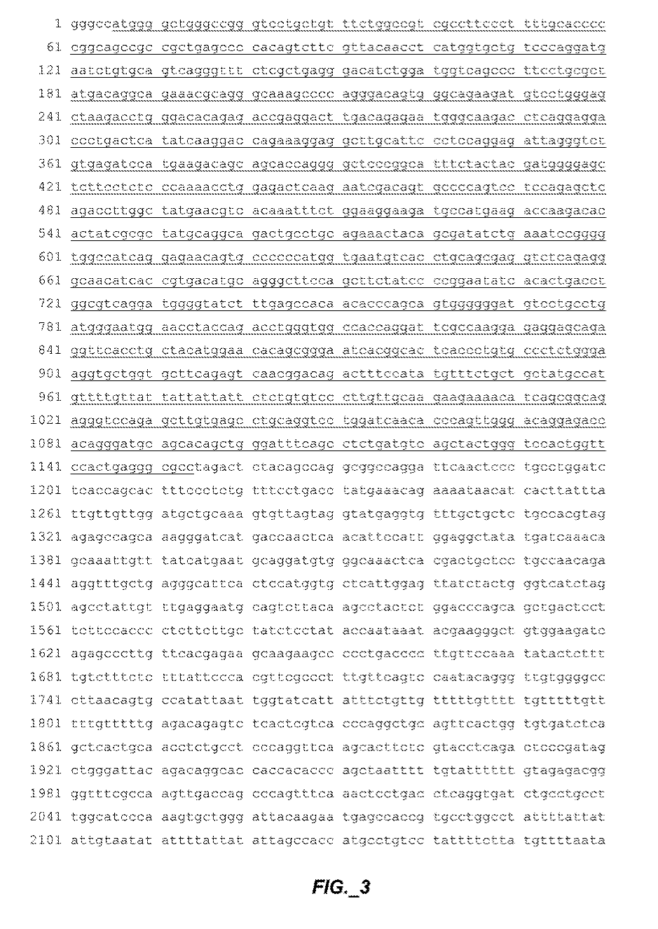

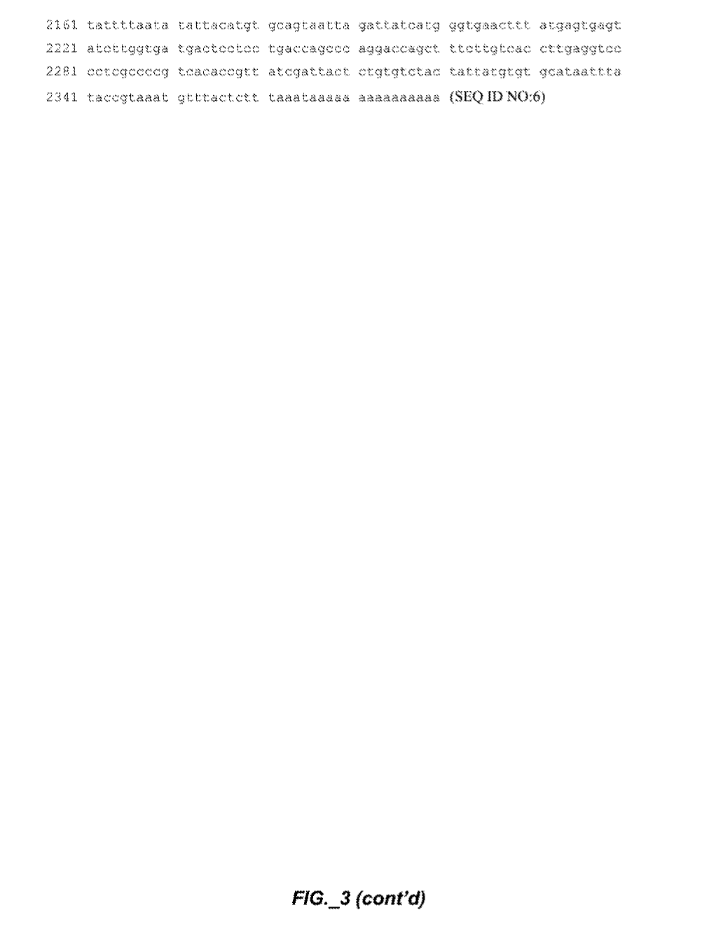

As used herein, the term “extracellular domain” and “ectodomain” are used interchangeably when used in reference to a membrane bound protein and refers to the portion of the protein that is exposed on the extracellular side of a lipid bilayer of a cell. For example, the extracellular domain of MICA is from amino acid residue at about 24 to about 299 of an unprocessed full length MICA protein, where the amino acid numbering is based on the MICA protein of the MICA*001 allele. In some embodiments, the extracellular domain of MICB is from amino acid residue at about 24 to about 299 of an unprocessed full length MICB protein, where the amino acid numbering is based on the MICB protein of the MICB*001 allele. It is to be understood that the polypeptide region defining the extracellular domain of MICA and MICB are approximate and, in some embodiments, may extend to about amino acid residue 307. An exemplary unprocessed full length MICA protein is presented in FIG. 1A (SEQ ID NO:1), and an exemplary unprocessed full length MICB protein is presented in FIG. 1B (SEQ ID NO:2).

As used herein, the term “membrane bound form” in the context of a protein or polypeptide refers to the protein or polypeptide containing the extracellular domain or portions thereof attached to at least a membrane anchoring domain, for example transmembrane domain or a GPI anchoring domain. A membrane bound form may or may not include the intracellular domain.

As used herein, the term “NKG2D ligand” refers to a binding partner that binds specifically to an NKG2D receptor. Exemplary ligands include MICA, MICB, ULBP1, ULBP2, ULBP3, ULBP4, ULBPS, ULBP6, and functional fragments thereof, such as soluble forms of MIC and ULBP ligands.

As used herein, the term “soluble NKG2D ligand” or “sNKG2D ligand” refers to a NKG2D ligand which is not attached or tethered to a cell and thus exists extracellularly. Generally, soluble NKG2D ligand lacks the domain that attaches the ligand to the cell, such as the transmembrane or GPI anchoring domain. In some embodiments, the sNKG2D ligand is functional in binding to the NKG2D receptor.

As used herein, the term “shedding” or “shed” in reference to a NKG2D ligand refers to release of a soluble extracellular domain fragment of a NKG2D ligand from the cell surface of a cell that expresses the NKG2D ligand. Such shedding may be caused by proteolytic cleavage of cell surface NKG2D ligand resulting in release of an extracellular domain fragment from the cell surface. In some embodiments, the soluble extracellular domain or fragment thereof may be encoded by an alternate transcript.

As used herein, the term “receptor” in the context of a NKG2D ligand refers to a molecule that binds specifically to a NKG2D ligand. Exemplary receptors for NKG2D ligand include the NKG2D receptor, human cytomegalovirus (HCMV) UL16 viral protein, human cytomegalovirus (HCMV) UL142 viral protein, and human herpes virus -7 (HHV-7) U21 viral protein.

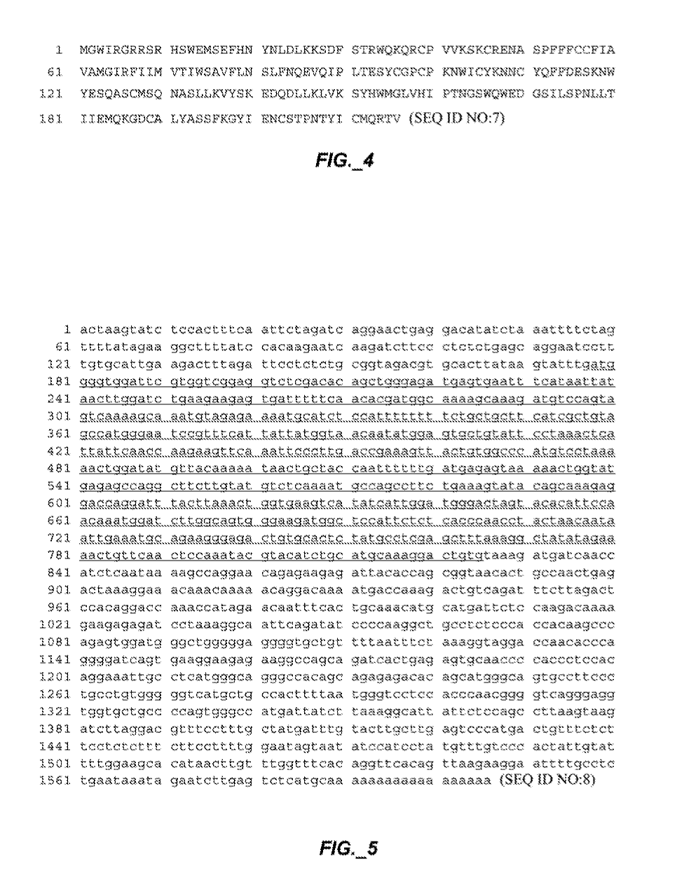

As used herein, the term “Natural Killer Group 2D”, “NKG2D” and “NKG2D receptor” refer to an activating cell surface molecule that is found on numerous types of immune cells, particularly NK cells, CD8⁺ T cells, some CD4⁺ T cells, and γδ T cells. NKG2D is also referred to as killer cell lectin-like receptor, subfamily C, member 4, or as KLRC4. The terms NKG2D and NKG2D receptor includes variants, isoforms, and species homologs of human NKG2D receptor (see, e.g., the isoforms described in Diefenbach et al., 2002, Nat Immunol. 3(12):1142-9). NKG2D is a type II transmembrane protein with an extracellular C-type (i.e., Ca²⁺-binding) lectin-like domain but lacking the Ca²⁺ binding site. It can form heterodimers with adapter proteins such as DAP10 or DAP12, and recognizes protein ligands that include MICA, MICB, ULBP1, ULBP2, ULBP3, ULBP4, ULBP5, and ULBP6. It is to be understood that any activity attributed herein to NKG2D, e.g., cell activation, recognition by antibodies, etc., can also refer to NKG2D-including complexes such as NKG2D-DAP10 or NKG2D-DAP12 heterodimers. Interaction of a NKG2D-bearing immune effector cell, for example an NK cell, with stressed or diseased cells expressing a NKG2D ligand, such as MICA or MICB, enhances the cellular immune response against the stressed/diseased cell. An amino acid sequence of an exemplary human NKG2D receptor is presented in FIG. 4 (SEQ ID NO:7). The nucleotide sequence encoding the human NKG2D receptor is presented in FIG. 5 (SEQ ID NO:8).

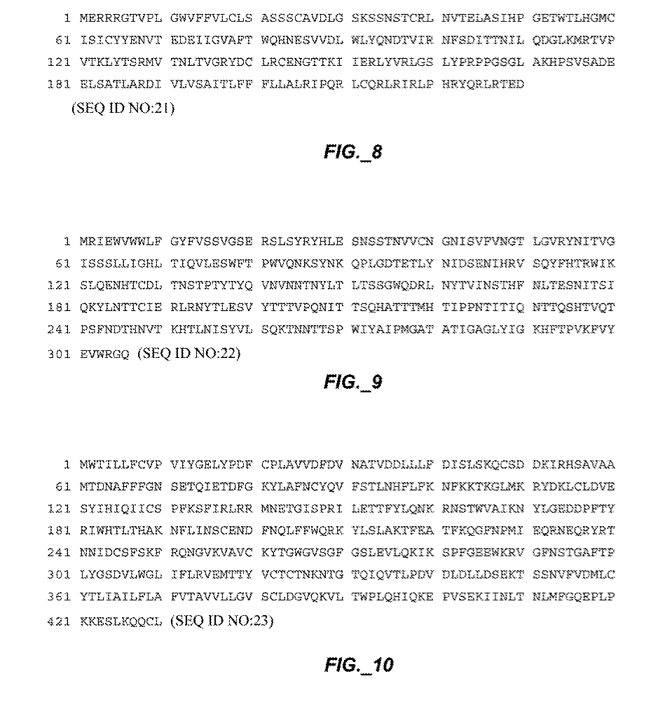

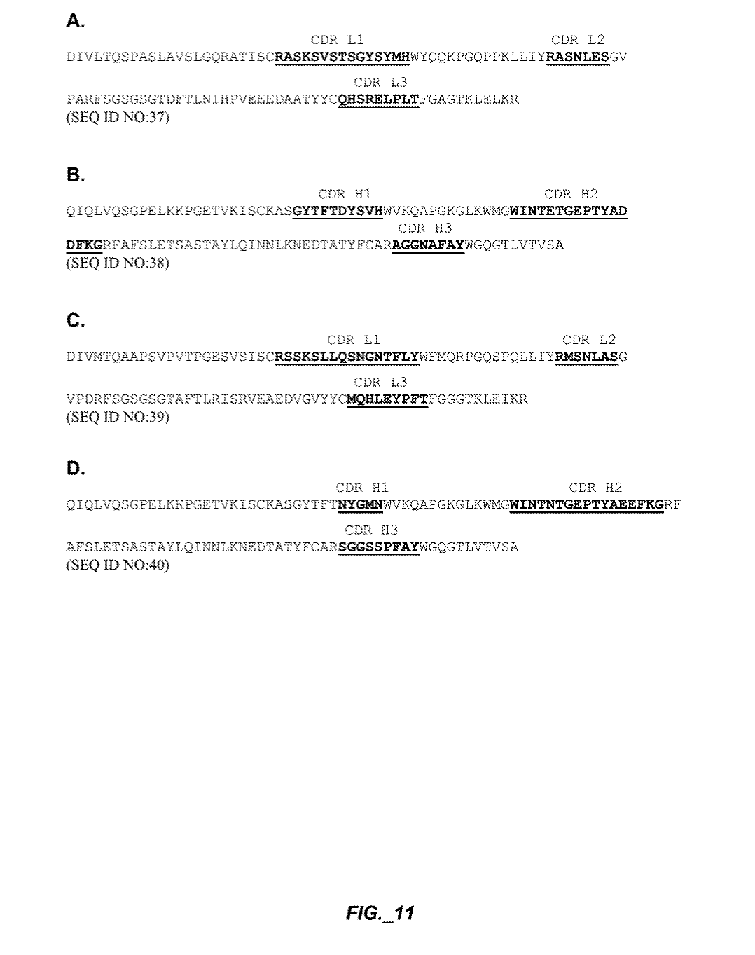

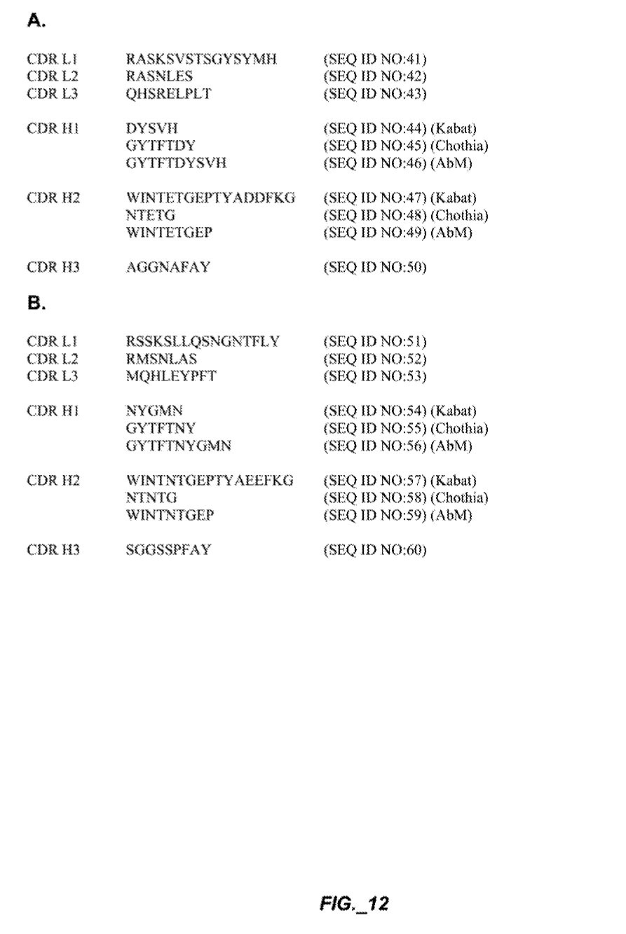

As used herein, the term “human cytomegalovirus UL16” or “HCMV UL16” viral protein refers to a membrane glycoprotein encoded in the UL regions of the human cytomegalovirus genome. UL16 protein can inhibit MHC class I antigen presentation by binding to particular NKG2D ligands. An exemplary amino acid sequence of HCMV UL16 is presented in FIG. 8 (SEQ ID NO:21).

As used herein, the term “human cytomegalovirus UL142” or “HCMV UL142” viral protein refers to a MHC class I-related glycoprotein encoded by certain strains of HCMV and is characterized by its ability to downregulate expression of certain NKG2D ligands, particularly MICA. The gene is predicted to encode a protein having alpha-1 and alpha-2 domains similar to the structure of other MHC class I proteins but contains a truncated alpha-3 domain (see, e.g., Wills et al., 2005, J Immunol. 175(11):7457-65). An exemplary amino acid sequence of HCMV UL142 is presented in FIG. 9 (SEQ ID NO:22).

As used herein, the term “human herpes virus -7 U21” or “HHV-7 U21” viral protein refers to a type 1 membrane protein encoded by human herpes virus-7 (HHV-7), with a predicted transmembrane domain, a cleavable signal sequence, and a short cytoplasmic tail (see, e.g., Hudson et al., 2001, J Virol. 75(24):12347-58). The protein associates with certain NKG2D ligands, particularly MICA, MICB and ULBP1, reducing cell surface expression of the ligands (see, e.g., Schneider and Hudson, 2011, PLoS Pathogens 7(11):e1002362). An exemplary amino acid sequence of HHV-7 U21 is presented in FIG. 10 (SEQ ID NO:23).

As used herein, the term “MICA” refers to MHC class I chain-related gene A protein (MICA), including variants, isoforms, and species homologs of human MICA, and includes fragments of MICA having functional MICA activity. Unlike HLA class I protein, MICA does not associate with β2 microglobulin. MICA expression is stress induced, and the protein acts as a ligand for natural killer cell (NK) receptor NKG2D. MICA protein comprises three extracellular Ig-like domains, i.e., alpha-1, alpha-2 and alpha-3, a transmembrane domain, and an intracellular domain. The protein is expressed at low levels in cells of the gastric epithelium, endothelial cells and fibroblasts and in the cytoplasm of keratinocytes and monocytes. An exemplary sequence of MICA is available as NCBI Accession Nos. NP_000238.1, and is presented in FIG. 1A (SEQ ID NO:1) of the present disclosure. Other exemplary MICA sequences can be found in U.S. patent publication 20110311561, incorporated herein by reference.

As used herein, the term “MICB” refers to MHC class I chain-related gene B protein (MICB), including variants, isoforms, and species homologs of human MICB, and includes fragments of MICB having functional MICB activity. Unlike HLA class I, the MICB protein does not associate with β2 microglobulin. MICB expression is stress induced and the protein acts as a ligand for natural killer cell (NK) receptor NKG2D. MICB has about 84% sequence identity to MICA. MICB protein comprises three extracellular Ig-like domains, i.e., alpha-1, alpha-2 and alpha-3, a transmembrane domain, and an intracellular domain. The protein is expressed at low levels in the gastric epithelium, endothelial cells and fibroblasts and in the cytoplasm of keratinocytes and monocytes. An exemplary sequence of MICB is available as UniProtKB accession number Q29980.1, and is presented in FIG. 1B (SEQ ID NO:2) of the present disclosure. Other exemplary MICB sequences can be found in U.S. patent publication 20110311561, incorporated herein by reference.

As used herein, the term “soluble MICA” or “sMICA” refers to a MICA protein containing the alpha-1, alpha-2, and alpha-3 domains but which is not attached or tethered to a cell and thus exists extracellularly. Generally, soluble MICA lacks the transmembrane domain. In some embodiments, the sMICA is functional in binding to the NKG2D receptor. As used herein, sMICA encompasses forms released from cells by proteolysis, which forms can be variable because of non-specificity of the proteolytic process. Exemplary sMICA comprises a polypeptide containing amino acid residues from about 24 to about 297 of the unprocessed full length MICA presented in FIG. 1A (SEQ ID NO:1).

As used herein, the term “soluble MICB” or “sMICB” refers to a MICB protein containing the alpha-1, alpha-2, and alpha-3 domains of the MICB protein but which is not attached or tethered to a cell and thus exists extracellularly. Generally, soluble MICB lacks the transmembrane domain. As used herein, sMICB encompasses forms released from cells by proteolysis, which forms can be variable because of non-specificity of the proteolytic process. Exemplary sMICB comprises a polypeptide of amino acid residues from about 24 to about 297 of the unprocessed full length MICB presented in FIG. 1B (SEQ ID NO:2).

As used herein, the term “full length MIC” refers to a MIC protein containing the alpha-1, alpha-2, and alpha-3 domains, the transmembrane domain, and the intracellular domain. “Unprocessed full length MIC protein” refers to a MIC protein that has not been processed following translation while a “full length mature MIC protein” or “full length processed MIC protein” refers to the processed form of the MIC protein, for example a MIC protein having a leader peptide removed. The full length unprocessed and the full length mature, processed proteins can vary in length due to the existence of polymorphisms. In some embodiments, the total unprocessed length (containing a leader sequence) can range from about 332 to about 388 amino acids for MICA and, in some embodiments, is about 383 amino acids for MICB. A processed MIC protein (with leader sequences removed) can range from about 309 to about 365 amino acids for MICA and about 360 amino acids for MICB. Exemplary unprocessed full length MIC proteins are set forth in FIG. 1A (SEQ ID NO:1) for MICA and FIG. 1B (SEQ ID NO:2) for MICB. Other exemplary full length MICA and MICB sequences can be found in U.S. patent publication 20110311561 and International patent publication WO2013117647, incorporated herein by reference.

As used herein, the term “alpha-1 domain” of a MIC protein (e.g., MICA and MICB) refers to amino terminal proximal Ig-like region (i.e., G-like domain) on the extracellular domain of MICA and MICB proteins (see, e.g., Frigoul and Lefranc, 2005, Recent Res Devel Human Genet. 3:95-145; incorporated herein by reference). An exemplary alpha-1 domain of MICA contains amino acid residues from about 24 to about 108 of unprocessed MICA protein of the MICA*001 allele. An exemplary alpha-1 domain of MICB contains amino acid residues from about 24 to about 108 of unprocessed MICB protein of the MICB*001 allele.

As used herein, the term “alpha-2 domain” of a MIC protein (e.g., MICA and MICB) refers to the second Ig-like region (i.e., G-like domain) on the extracellular domain of MICA and MICB proteins (see, e.g., Frigoul, A. and Lefranc, 2005, Recent Res Devel Human Genet. 3:95-145, incorporated herein by reference). An exemplary alpha-2 domain of MICA contains amino acid residues from about 109 to about 201 of unprocessed MICA protein of the MICA*001 allele. An exemplary alpha-2 domain of MICB protein contains amino acid residues from about 109 to about 201 of unprocessed MICB protein of the MICB*001 allele.

As used herein, the term “alpha-3 domain” of a MIC protein (e.g., MICA and MICB) refers to the transmembrane proximal region, also referred to as the C-like region on the extracellular domain of MICA and MICB proteins (see, e.g., Frigoul and Lefranc, 2005, Recent Res Devel Human Genet. 3:95-145, incorporated herein by reference). In some embodiments, the alpha-3 domain contains the disulfide bond formed between two cysteine residues in the alpha-3 domain. An exemplary alpha-3 domain of MICA contains amino acid residues from about 205 to about 296 or from about 205 to about 297 of unprocessed MICA protein of the MICA*001 allele (FIG. 1C: SEQ ID NO:3). An exemplary alpha-3 domain of MICB protein contains amino acid residues from about 205 to about 296 or from about 205 to about 297 of unprocessed MICB protein of the MICB*001 allele (FIG. 1D: SEQ ID NO:4).

As used herein, the term “ULBP protein” refers to members of the MHC class I-related molecules having a characteristic organization for the unprocessed protein that includes a N-terminal signal sequence, centrally located alpha-1 and alpha-2 domains, and a C-terminal cell membrane association domain, which can be a glycosylphosphatidylinositol (GPI) anchoring domain or a transmembrane domain. Some species of ULBP protein have a cytoplasmic domain. Generally, ULBP proteins have weak amino acid sequence identity to MICA/MICB proteins. ULBP family members are ligands for the effector cell receptor NKG2D, and are known to activate NK cells. As used herein. “ULBP protein” includes active variants, isoforms, and species homologs of human ULBP protein, and includes fragments having NKG2D receptor binding activity. ULBP family members appear to elicit at least some of their effects on NK cells by activating JAK2, STATS, ERK MAP kinase, and Akt/PKB (see, e.g., Sutherland et al., 2001. Immunol Rev. 181:185-92).

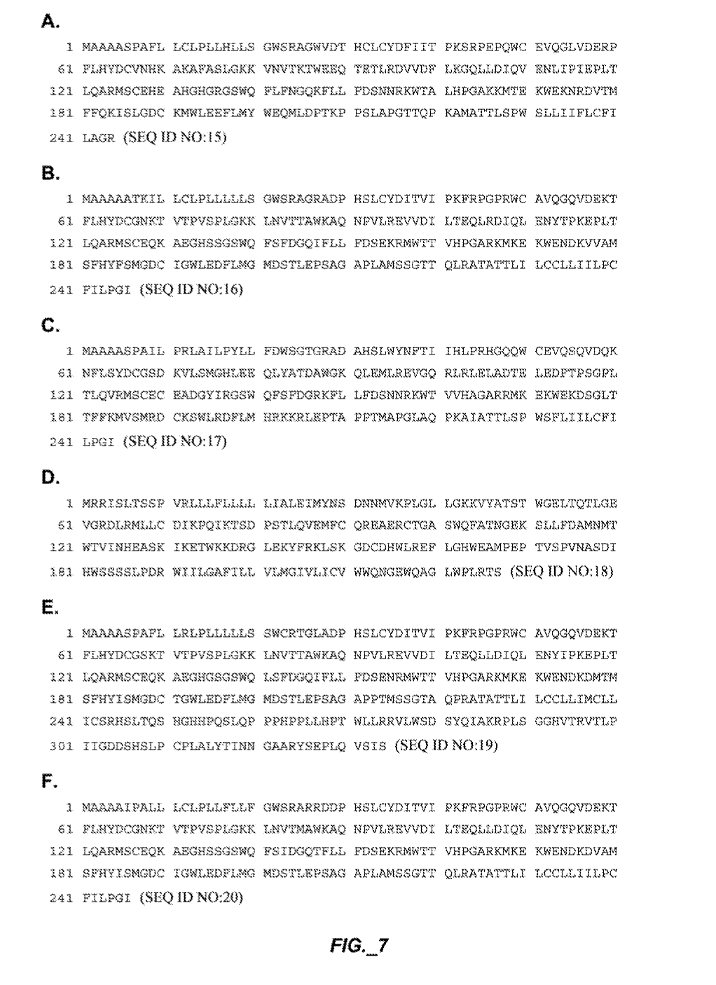

As used herein, the term “full length ULBP protein” refers to a ULBP protein containing the α1 and α2 domains, and when present, the transmembrane domain and the cytoplasmic domain, or a GPI anchoring domain. “Unprocessed full length ULBP protein” refers to a ULBP protein that has not been processed following translation. The full length mature, processed ULBP protein, which refers to the processed form, for example having the leader peptide removed, can vary due to the existence of polymorphisms and splicing variants. Exemplary unprocessed full length ULBP proteins are presented in FIG. 7, A to F: A—ULBP1 (SEQ ID NO:15); B—ULBP2 (SEQ ID NO:16); C—ULBP3 (SEQ ID NO:17); D—ULBP4 (SEQ ID NO:18); E—ULBPS (SEQ ID NO:19); and F—ULBP6 (SEQ ID NO:20).

As used herein, the term “alpha-1 domain” of an ULBP protein refers to the amino terminal proximal Ig-like region (i.e., G-like domain) on the extracellular domain of ULBP proteins. For example, the alpha-1 domain of ULBP1 contains amino acid residues from about 29 to about 117 of the unprocessed full length ULBP1 protein shown in FIG. 7, A (SEQ ID NO:15).

As used herein, the term “alpha-2 domain” of an ULBP protein refers to the cell membrane proximal Ig-like region (i.e., G-like domain) on the extracellular domain of ULBP proteins. Exemplary alpha-2 domain of ULBP1 contains amino acid residues from about 118 to about 208 of the full length ULBP1 protein shown in FIG. 7, A (SEQ ID NO:15).

As used herein, the term “ULBP1”, also described as “retinoic acid early transcript 1 protein” or “RAET1”, refers to a member of the MHC class I family, including variants, isoforms, and species homologs of human ULBP1. The protein functions as a ligand for receptor NKG2D. ULBP1 protein activates multiple signaling pathways in primary NK cells. The C terminal membrane association domain in ULBP1 comprises a GPI domain. ULBP1 is weakly homologous with MICA and MICB and has about 55% to 60% amino acid sequence identity to ULBP2 and ULBP3. Exemplary sequence of human ULBP1 is available as NCBI accession no. NP_079494.1 (FIG. 7, A: SEQ ID NO:15).

As used herein, the term “ULBP2”, also described as “retinoic acid early transcript 1H protein” or “RAET1H”, refers to a member of the MHC class I family, including variants, isoforms, and species homologs of human ULBP2. The protein functions acts as a ligand for receptor NKG2D. ULBP2 activates multiple signaling pathways in primary NK cells. The C terminal membrane association domain in ULBP2 comprises a GPI domain. ULBP2 is weakly homologous with MICA and MICB and has about 55% and 60% amino acid sequence identity to ULBP1 and ULBP3. Exemplary sequence of human ULBP2 is available as NCBI accession no. NP_079493.1 (FIG. 7, B: SEQ ID NO:16).

As used herein, the term “ULBP3”, also described as “retinoic acid early transcript 1N protein” or “RAET1N”, refers to a member of the MHC class I family, including variants, isoforms, and species homologs of human ULBP3. The protein functions as a ligand for receptor NKG2D. The C terminal membrane association domain in ULBP2 comprises a GPI anchoring domain. ULBP3 activates multiple signaling pathways in primary NK cells. ULBP3 is weakly homologous with MICA and MICB. Exemplary sequence of human ULBP3 is available as NCBI accession no. NP_078794.1 (FIG. 7, C: SEQ ID NO:17).

As used herein, the term “ULBP4”, also described as “retinoic acid early transcript 1E protein” or “RAET1E”, refers to a member of the MHC class I family, including variants, isoforms, and species homologs of human ULBP4. The protein functions as a ligand for receptor NKG2D. The C terminal region of ULBP4 comprises a transmembrane domain and a cytoplasmic domain, (see, e.g., U.S. patent publication US20090274699), in contrast to the GPI anchored domain in ULBP1, ULBP2 and ULBP3. ULBP4 is involved in activating NK cells through its binding to receptor NKG2D and induces NK-mediated lysis (see, e.g., Kong et al., 2009, Blood 114(2):310-17). ULBP4 has higher sequence identity to ULBP3 than ULBP1 and ULBP2. Exemplary amino acid sequences of human ULBP4 are available as NCBI accession nos. NP_001230254.1 (FIG. 7, D: SEQ ID NO:18); NP 001230256.1; NP 001230257.1; and NP 631904.1.

As used herein, the term “ULBP5”, also described as “retinoic acid early transcript 1G protein” or “RAET1G”, refers to a member of the MHC class I family, including variants, isoforms, and species homologs of human ULBP5. The C-terminal region of the protein has a transmembrane domain and a cytoplasmic domain, similar to ULBP4. ULBP5 is involved in activating NK cells and NK cell-mediated cytotoxicity through its binding to receptor NKG2D. ULBP5 is expressed frequently in cell lines derived from epithelial cancers, and in primary breast cancers. Exemplary sequence of human ULBP5 is available as NCBI accession no. NP_001001788.2 (FIG. 7, E: SEQ ID NO:19).

As used herein, the term “ULBP6”, also described as “retinoic acid early transcript 1L protein” or “RAET1L”, refers to a member of the MHC class I family, including variants, isoforms, and species homologs of human ULBP6. ULBP6 contains a GPI anchoring domain, similar to ULBP1, ULBP2, and ULBP3. ULBP6 is involved in activating NK cells and NK cell mediated cytotoxicity through its binding to receptor NKG2D. Exemplary sequence of human ULBP6 is available as NCBI accession no. NP_570970.2 (FIG. 7, F: SEQ ID NO:20).

As used herein, the term “soluble ULBP” or “sULBP” refers to ULBP proteins containing the alpha-1 and alpha-2 domains but which are not attached or tethered to a cell and thus exist extracellularly. Generally, sULBP lacks the GPI anchoring or the transmembrane domain. In some embodiments, the sULBP is functional in binding to NKG2D receptor.

As used herein, the term “soluble ULBP1” or “sULBP1” refers to a ULBP1 protein containing the alpha-1 and alpha-2 domains of the ULBP1 protein but which is not attached or tethered to a cell and thus exists extracellularly. Generally, in some embodiments, sULBP1 lacks the GPI anchoring domain. In some embodiments, the sULBP1 is functional in binding to NKG2D receptor.

As used herein, the term “soluble ULBP2” or “sULBP2” refers to a ULBP2 protein containing the alpha-1 and alpha-2 domains of the ULBP2 protein but which is not attached or tethered to a cell and thus exists extracellularly. Generally, in some embodiments, sULBP2 lacks the GPI anchoring domain. In some embodiments, the sULBP2 is functional in binding to NKG2D receptor.

As used herein, the term “soluble ULBP3” or “sULBP3” refers to a ULBP3 protein containing the alpha-1 and alpha-2 domains of the ULBP3 protein but which is not attached or tethered to a cell and thus exists extracellularly. Generally, in some embodiments, sULBP3 lacks the GPI anchoring domain. In some embodiments, the sULBP3 is functional in binding to NKG2D receptor.

As used herein, the term “soluble ULBP4” or “sULBP4” refers to a ULBP4 protein containing the alpha-1 and alpha-2 domains of the ULBP4 protein but which is not attached or tethered to a cell and thus exists extracellularly. Generally, in some embodiments, sULBP4 lacks the transmembrane domain. In some embodiments, the sULBP4 is functional in binding to NKG2D receptor.

As used herein, the term “soluble ULBP5” or “sULBP5” refers to a ULBP5 protein containing the alpha-1 and alpha-2 domains of the ULBP5 protein but which is not attached or tethered to a cell and exists extracellularly. Generally, in some embodiments, sULBP5 lacks the transmembrane domain. In some embodiments, the sULBP5 is functional in binding to NKG2D receptor.

As used herein, the term “soluble ULBP6” or “sULBP6” refers to a ULBP6 protein containing the alpha-1 and alpha-2 domains of the ULBP6 protein but which is not attached or tethered to a cell and thus exists extracellularly. Generally, in some embodiments, sULBP6 lacks the GPI anchoring domain. In some embodiments, the sULBP6 is functional in binding to NKG2D receptor.

As used herein, the term “epitope” or “antigenic determinant” refers to that portion of an antigen capable of being recognized and specifically bound by a particular binding agent, e.g., an antibody. When the antigen is a polypeptide, epitopes can be formed from contiguous amino acids and/or noncontiguous amino acids juxtaposed by tertiary folding of a protein. Linear epitope is an epitope formed from contiguous amino acids on the linear sequence of amino acids. A linear epitope is typically retained upon protein denaturing. Conformational or structural epitope is an epitope composed of amino acid residues that are not contiguous and thus comprised of separated parts of the linear sequence of amino acids that are brought into proximity to one another by folding of the molecule, such as through secondary, tertiary, and/or quaternary structures. A conformational or structural epitope is typically lost upon protein denaturation. In some embodiments, an epitope can comprise at least 3, and more usually, at least 5 or 8-10 amino acids in a unique spatial conformation. Thus, an epitope encompasses a defined epitope in which only portions of the defined epitope bind an antibody. There are many methods known in the art for mapping and characterizing the location of epitopes on proteins, including solving the crystal structure of an antibody-antigen complex, competition assays, gene fragment expression assays, mutation assays, and synthetic peptide-based assays, as described, for example, in “Using Antibodies, A Laboratory Manual,” Harlow and Lane, eds., Chapter 11, Cold Spring Harbor Laboratory Press, Cold Spring Harbor, New York (1999).

As used herein, the term “cryptic epitope” refers to an epitope that is not exposed for recognition by a binding agent within a native structure, but is capable of being recognized when there is a disruption of the native structure that exposes the cryptic epitope. In the context of a protein, a cryptic epitope refers to a protein sequence that is not exposed for recognition within a native protein, but is capable of being recognized by a binding agent when there is a disruption of the native protein structure or when the epitope is separate from the native protein. Sequences that are not exposed or are only partially exposed in the native structure are potential cryptic epitopes. If an epitope is not exposed or only partially exposed, then it is likely that it is buried within the interior of the molecule. Candidate cryptic epitopes also can be identified, for example, by examining the three-dimensional structure of a native protein. In some embodiments, structural disruptions capable of exposing cryptic epitopes include denaturation and proteolysis. Separation of the cryptic epitope from the native protein can occur by proteolysis, synthesis of a protein fragment containing the epitope, or release of an extracellular portion of the native protein from a membrane, such as a cell surface membrane.

As used herein, the term “polymorphic” or “polymorphism” refers to the occurrence of two or more forms of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long. A polymorphic protein refers to occurrence of two or more forms of the protein due to polymorphisms in the encoding gene sequence.

As used herein, the term “allele” refers to the specific gene sequence at a locus, which is the position occupied by a segment of a specific sequence of base pairs along a gene sequence of DNA.

As used herein, the term “amino acid position” and “amino acid residue” are used interchangeably to refer to the position of an amino acid in a polypeptide chain In some embodiments, the amino acid residue can be represented as “XN”, where X represents the amino acid and the N represents its position in the polypeptide chain. Where two or more variations, e.g., polymorphisms, occur at the same amino acid position, the variations can be represented with a “/” separating the polymorphisms. A substitution of one amino acid residue with another amino acid residue at a specified residue position can be represented by XNY, where X represents the original amino acid, N represents the position in the polypeptide chain, and Y represents the replacement or substitute amino acid. When the terms are used to describe a polypeptide or peptide portion in reference to a larger polypeptide or protein, the first number referenced describes the position where the polypeptide or peptide begins (i.e., amino end) and the second referenced number describes where the polypeptide or peptide ends (i.e., carboxy end). For example, a peptide from amino acid position 190 to 196 of a processed full length MICA refers to a peptide in which its amino end is at position 190 of a processed full length MICA protein and the carboxy end is at position 196 of the processed full length MICA protein.

As used herein, the term “polyclonal” antibody refers to a composition comprising different antibody molecules which are capable of binding to or reacting with several different specific antigenic determinants on the same or on different antigens. A polyclonal antibody can also be considered to be a “cocktail of monoclonal antibodies”. The polyclonal antibodies may be of any origin, e.g., chimeric, humanized, or fully human.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Each monoclonal antibody is directed against a single determinant on the antigen. Monoclonal antibodies are highly specific. For example, the monoclonal antibodies to be used in accordance with the present disclosure may be made by the hybridoma method first described by Kohler et al., 1975, Nature 256:495-7, or may be made by recombinant DNA methods. The monoclonal antibodies may also be isolated, e.g., from phage antibody libraries.

As used herein, the term “chimeric antibody” refers to an antibody made up of components from at least two different sources. A chimeric antibody can comprise a portion of an antibody derived from a first species fused to another molecule, e.g., a portion of an antibody derived from a second species. In some embodiments, a chimeric antibody comprises a portion of an antibody derived from a non-human animal, e.g., mouse or rat, fused to a portion of an antibody derived from a human. In some embodiments, a chimeric antibody comprises all or a portion of a variable region of an antibody derived from a non-human animal fused to a constant region of an antibody derived from a human.

As used herein, the term “humanized antibody” refers to an antibody that comprises a donor antibody binding specificity, e.g., the complementarity determining region (CDR) of a donor antibody, such as a mouse monoclonal antibody, grafted onto human framework sequences. A “humanized antibody” as used herein typically binds to the same epitope as the donor antibody.

As used herein, the term “fully human antibody” or “human antibody” refers to an antibody that comprises human immunoglobulin protein sequences only. A fully human antibody may contain murine carbohydrate chains if produced in a mouse, in a mouse cell, or in a hybridoma derived from a mouse cell.

As used herein, the term “antibody fragment” or “antigen-binding moiety” refers to a portion of a full length antibody, generally the antigen binding or variable domain thereof. Examples of antibody fragments include Fab, Fab′, F(ab)2, and Fv fragments; diabodies; linear antibodies; single-chain antibody molecules; and multispecific antibodies formed from antibody fragments that bind two or more different antigens. Several examples of antibody fragments containing increased binding stoichiometries or variable valencies (2, 3 or 4) include triabodies, trivalent antibodies and trimerbodies, tetrabodies, tandabs, di-diabodies and (sc(Fv)2)2 molecules, and all can be used as molecular traps to bind with high affinity and avidity to soluble antigens (see, e.g., Cuesta et al., 2010, Trends Biotech., 28: 355-62).

As used herein, the term “single-chainFv” or “sFv” antibody fragments comprise the VH and VL domains of an antibody, where these domains are present in a single polypeptide chain. Generally, the Fv polypeptide further comprises a polypeptide linker between the VH and VL domains which enables the sFv to form the desired structure for antigen binding. For a review of sFv, see Pluckthun, in The Pharmacology of Monoclonal Antibodies, Vol. 113, Rosenburg and Moore, eds., pp. 269-315, Springer-Verlag, New York (1994).

As used herein, the term “multibodies” refers to multivalent constructs with several antigen-binding sites derived from antibodies, e.g., diabodies, bis-scFV, triabodies, tetrabodies. Bis-scFv has an overall structure close to diabodies, except that it is composed of only one polypeptide comprising four variable domains.

As used herein, the term “diabodies” refers to small antibody fragments with two antigen-binding sites, which comprise a heavy chain variable domain (VH) connected to a light chain variable domain (VL) in the same polypeptide chain (VH-VL). By using a linker that is short to allow pairing between the two domains on the same chain, the domains are forced to pair with the complementary domains of another chain and create two antigen-binding sites.

As used herein, the term “antigen binding domain” or “antigen binding portion” refers to the region or part of the antigen binding molecule that specifically binds to and complementary to part or all of an antigen. In some embodiments, an antigen binding domain may only bind to a particular part of the antigen (e.g., an epitope), particularly where the antigen is large. An antigen binding domain may comprise one or more antibody variable regions, particularly an antibody light chain variable region (VL) and an antibody heavy chain variable region (VH), and particularly the complementarity determining regions (CDRs) on each of the VH and VL chains.

As used herein, the term “variable region” and “variable domain” are used interchangeably to refer to the polypeptide region that differ extensively in sequence between antibodies and confers the binding and specificity characteristics of each particular antibody. The variable region in the heavy chain of an antibody is referred to as “VH” while the variable region in the light chain of an antibody is referred to as “VL”. The major variability in sequence is generally localized in three regions of the variable domain, denoted as “hypervariable regions” in each of the VL region and VH region, and forms the antigen binding site. The more conserved portions of the variable domains are referred to as the framework region.

As used herein, the term “complementarity-determining region” or “CDR” are used interchangeably to refer to non-contiguous antigen binding regions found within the variable region of the heavy and light chain polypeptides. In some embodiments, the CDRs are also described as “hypervariable regions”. Generally, naturally occurring antibodies comprise six CDRs, three in the VH (CDR H1 or H1; CDR H2 or H2; and CDR H3 or H3) and three in the VL (CDR L1 or L1; CDR L2 or L2; and CDR L3 or L3). The CDR domains have been delineated using various approaches, and it is to be understood that CDRs defined by the different approaches are to be encompassed herein. The “Kabat” approach for defining CDRs uses sequence variability and is the most commonly used (Kabat et al., 1991, “Sequences of Proteins of Immunological Interest, 5^{th} Ed.” NIH 1:688-96). “Chothia” uses the location of structural loops (Chothia and Lesk, 1987, J Mol Biol. 196:901-17). CDRs defined by “AbM” is a compromise between the Kabat and Chothia, and is delineated using Oxford Molecular AbM antibody modeling software (see, Martin et al., 1989, Proc. Natl Acad Sci USA. 86:9268; see also, world wide web site www.bioinf-org.uk/abs). The “Contact” CDR delineations are based on analysis of known antibody-antigen crystal structures (see, e.g., MacCallum et al., 1996, J. Mol. Biol. 262, 732-45). The CDRs delineated by these methods typically include overlapping or subsets of amino acid residues when compared to each other. Generally, the residues defining the CDRs using each of the approaches are noted in the following:

| CDR | KABAT | CHOTHIA | ABM | CONTACT |
|---|---|---|---|---|
| CDR L1 | 24-34 | 24-34 | 24-34 | 30-36 |
| CDR L2 | 50-56 | 50-56 | 50-56 | 46-55 |
| CDR L3 | 89-97 | 89-97 | 89-97 | 89-96 |
| CDR H1 | 31-35B | 26-32B | 26-35 | 30-35B |

(Kabat Numbering)

| | | | | |
|---|---|---|---|---|
| CDR H1 | 31-35 | 26-35 | 26-32 | 30-35 |

(Chothia Numbering)

| | | | | |
|---|---|---|---|---|
| CDR H2 | 50-65 | 52-56 | 50-58 | 47-58 |
| CDR H3 | 95-102 | 95-102 | 95-102 | 93-101 |

It is to be understood that the exact residue numbers which encompass a particular CDR will vary depending on the sequence and size of the CDR, and those skilled in the art can routinely determine which residues comprise a particular CDR given the amino acid sequence of the variable region of an antibody.

Kabat, supra, also defined a numbering system for variable domain sequences that is applicable to any antibody. One of skill in the art can assign this system of “Kabat numbering” to any variable domain sequence. Accordingly, unless otherwise specified, references to the number of specific amino acid residues in an antibody or antigen binding fragment are according to the Kabat numbering system. In some embodiments, the sequences relevant to variable regions and CDRs (e.g., SEQ ID NOS: 37-40 and SEQ ID NOS:41-60) are not numbered according to Kabat numbering system, but one of ordinary skill in the art will recognize that such sequences can be converted to the Kabat numbering system.

As used herein, the term “framework region” or “FR region” refers to amino acid residues that are part of the variable region but are not part of the CDRs (e.g., using the Kabat, Chothia, or AbM definition). The variable region of an antibody generally contains four FR regions: FR1, FR2, FR3 and FR4. Accordingly, the FR regions in a VL region appear in the following sequence: FR L1-CDR L1-FR_{L}2-CDR L2-FR_{L}3-CDR L3-FR_{L}4, while the FR regions in a VH region appear in the following sequence: FR1_{H}-CDR H1-FR_{H}2-CDR H2-FR_{H}3-CDR H3-FR_{H}4.

As used herein, the term “human consensus framework” refers to a framework that represents the most commonly occurring amino acid residues in a selection of human immunoglobulin VL or VH framework sequences. Generally, the selection of human immunoglobulin VL or VH sequences is from a subgroup of variable domain sequences. In some embodiments, the subgroups sequences is a subgroup presented in Kabat et al., supra. In some embodiments, for the VL the subgroup is subgroup kappa described in Kabat et al., supra. In some embodiments, for the VH the subgroup is subgroup III described in Kabat et al., supra.

As used herein, the term “constant region” or “constant domain” refers to a region of an immunoglobulin light chain or heavy chain that is distinct from the variable region. The constant domain of the heavy chain generally comprises at least one of: a CH1 domain, a Hinge (e.g., upper, middle, and/or lower hinge region), a CH2 domain, and a CH3 domain. For example, an antibody described herein may comprise a polypeptide comprising a CH1 domain; a polypeptide comprising a CH1 domain, at least a portion of a Hinge domain, and a CH2 domain; a polypeptide comprising a CH1 domain and a CH3 domain; a polypeptide comprising a CH1 domain, at least a portion of a Hinge domain, and a CH3 domain, or a polypeptide comprising a CH1 domain, at least a portion of a Hinge domain, a CH2 domain, and a CH3 domain. In some embodiments, a polypeptide comprises a polypeptide chain comprising a CH3 domain. The constant domain of a light chain is referred to a CL, and in some embodiments, can be a kappa or lambda constant region, However, it will be understood by one of ordinary skill in the art that these constant domains (e.g., the heavy chain or light chain) may be modified such that they vary in amino acid sequence from the naturally occurring immunoglobulin molecule.

As used herein, the term “Fc region” or “Fc portion” refers to the C terminal region of an immunoglobulin heavy chain The Fc region can be a native-sequence Fc region or a non-naturally occurring variant Fc region. Generally, the Fc region of an immunoglobulin comprises constant domains CH2 and CH3. Although the boundaries of the Fc region can vary, in some embodiments, the human IgG heavy chain Fc region can be defined to extend from an amino acid residue at position C226 or from P230 to the carboxy terminus thereof. In some embodiments, the “CH2 domain” of a human IgG Fc region, also denoted as “Cγ2”, usually extends from about amino acid residue 231 to about amino acid residue 340. In some embodiments, N-linked carbohydrate chains are interposed between the two CH2 domains of an intact native IgG molecule. In some embodiments, the CH3 domain” of a human IgG Fc region comprises residues C-terminal to the CH2 domain, e.g., from about amino acid residue 341 to about amino acid residue 447 of the Fc region. A “functional Fc region” possesses an “effector function” of a native sequence Fc region. Exemplary Fc “effector functions” include, among others, Clq binding; complement dependent cytotoxicity (CDC); Fc receptor binding; antibody dependent cell-mediated cytotoxicity (ADCC); phagocytosis; down regulation of cell-surface receptors (e.g., LT receptor), etc. Such effector functions generally require the Fc region to be combined with a binding domain (e.g., an antibody variable domain) and can be assessed using various assays known in the art.

As used herein, the term “binding affinity” refers to strength of the sum total of noncovalent interactions between a ligand and its binding partner. In some embodiments, binding affinity is the intrinsic affinity reflecting a one-to-one interaction between the ligand and binding partner. The affinity is generally expressed in terms of equilibrium association (K_{A}) or dissociation constants (K_{D}), which are in turn reciprocal ratios of dissociation (k_{off}) and association rate constants (kₒₙ).

As used herein, the term “fusion protein” or “fusion polypeptide” refers to a protein having at least two heterologous polypeptides covalently linked, either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order.

As used herein, the term “functional fragment” in the context of a protein or polypeptide refers to a fragment of a larger polypeptide that retains a desired biological property of the larger polypeptide.

As used herein, the term “subsequence” refers to a sequence of a nucleic acid or polypeptide which comprises a part of a longer sequence of a nucleic acid or polypeptide, respectively.

As used herein, the term “subject” refers to a mammal, including, but not limited to humans, non-human primates, and non-primate mammals, such as dogs, cats, goats, horses, and cows which is to be the recipient of a particular treatment. In some embodiments, the terms “subject” and “patient” are used interchangeably herein in reference to a human subject.

As used herein, the term “elevated” refers to above-normal levels of a disease marker or indicator that has a statistically significant correlation with the occurrence of the disease. The levels can be compared to appropriate controls, e.g., healthy subjects without the disease, to determine the levels that signal presence of the disease.

As used herein, the term “abnormal” or “abnormality” refers to a level or condition which is statistically different from the level or condition observed in organisms not suffering from such a disease or disorder and may be characterized as either an excess amount, intensity or duration of signal or a deficient amount, intensity or duration of signal. The abnormality may be realized as an abnormality in cell function, viability or differentiation state. An abnormal interaction level may also be either greater, or less than the normal level, and may impair the normal performance or function of the organism.

As used herein, the terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers.

As used herein, the term “proliferative disorder” and “proliferative disease” refer to disorders associated with abnormal cell proliferation such as cancer.

As used herein, the terms “tumor” and “neoplasm” as used herein refer to any mass of tissue that result from excessive cell growth or proliferation, either benign (noncancerous) or malignant (cancerous) including pre-cancerous lesions.

As used herein, the term “MIC⁺ disease or disorder” refers to a disease or disorder displaying elevated levels of MICA and/or MICB proteins or portions thereof, such as sMICA and sMICB, that are correlated with the occurrence of the disease or disorder.

As used herein, the term “MICA⁺ disease or disorder” refers to a disease or disorder displaying elevated levels of MICA protein or portions thereof, such as sMICA, that is correlated with the occurrence of the disease or disorder.

As used herein, the term “MICB⁺ disease or disorder” refers to a disease or disorder displaying elevated levels of MICB protein or portions thereof, such as sMICB, that is correlated with the occurrence of the disease or disorder.

As used herein, the term “MIC⁺ tumor” refers to a tumor or neoplasm characterized by elevated levels of a MIC protein or portions thereof, such as sMICA and sMICB.

As used herein, the term “MIC⁺ hematologic malignancy” refers to proliferative disorders of cells of the lymphoid or myeloid system characterized by elevated levels of a MIC protein or portions thereof, such as sMICA and sMICB. Lymphoid disorders include acute lymphocytic leukemia and chronic lymphoproliferative disorders (e.g., lymphoma, myeloma, and chronic lymphoid leukemias). Lymphomas include Hodgkin's disease and non-Hodgkin's lymphoma, precursor T-cell leukemia/lymphoma, follicular lymphoma, diffuse large B-cell lymphoma, mantle cell lymphoma, MALT lymphoma, Burkitt's lymphoma, B-cell chronic lymphocytic leukemia/lymphoma, peripheral T-cell lymphoma—not-otherwise-specified, and mycosis fungoides. Chronic lymphoid leukemias include T cell chronic lymphoid leukemias and B cell chronic lymphoid leukemias. Myeloid disorders include chronic myeloid disorders and acute myeloid leukemia. Chronic myeloid disorders include chronic myeloproliferative disorders and myelodysplastic syndrome. Chronic myeloproliferative disorders include angiogenic myeloid metaplasia, essential thrombocythemia, chronic myelogenous leukemia, polycythemia vera, and atypical myeloproliferative disorders. Atypical myeloproliferative disorders include atypical CML, chronic neutrophilic leukemia, mast cell disease, and chronic eosinophilic leukemia.

As used herein, the term “MIC⁺ viral infection” refers to a viral infection characterized by elevated levels of a MIC protein or portions thereof, such as sMICA and sMICB.

As used herein, the term “ULBP⁺ disease or disorder” refers to a disease or disorder characterized by elevated levels of any one or more of the ULBP proteins or portions thereof, particularly in the form of sULBP, that is correlated with the occurrence of the disease or disorder.

As used herein, the term “ULBP⁺ tumor” refers to a tumor or neoplasm characterized by elevated levels of a ULBP protein or portions thereof, such as sULBP.

As used herein, the term “ULBP⁺ hematologic malignancy” refers to proliferative disorders of cells of the lymphoid or myeloid system characterized by elevated levels of a ULBP protein or portions thereof, such as sULBP.

As used herein, the term “ULBP⁺ viral infection” refers to a viral infection characterized by elevated levels of a ULBP protein or portions thereof, such as sULBP.

As used herein, the term “ULBP1⁺ disease or disorder” refers to a disease or disorder characterized by elevated levels of ULBP1 protein or portions thereof, such as sULBP1, that is correlated with the occurrence of the disease or disorder.

As used herein, the term “ULBP2⁺ disease or disorder” refers to a disease or disorder characterized by elevated levels of ULBP2 protein or portions thereof, such as sULBP2, that is correlated with the occurrence of the disease or disorder.

As used herein, the term “ULBP3⁺ disease or disorder” refers to a disease or disorder characterized by elevated levels of ULBP3 protein or portions thereof, such as sULBP3, that is correlated with the occurrence of the disease or disorder.

As used herein, the term “ULBP4⁺ disease or disorder” refers to a disease or disorder characterized by elevated levels of ULBP4 protein or portions thereof, such as sULBP4, that is correlated with the occurrence of the disease or disorder.

As used herein, the term “ULBP5⁺ disease or disorder” refers to a disease or disorder characterized by elevated levels of ULBP5 protein or portions thereof, such as sULBP5, that is correlated with the occurrence of the disease or disorder.

As used herein, the term “ULBP6⁺ disease or disorder” refers to a disease or disorder characterized by elevated levels of ULBP6 protein or portions thereof, such as sULBP6, that is correlated with the occurrence of the disease or disorder.

As used herein, the term “treatment” or “treating” refers to a process that is intended to produce a beneficial change in the condition of a mammal, e.g., a human, often referred to as a patient. A beneficial change can, for example, include one or more of restoration of function; reduction of symptoms; reduction of severity; limitation or retardation of progression of a disease, disorder, or condition or prevention; or limitation or retardation of deterioration of a patient's condition, disease or disorder. In the context of a disease or disorder, a “therapy”, “treatment”, or “treatable” is meant the therapy achieves a desired pharmacologic and/or physiologic effect on the disease or disorder. The effect may be prophylactic in terms of completely or partially preventing the disease/disorder or symptom thereof and/or may be therapeutic in terms of a partial or complete cure for the disease/disorder and/or adverse effect attributable to the disease/disorder. The term includes: (a) preventing the disease from occurring in a subject which may be predisposed to the disease but has not yet been diagnosed as having it; (b) inhibiting the disease, i.e., arresting its development; or (c) relieving the disease, i.e., causing remission or regression of the disease. The therapeutic agent may be administered before, during or after the onset of the disease or disorder. The treatment of an ongoing disease, where the treatment stabilizes or reduces the undesirable clinical symptoms of the patient, is of particular interest. Such treatment is desirably performed prior to complete loss of function in the affected tissues.

As used herein, the term “monotherapy” refers to a treatment regimen based on the delivery of one therapeutically effective compound, whether administered as a single dose or several doses over time.

As used herein, the term “combination therapy” refers to a therapeutic regimen that involves the provision of at least two distinct therapies to achieve an indicated therapeutic effect. For example, a combination therapy may involve two or more distinct therapeutic treatments, for example, at least one of the treatment methods disclosed herein; and a chemotherapeutic or biologic agent. Alternatively, a combination therapy may involve at least one of the methods disclosed herein and/or one or more therapeutic agents, alone or together with the delivery of another treatment, such as radiation therapy and/or surgery.

As used herein, the term “immune stimulating agent” or “immuno activating agent” refers to an agent, such as a compound or composition, which enhances an immune response, e.g., as compared to the immune response in the absence of the immune stimulating agent.

As used herein, the term “vaccine” refers to a compound or composition which can be administered to humans or to animals in order to induce an immune system response; this immune system response can result in production of antibodies or result in the activation of certain cells, in particular antigen-presenting cells and immune system effector cells, such as T lymphocytes and B lymphocytes. The vaccine composition can be a composition for prophylactic purposes and/or for therapeutic purposes. As such, a “cancer vaccine” refers to a compound or composition which elicits an immune response against a cancer. The immune response can be against a broad spectrum of cancers or against a specific cancer.

As used herein, the term “apheresis” in the context of therapy refers to a process of removing a specific component from the blood, plasma, serum, or a fraction thereof, of a subject. In some embodiments of apheresis, whole blood is removed from the patient and, as a first stage, separates the plasma from the blood ex-vivo, and in a second stage treats the separated plasma by various techniques. The treated plasma and blood are recombined ex-vivo and returned to the patient. In some embodiments, apheresis also includes treatment of blood without separation of the plasma fraction. Although apheresis is typically done extracorporeally, the term as used herein includes treatment of the blood in vivo, for example, using implantable devices.

As used herein, the term “solid carrier” refers to an insoluble material used for immobilizing a molecular entity, particularly a biological molecule such as an antibody. The solid carrier can be non-porous or porous. The solid carrier can be any appropriate geometric form, including uniform or irregular particles, tubes and channels. In some embodiments, the solid carrier comprises water insoluble carriers, particularly water insoluble porous carriers.

As described above, in one aspect, the present disclosure provides methods of treating a subject afflicted with a disease or disorder characterized by abnormal (e.g., elevated) levels of a soluble NKG2D ligand, e.g., soluble MICA (sMICA), soluble MICB (sMICB), and soluble ULBP (sULBP), such as soluble ULBP2 (sULBP2), and soluble ULBP3 (sULBP3). The MIC and the ULBP proteins are members of the MHC Class I-related chain (MIC) family of proteins (Leelayuwat et al., 1994, Immunogenetics 40: 339-51; Bahram et al., 1994, Proc Natl Acad Sci USA. 91:6259-63; Fodil et al., 1996, Immunogenetics 44:351-7; Groh et al., 1999, Proc Natl Acad Sci USA. 96:6879-84; Bauer et al., 1999, Science 285(5428):727-9). The ULBP proteins were initially identified as binding targets of the human cytomegalovirus (HCMV) glycoprotein, UL16. The MIC and ULBP proteins act as ligands that bind to C-type lectin-like activating receptor Natural Killer Group 2D (NKG2D) on immune effector cells, including NK, NKT and both αβ and γδ CD8⁺ T cells. Homology analyses indicate that MIC ligands are highly conserved in most mammals, with the exception of the rodent family, and are weakly related to MHC class I proteins. The highly related MICA and MICB glycoproteins are about 84% identical at the amino acid sequence level (Bahram 1994, Proc. Natl. Acad. Sci. USA 91: 6259-63; Bahram, 1996, Immunogenetics 44:80-1; Bahram and Spies., 1996, Immunogenetics 43:230-3). The MICA and MICB proteins are stress-induced and although similar to MHC class I molecules, they do not associate with β2-microglobulin or bind peptides.

The ULBP proteins are homologous to each other but generally have weak homology to the MICA and MICB proteins. Similar to MICA and MICB, ULBP proteins do not associate with β2 microglobulin. The ULBP proteins have an alpha-1 and alpha-2-domain, but lack the alpha-3 domain found on MIC proteins. In addition, some ULBP proteins, such as ULBP1, ULBP2, ULBP3 and ULBP6 are anchored to the cell membrane via a GPI anchor while other members, such as ULBP4 and ULBPS, have a transmembrane domain. As with MICA and MICB, a known function of ULBP proteins is binding to NKG2D receptor and activating NK cell activity.

A significant amount of basic research concerning the innate immunosurveillance system and its interaction with stressed and transformed cells has suggested that this particular arm of the immune system is suppressed in situations where moderate to advanced cancers are present Immune suppression is accomplished to a large extent by the release of decoy molecules from the tumor cells with the distinct objective of neutralizing the immunosurveillance system both locally and systemically. In fact, many viruses have evolved similar mechanisms for interfering with these defense systems and thus avoiding immune detection during their infection cycles. The current state of the art for this surveillance system comprises the NKG2D and members of the MIC family and the related UL-16 binding proteins described above, including ULBP1, ULBP2, ULBP3, ULBP4, ULBPS and ULBP6 (see, e.g., Radosavljevic et al., 2002, Genomics 79:114-23; Leelayuwat et al., 1994, Immunogenetics 40:339-51; Bahram, 1994, Proc Natl Acad Sci USA. 91:6259-63; Fodil et al., 1996, Immunogenetics 44:351-7; Groh et al., 1999, Proc Natl Acad Sci USA. 96:6879-84; and Bauer et al., 1999, Science 285(5428):727-9). The interaction of NKG2D-bearing immune effector cells with stressed or diseased cells expressing MIC and/or ULBP ligands can create a cellular immune response against the stressed/diseased cell that culminates in the death of the ligand expressing cells. Binding of the MIC and ULBP ligands to NKG2D bearing immune cells stimulates the activation of naive T cells and can even induce cytotoxicity in the absence of appropriate TCR ligation.

In humans, the NKG2D receptor appears to function as a co-stimulatory molecule in association with DAP10 to impart the ligand binding signal to the interior of the cell via the phosphatidylinositol kinase (PI3K) pathway. The expression of NKG2D ligands has been reported in many types of tumors and is thought to be the result of gene expression arising from stimulation of heat shock promoter elements as well as the intracellular detection of DNA damage resulting from either environmental insult or the increasing level of genomic instability due to cancer. Some evidence (see, e.g., Groh et al., 2002, Nature 419:734-8) suggests that tumor-derived soluble MICA and MICB ligands that are shed from the surface of the tumor cells function like decoy molecules and lead to down-modulation of their cognate activating receptor NKG2D on immune effector cells such as NK, NKT and various CD8⁺ T cells. In so doing, this can result in an unusual situation whereby the soldiers of the innate defense system whose very job it is to seek and destroy transformed cells are shut down by the immunosuppressive actions of these decoy MIC ligand molecules. Through this mechanism, tumor cells are capable of hiding from the immune system and can continue to grow unabated. As a further consideration, persistent NKG2D ligand expression and shedding promote proliferation of normally rare, immunosuppressive NKG2D⁺ CD4⁺ T cells in cancer patients, and is directly correlated with serum concentration of sMICA thereby enabling NKG2D costimulation of T cell proliferation (Groh et al., 2006, Nat Immunol. 7:755-62).

In support of the adverse effect of these ligands, advanced cancer patients have been shown to have significantly elevated levels of soluble MIC immune decoy molecules (sMICA and/or sMICB) in their blood compared with healthy individuals (see, e.g., Groh et al., 2002, Nature 419:734-8; Salih et al., 2002, J Immunol. 169:4098-102; Salih et al., 2003, Blood 102:1389-96), and these high levels appear to directly correlate with both the clinical staging of the cancer and to poor clinical outcomes (Doubrovina et al., 2003, J Immunol. 171:6891-9; Wu et al., 2004, J Clin Invest. 114:560-8; Holdenreider et al., 2006, Intl J Cancer 118:684-7). Exemplary diseases that display elevated levels of sMICA and/or sMICB include, among others, gastric cancer, colon cancer, rectal cancer, lung cancers, breast cancers, cervical cancers, gliomas, chronic myelogenous leukemia, acute lymphocytic leukemia, Non-Hodgkin's Lymphoma (Salih et al., 2002, J Immunol. 169:409-12; Salih et al., 2003, Blood 102:1389-96; Boissel et al., 2006, J Immunol. 176:5108-16; Groh et al., 2002, Nature 419:734-8; Arreygue-Garcia et al., 2008, BMC Cancer 8:16; Eisele et al., 2006, Brain 129(9):2416-25); neuroblastoma (Raffaghello et al., 2004, Neoplasia 6:558-68); prostate cancer (Wu et al., 2004, J Clin Invest. 114:560-8); multiple myeloma (Rebmann et al., 2007, Clin Immunol. 123:114-20; Jinushi et al., 2008, Proc Natl Acad Sci USA. 105:1285-90); melanoma (Paschen et al., 2009, Clin Can Res. 15(16):5208-15); pancreatic ductal adenocarcinoma (Xu et al., 2009, BMC Cancer 11:194-204; Chang et al., 2011, PLOS One 6(5):e20029); Respiratory Syncytial Virus (RSV) infections (Zdrenghea et al., 2012, Eur Respir J. 39:712-20); HBV-induced Hepatocellular Carcinoma (Kumar et al., 2012, PLOS One 7(9):e44743); HCV-induced Hepatocellular Carcinoma (Lo et al., 2013, PLOS One 8(4):e61279) and HIV infection (Nolting et al., 2010, Virology 406:12-20; Matusali et al., 2013, FASEB J. 27:1-11).

Soluble forms of ULBP acting as potential immune decoy molecules have also been observed. Soluble forms of ULBP1, ULBP2 and ULBP4 have been shown to bind to NKG2D and downregulate NKG2D expression, resulting in suppression of NK cell activity (see, e.g., Song et al., 2006, Cell Immunol. 239(1):22-30). ULBP proteins are expressed in various human cancer cell lines as well as in cancer patients. By way of example and not limitation, ULBP1, ULBP2, and ULBP3 expression is found in Non-Hodgkin's Lymphoma (Salih et al., 2003, Blood 102:1389-96) and in gastric tumor cell lines (Song et al., 2006, Cellular Immunol. 239:22-30). Soluble ULBP2 is preferentially expressed in certain cervical cancers (Jimenez-Perez et al., 2012, BMC Immunol. 13:7) and glioma cell lines (Eisele et al., Brain 129(9):2416-25), and in patients with leukemia (Waldhauer and Steinle, 2006, Cancer Res. 66(5):2520-6; Onda et al., 2001, Biochem Biophys Res Commun 285(2):235-43) and pancreatic cancer (Chang et al., 2011, PLOS One 6(5): e20029). Elevated levels of soluble ULBP2 (sULBP2) are found in sera of melanoma and ovarian cancer patients and are correlated with poor prognosis (Paschen et al., 2009, Clin Cancer Res. 15(16):5208-15; Li et al., 2009, Cancer Immunol Immunother. 58(5):641-52). sULBP2 is also found in patients infected with HIV (Matusali et al., 2013, FASEB J. 27:1-11). ULBP5 is expressed in cell lines derived from epithelial cancers, and in primary breast cancer, and soluble forms of ULBP5 downregulate NKG2D receptor expression on NK cells Similarly, soluble ULBP4 and ULBP5 are expressed in various cancer cell lines and inhibit NKG2D-mediated NK cytotoxicity (Cao et al., 2007, J Biol Chem. 282(26):18922-8).

In light of the presence of soluble NKG2D ligands in various cancers and viral infections, and its impact on NKG2D receptor and immunesurveillance, the methods herein are directed to removal of the ligands from patients using binding agents that can bind specifically to the circulating soluble NKG2D ligands. Removal of the soluble NKG2D ligands can limit and/or reduce the immunosuppressive effects caused by the down modulation of its cognate receptor NKG2D, thereby enhancing the patient's own immune system in targeting the disease cells as well as increasing the efficacy of certain therapeutic agents, particularly those that act through enhancing the patient's own immune system, for example cancer vaccines and anti-CTLA4 antibodies.

Accordingly, in some embodiments, a method of treating a subject afflicted with a disease characterized by elevated levels of a soluble NKG2D ligand can comprise:
obtaining blood from a subject suffering from a disease characterized by elevated levels of a soluble NKG2D ligand;
contacting or treating the blood or plasma fraction of the blood with a binding agent that binds specifically to the soluble NKG2D ligand under suitable conditions for complex formation between the binding agent and soluble NKG2D ligand;
separating or removing the blood or plasma fraction from complexes of binding agent and soluble NKG2D ligand; and
returning or reinfusing the blood or plasma fraction to the subject.

In some embodiments, a method of treating a subject afflicted with a disease characterized by elevated levels of a soluble NKG2D ligand can comprise:
treating or contacting the subject's blood or plasma fraction of the blood extracorporeally with a binding agent that specifically binds a soluble NKG2D ligand;
separating or removing the blood or plasma fraction from complexes of binding agent and soluble NKG2D ligand; and
returning or reinfusing the blood or the plasma fraction of the blood to the subject.

In some embodiments, the soluble NKG2D ligand is capable of binding to receptor NKG2D. Without being bound by any postulated mechanism of action, the engagement of the NKG2D receptor by the soluble NKG2D ligand results in impairment of the immunosurveillance mechanism, particularly through attenuation of cell mediated killing resulting from downregulation of the receptor NKG2D.

In some embodiments, the plasma fraction is separated from the blood cell fraction, which includes red blood cells, white blood cells, and platelets. The separated plasma fraction is treated with the soluble NKG2D ligand binding agent under suitable conditions for complex formation between the binding agent and soluble NKG2D ligand. The treated plasma fraction is separated from the complexes of binding agent and soluble NKG2D ligand, and reconstituted with the blood cell fraction and returned to the patient. Generally, the binding agents are prepared aseptically so as not to contain endotoxin or other materials unacceptable for administration to a patient.

In some embodiments, the soluble NKG2D ligand comprises a soluble MICA (sMICA) and/or soluble MICB (sMICB) protein, and the binding agent binds specifically to sMICA and/or sMICB.

In some embodiments, the soluble NKG2D ligand comprises a soluble ULBP (sULBP) protein, and the binding agent binds specifically to the sULBP protein.

In some embodiments, the soluble NKG2D ligand comprises a soluble ULBP1 (sULBP1), and the binding agent binds specifically to sULBP1.

In some embodiments, the soluble NKG2D ligand comprises a soluble ULBP2 (sULBP2) protein, and the binding agent binds specifically to sULBP2.

In some embodiments, the soluble NKG2D ligand comprises a soluble ULBP3 (sULBP3) protein, and the binding agent binds specifically to sULBP3.

In some embodiments, the soluble NKG2D ligand comprises a soluble ULBP4 (sULBP4) protein, and the binding agent binds specifically to sULBP4.

In some embodiments, the soluble NKG2D ligand comprises a soluble ULBP5 (sULBP5) protein, and the binding agent binds specifically to sULBP5.

In some embodiments, the soluble NKG2D ligand comprises a soluble ULBP6 (sULBP6) protein, and the binding agent binds specifically to sULBP6.

In the embodiments herein, any binding agent that can bind specifically to the soluble NKG2D ligand can be used. In some embodiments, the binding agent, such as an antibody capable of binding a soluble NKG2D ligand, has an affinity (K_{A}=equilibrium association constant or the ratio of association rate constant kₒₙ/dissociation rate constant k_{off}) for the soluble NKG2D ligand in the range of about 10⁴ to about 10¹² M⁻¹, about 10⁵ to about 10¹² M⁻¹, about 10⁶ to about 10¹² M⁻¹, about 10⁷ to about 10¹² M⁻¹, about 10⁸ to about 10¹² M⁻¹, about 10⁷ to about 10¹¹M⁻¹, about 10⁸ to about 10¹¹M⁻¹, about 10⁷ to about 10¹⁰ M⁻¹, or about 10⁸ to about 10¹⁰ M⁻¹. In some embodiments, the binding agent has a K_{A} of at least about 1×10⁷ M⁻¹ or higher, at least about 1×10⁸ M⁻¹ or higher, at least about 1 x 10⁹ M⁻¹ or higher, at least about 1×10¹⁰ M⁻¹ or higher, at least about 1×10¹¹M⁻¹ or higher, or at least about 1×10¹² M⁻¹ or higher. In some embodiments, the binding agent comprises an antibody that binds specifically to MICA, MICB, ULBP1, ULBP2, ULBP3, ULBP4, ULBP5 or ULBP6, particularly MICA or MICB. In some embodiments, the antibody has a K_{A} of about 1×10⁹ M⁻¹ to about 1×10¹⁰ M⁻¹ or higher (e.g., affinity of antibody 1F5). In some embodiments, the antibody has a K_{A} or about 1×10⁸ M⁻¹ to about 1×10⁹ M⁻¹ or higher (e.g., affinity of antibody 8C7).

In some embodiments, the binding agent that can bind specifically to the soluble NKG2D ligand has an equilibrium dissociation constant (K_{D}=equilibrium dissociation constant or ratio of dissociation rate constant k_{off}/association rate constant kₒₙ) in the range of about 10⁻⁴ to about 10⁻¹² M, about 10⁻⁵ to about 10⁻¹² M, about 10⁻⁶ to about 10⁻¹² M, about 10⁻⁷ to about 10⁻¹² M, about 10⁻⁸ to about 10⁻¹² M, about 10⁻⁷ to about 10⁻¹¹M, about 10⁻⁸ to about 10⁻¹¹M, about 10⁻⁷ to about 10⁻¹⁰ M, or about 10⁻⁸ to about 10⁻¹⁰ M. In some embodiments, the binding agent has a K_{A} of about 1×10⁻⁷ M or less, about 1×10⁻⁸ Mor less, about 1×10⁻⁹ Mor less, about 1×10⁻¹⁰ Mor less, about 1×10⁻¹¹ Mor less, or about 1×10⁻¹² M or less. In some embodiments, the binding agent comprises an antibody that binds specifically to MICA, MICB, ULBP1, ULBP2, ULBP3, ULBP4, ULBP5 or ULBP6, particularly MICA or MICB. In some embodiments, the antibody has a K_{D} of the antibody 1F5 or antibody 8C7 described herein. In some embodiments, the antibody has a K_{D} of about 1×10⁻⁹ M to about 1×10⁻¹⁰ M or less (e.g., antibody 1F5). In some embodiments, the antibody has a K_{D} of about 5×10⁻⁹ M to about 1×10⁻¹⁰ M or less (e.g., antibody 8C7).

In some embodiments, the binding agent that can bind specifically to a soluble NKG2D ligand has a kₒₙ association rate constant in the range of about 10³ to about 10⁹ M⁻¹s¹ or greater, about 10⁴ to about 10⁹ M⁻'s⁻¹ or greater, about 10⁵ to about 10⁹ M⁻¹s⁻¹ or greater, about 10⁶ to about 10⁹ M⁻¹s⁻or greater, about 10⁷ to about 10⁹ M⁻'s⁻¹ or greater, about 10⁴ to about 10⁸ M⁻¹s⁻¹ or greater, or about 10⁵ to about 10⁸ M⁻'s⁻¹ or greater. In some embodiments, the binding agent has a kₒₙ association rate constant of at least about 1×10³ M⁻'s⁻¹ or greater, at least about 1×10⁴ M⁻¹s⁻¹ or greater, at least about 1×10⁵ M⁻¹s⁻¹ or greater, at least about 1×10⁶ M⁻¹s⁻¹ or greater, at least about 1×10⁷ M⁻¹s⁻¹ or greater, at least about 1×10⁸ M⁻¹s⁻¹ or greater, or at least about 1×10⁹ M⁻¹s⁻¹ or greater. In some embodiments, the binding agent comprises an antibody. In some embodiments, the binding agent comprises an antibody that binds specifically to MICA, MICB, ULBP1, ULBP2, ULBP3, ULBP4, ULBP5 or ULBP6, particularly MICA or MICB. In some embodiments, the antibody has a kₒₙ association rate constant for MICA characteristic of the antibody 1F5 or antibody 8C7 described herein.

In some embodiments, the binding agent that can bind specifically to a soluble NKG2D ligand has a k_{off} dissociation rate constant of about 10⁻³ to about 10⁻¹⁰ s⁻¹ or less, about 10⁻⁴ to about 10⁻¹⁰ s⁻¹ or less, about 10⁻⁵ to about 10⁻¹⁰ s⁻¹ or less, about 10⁻⁶ to about 10⁻¹⁰ s⁻¹ or less, about 10⁻⁷ to about 10⁻¹⁰ s⁻¹ or less, about 10⁻⁵ to about 10⁻⁹ s⁻¹ or less, about 10⁻⁶ to about 10⁻⁹ s⁻¹ or less, about 10⁻⁵ to about 10⁻⁸ s⁻¹ or less, or about 10⁻⁶ to about 10⁻⁸ s⁻¹ or less. In some embodiments, the binding agent has a k_{off} dissociation rate constant of about 10⁻³ s⁻¹ or less, about 10⁻⁴ s⁻¹ or less, about 10⁻⁵ s⁻¹ or less, about 10⁻⁶ s⁻¹ or less, about 10⁻⁷ s⁻¹ or less, about 10⁻⁸ s⁻¹ or less, about 10⁻⁹ s⁻¹ or less or about 10⁻¹⁰ s⁻¹ or less. In some embodiments, the binding agent comprises an antibody that binds specifically to MICA, MICB, ULBP1, ULBP2, ULBP3, ULBP4, ULBP5 or ULBP6, particularly MICA or MICB. In some embodiments, the antibody has a k_{off} dissociation rate constant characteristic of the antibody 1F5 or antibody 8C7 described herein.

In some embodiments, the K_{A} or K_{D} as well as the kₒₙ and k_{off} rate constants can be determined by surface plasmon resonance (SPR) screening, such as by analysis with a BIAcore™ SPR analytical device, as described in Popov et al., 1996, Mol Immunol. 33:493-502 and Karlsson et al., 1991, J Immunol. Methods 145:229-20, incorporated herein by reference. In some embodiments, the K_{A} or K_{D} as well as the kₒₙ and k_{off}rate constants can be determined by Bio-Layer Interferometry (BLI), which is based on interference pattern of white light reflected from two surfaces (see, e.g., Rich and Myszka, 2007, Anal Biochem. 361:1-6; Fransson et al., 2010, J Mol Biol. 398(2):214-31) and commercially available as Octet RED96 (ForteBio, Menlo Park, Calif., USA). Other methods for determining affinity and kinetic parameters include equilibrium dialysis and globulin precipitation (see, e.g., Azimzadeh et al., 1990, J Mol Recognit. 3(3):108-16).

In some embodiments, the binding agent comprises an antibody that binds specifically to the extracellular domain of MICA and/or MICB. Such antibodies should also bind specifically to sMICA and/or sMICB proteins. In some embodiments, the antibody (e.g., a polyclonal) can be directed to the full length MICA and/or MICB proteins, which should contain antibodies that bind specifically to the extracellular domain of MICA and/or MICB. In some embodiments, the antibody binds specifically to the alpha-1 domain, alpha-2 domain, and/or alpha-3 domain of the sMICA or sMICB protein.

Exemplary antibodies that bind specifically to the extracellular domain of MICA and/or MICB comprise antibodies described in U.S. Pat. No. 7,771,718 and International Patent Publication No. WO 03/089616, including monoclonal antibodies designated 6D4 and 6G6, which have been determined to bind an epitope on the alpha-1, alpha-2 and/or alpha-3 domains. Other useful antibodies include those described in Hue et al., 2003, J Immunol. 171:1909-17 and Hue, et al., 2004, Immunity 21:367-77, including monoclonal antibodies designated SR99, SR104 and SR116, which were selected for binding to MICA protein expressed on surface of cells. U.S. Pat. No. 8,182,809 describes an antibody that binds to the epitope NGTYQT (SEQ ID NO:61) located at amino acid residues 238 to 243 of the processed MICA protein, and is a putative binding site for the disulfide isomerase ERp5 involved in the proteolytic processing event that generates sMICA and sMICB in disease cells.

In some embodiments, exemplary antibodies that bind specifically to the extracellular domain of MICA and/or MICB are antibodies described in International Patent Publication WO2013117647, incorporated herein by reference. The antibodies bind to one or more MICA alleles from each of two major MICA groups: Group 1 alleles, which bind NKG2D strongly and include MICA alleles *001, *002, *007, *012, *017 and *018, and Group 2 alleles, which bind NKG2D weakly and include MICA alleles*004, *006, *008, *009 and *019. The antibodies of WO2013117647 can bind specifically to certain epitopes contained within amino acid residues 1-88, amino acid residues 86-181, or amino acid residues 182-274 of the MICA alleles. In some embodiments, the antibody can bind to an epitope on MICA comprising one or more amino acid residues selected from R6, N8, Q48, W49, E51, D52, V53, L54, N56, K57, T58, R61, R64, K81, D82, Q83, K84, E97, H99, E100, D101, N102, S103, T104, R105, H109, Y111, D113, E115, L116, N121, E123, T124, E126, Q131, S132, S133, R134, Q136, T137, M140, N141, R143, N144, L178, R179, R180, S224, H225, D226, T227, Q228, Q229, W230, and D232 of MICA, where the residue position is based on the mature, processed MICA protein encoded by the MICA *001 allele.

In some embodiments, the antibody binds an epitope on MICA comprising one or more amino acid residues selected from Q48, W49, E51, D52, V53 and L54. In some embodiments, the antibody binds an epitope comprising one or more amino acid residues selected from N56, K57, T58, R61 and R64. In some embodiments, the antibody binds an epitope comprising one or more amino acid residues selected from K81, D82, Q83, K84, H109, Y111, D113, L116, 8133, R134, T137, M140, N141, R143 and N144. In some embodiments, the antibody binds an epitope comprising one or more amino acid residues selected from K81, D82, Q83, K84, H109, Y111, D113, L116, Q131, S132, Q136, M140, N141, R143 and N144. In some embodiments, the antibody binds an epitope comprising one or more amino acid residues selected from E100, D101, N102, S103, T104, R105, N121, E123, T124 and E126. In some embodiments, the antibody binds an epitope comprising one or more amino acid residues selected from R6, N8, E97, H99, E100, D101, N102, S103, T104, R105, E115, L178, R179, and R180. In some embodiments, the antibody binds an epitope comprising one or more amino acid residues selected from S224, H225, D226, T227, Q228, Q229, W230, and D232. In some embodiments, the antibody binds an epitope comprising one or more amino acid residues selected from T227, Q228, and Q229.

While the amino acid residues at the foregoing specified residue positions are described with respect to the MICA 001 allele, it is to be understood that antibodies can bind to epitopes having different amino acid residues than those specified above. In some embodiments, the antibodies can bind to epitopes of MICA having one or more of the following amino acid substitutions selected from R6A, NBA, W14A, Q48A, W49S, E51S, D52A, V53S, L54A, N56A, K57S, T58A, R61A, R64A, K81A, DS2A, Q83A, K84A, E85A, E97A, H99A, E100A, D101S, N102A, S103A, T104S, R105A, H109A, Y111A, D113A, E115A, L116A,N121A, E123S, T124A, E126A, Q131A, S132A, S133A, R134S, Q136S, T137A, M140S, N141A, R143S, N144A, L178A, R179S, R180A, S224A, H225S, D226A, T227A, Q228S, Q229A, W230A, and D232A.

In some embodiments, the antibodies can comprise one or more of monoclonal antibodies selected from 6E4, 2006, 16A8, 9C10, 19E9, 12A10, 10A7, 18E8, 10F3, 15F9 and 14B4 described in WO2013117647. In some embodiments, the antibodies can comprise chimeric or humanized antibodies containing the CDR sequences of monoclonal antibodies selected from 6E4, 2006, 16A8, 9C10, 19E9, 12A10, 10A7, 18E8, 10F3, 15F9 and 14B4.

In some embodiments, the antibody that binds specifically to the extracellular domain of MICA and/or MICB can be obtained commercially. Such antibodies are available from Abcam (catalog nos. ab61282 and ab137847) (Cambridge, Mass., USA); Thermo Scientific (catalog no. PA5-28181) (Waltham, Mass., USA); Novus Biologicals (catalog no. NBP1-32830) (Oakville, ON, Canada); GeneTex (catalog nos. GTX105052 and GTX113495) (Irvine, Calif., USA); LifeSpan Biosciences (catalog no. LS-C164186-100) (Seattle, Wash., USA); Fitzgerald Industries International (catalog no. 70R-6091) (North Acton, Mass., USA); Sigma Aldrich (catalog nos. SAB1100065 and SAB1100064) (Oakville, ON, Canada); Bioss Inc. (catalog no. bs-0832R) (Woburn, Mass., USA); and GenWay Biotech (catalog no. GWB-MP766A) (San Diego, Calif., USA).

In some embodiments, the antibody binds specifically to sMICA and/or sMICB but does not bind specifically to full length MICA and/or MICB or extracellular domain of membrane bound form of MICA and/or MICB. In some embodiments, the antibody binds specifically to the alpha-3 domain of MICA and/or MICB but does not bind specifically to full length MICA and/or MICB or extracellular domain of membrane bound form of MICA and/or MICB. In some embodiments, the antibody binds specifically to cryptic epitopes on the alpha-3 domain of MICA and/or MICB proteins. These cryptic epitopes can be exposed by cell surface-localized proteolytic processing of the MIC protein such that antibodies capable of recognizing these epitopes can distinguish between soluble forms of MIC protein (or the extracellular domain of the MIC proteins) from the intact or membrane bound forms of MICA and/or MICB. In some embodiments, the antibodies bind specifically to a cryptic epitope on the alpha-3 domain, which epitope is within a polypeptide defined by an amino acid sequence from amino acid residues 187 to 296 or 187 to 297, particularly amino acid residues 187 to 274, more particularly amino acid residues 190 to 256 of MICA or MICB, where amino acid numbering is based on the processed MICA protein of the MICA*001 allele or the processed MICB protein of the MICB*001 allele, respectively.

In some embodiments, the cryptic epitopes are within a subsequence of the alpha-3 domain, wherein the subsequence is selected from:
amino acid residues 190 to 229;
amino acid residues 190 to 238;
amino acid residues 217 to 238;
amino acid residues 243 to 256;
amino acid residues 243 to 274; and
amino acid residues 243 to 296/297
of MICA or MICB.

In some embodiments, the cryptic epitope within the alpha-3 domain comprises a region selected from:
amino acid residues 190 to 196;
amino acid residues 217 to 221;
amino acid residues 234 to 238;
amino acid residues 250 to 256; and
amino acid residues 251 to 256
   of MICA, defined with respect to the MICA*001 allele, including the corresponding region in any of the alleles of MICA existing in the human population, such as the identified MICA alleles available in Robinson et al., 2003, “IMGT/HLA and IMGT/MHC: Sequence databases for the study of the major histocompatibility complex”, Nucleic Acids Res. 31:311-314 and the Anthony Nolan Research Institute world wide web site www.anthonynolan.org.uk/HIG/data.html; which are incorporated herein by reference. Thus, it is to be understood that for each and every embodiment of MICA cryptic epitopes described herein, the equivalent epitopes are also described for each and every one of the MICA allelic variants, including human MICA allelic variants selected from MICA*001, MICA*002:01, MICA*002:02, MICA*002:03, MICA*002:04, MICA*004, MICA*005, MICA*006, MICA*007:01, MICA*007:02, MICA*007:03, MICA*007:04, MICA*007:05, MICA*007:06, MICA*008:01:01, MICA*008:01:02, MICA*008:02, MICA*008:03, MICA*008:04, MICA*008:05, MICA*009:01, MICA*009:02, MICA*010:01, MICA*010:02, MICA*011, MICA*012:01, MICA*012:02, MICA*012:03, MICA*012:04, MICA*013, MICA*014, MICA*015, MICA*016, MICA*017, MICA*018:01, MICA*018:02, MICA*019, MICA*020, MICA*022, MICA*023, MICA*024, MICA*025, MICA*026, MICA*027, MICA*028, MICA*029, MICA*030, MICA*031, MICA*032, MICA*033, MICA*034, MICA*035, MICA*036, MICA*037, MICA*038, MICA*039, MICA*040, MICA*041, MICA*042, MICA*043, MICA*044, MICA*045, MICA*046, MICA*047, MICA*048, MICA*049, MICA*050, MICA*051, MICA*052, MICA*053, MICA*054, MICA*055, MICA*056, MICA*057, MICA*058, MICA*059, MICA*060, MICA*061, MICA*062, MICA*064N, MICA*065, MICA*066, MICA*067, MICA*068, MICA*069, MICA*070, MICA*072, MICA*073, MICA*074, MICA*075, MICA*076, and MICA*077.

In some embodiments, the cryptic epitope within the alpha-3 domain comprises a region selected from:
amino acid residues 190 to 196;
amino acid residues 217 to 221;
amino acid residues 234 to 238; and
amino acid residues 250 to 256
   of MICB, including the region in any of the alleles of MICB existing in the human population, such as the identified MICB alleles available in Robinson et al., 2003, “IMGT/HLA and IMGT/MHC: Sequence databases for the study of the major histocompatibility complex”, Nucleic Acids Res. 31:311-314 and the Anthony Nolan Research Institute world wide web site www.anthonynolan.org.uk/HIG/data.html; which are incorporated herein by reference. Thus, it is to be understood that for each and every embodiment of MICB cryptic epitopes described herein, the equivalent epitopes are also described for each and every one of the MICB allelic variants, including human MICB allelic variants selected from MICB*001, MICB*002:01:01, MICB*002:01:02, MICB*003, MICB*004:01:01, MICB*004:01:02, MICB*005:01, MICB*005:02:01, MICB*005:02:02, MICB*005:02:03, MICB*005:02:04, MICB*005:03, MICB*005:04, MICB*005:05, MICB*005:06, MICB*005:07, MICB*005:08, MICB*006, MICB*007, MICB*008, MICB*009N, MICB*010, MICB*011, MICB*012, MICB*013, MICB*014, MICB*015, MICB*016, MICB*018, MICB*019, MICB*020, MICB*021N, MICB *022, MICB*023, MICB*024, MICB*025, MICB*026, MICB*027, MICB*028, and MICB*029.

In some embodiments, the antibodies bind specifically to an epitope of MICA within the sequence defined by:
190_RSEASEG_196, located on bottom of alpha-3 domain (SEQ ID NO:24);
217_RQDGV_221, located on lower side of alpha-3 domain (SEQ ID NO:25);
234_LPDGN_238, located near the top of alpha-3 domain (SEQ ID NO:26);
251_QGEEQR256, located on bottom of alpha-3 domain (SEQ ID NO:27); or
251_RGEEQR_256, located on bottom of alpha-3 domain (SEQ ID NO:28),
   where the amino acid positions are defined with respect to the mature, processed MICA protein of the MICA*001 allele.

In some embodiments, the antibody binds an epitope comprising one or more amino acid residues selected from R190, S191, E192, A193, S194, E195, and G196, located on the bottom of the alpha-3 domain, where the amino acid positions are defined with respect to mature, processed MICA protein of the MICA*001 allele. In some embodiments, the epitope comprises 1, 2, 3 or more, or 1, 2, 3, 4 or more of the foregoing amino acid residues in the alpha-3 domain.

In some embodiments, the antibody binds an epitope comprising one or more amino acid residues selected from R217, Q218, D219, G220, and V221, located on the lower side of the alpha-3 domain, where the amino acid positions are defined with respect to mature, processed MICA protein of the MICA*001 allele. In some embodiments, the epitope comprises 1, 2, 3 or more, or 1, 2, 3, 4 or more of the foregoing amino acid residues in the alpha-3 domain.

In some embodiments, the antibody binds an epitope comprising one or more amino acid residues selected from Q251/R251, G252, E253, E254, Q255, and R256, located on the bottom of the alpha-3 domain, where the amino acid positions are defined with respect to mature, processed MICA protein of the MICA*001 allele. In some embodiments, the epitope comprises 1, 2, 3 or more, or 1, 2, 3, 4 or more of the foregoing amino acid residues in the alpha-3 domain.

In some embodiments, the antibody binds an epitope comprising one or more amino acid residues selected from L234, P235, D236, G237, and N238, located near the top of the alpha-3 domain, where the amino acid positions are defined with respect to mature, processed MICA protein of the MICA*001 allele. In some embodiments, the epitope comprises 1, 2, 3 or more, or 1, 2, 3, 4 or more of the foregoing amino acid residues in the alpha-3 domain.

In some embodiments, the isolated antibody binds specifically to an epitope of MICB within the sequence defined by:
190_CSEVSEG_196, located on bottom of alpha-3 domain (SEQ ID NO:29);
217_RQDGV_221, located on lower side of alpha-3 domain (SEQ ID NO:30);
234_LPDGN_238, located near the top of alpha-3 domain (SEQ ID NO:31); or
250_RQGEEQR_256, located on bottom of alpha-3 domain (SEQ ID NO:32),
   where the amino acid positions are defined with respect to the mature, processed MICB protein of the MICB*001 allele.

In some embodiments, the antibody binds an epitope comprising one or more amino acid residues selected from C190, S191, E192, V193, S194, E195, and G196, located on the bottom of the alpha-3 domain, where the amino acid positions are defined with respect to the mature, processed MICB protein of the MICB*001 allele. In some embodiments, the epitope comprises 1, 2, 3 or more, or 1, 2, 3, 4 or more of the foregoing amino acid residues in the alpha-3 domain.

In some embodiments, the antibody binds an epitope comprising one or more amino acid residues selected from R217, Q218, D219, G220, and V221, located on the lower side of the alpha-3 domain;, where the amino acid positions are defined with respect to the mature, processed MICB protein of the MICB*001 allele. In some embodiments, the epitope comprises 1, 2, 3 or more, or 1, 2, 3, 4 or more of the foregoing amino acid residues in the alpha-3 domain.

In some embodiments, the antibody binds an epitope comprising one or more amino acid residues selected from R250, Q251, G252, E253, E254, Q255, and R256, located on the bottom of the alpha-3 domain, where the amino acid positions are defined with respect to the mature, processed MICB protein of the MICB*001 allele. In some embodiments, the epitope comprises 1, 2, 3 or more, or 1, 2, 3, 4 or more of the foregoing amino acid residues in the alpha-3 domain.

In some embodiments, the antibody binds an epitope comprising one or more amino acid residues selected from L234, P235, D236, G237, and N238, located near the top of the alpha-3 domain, where the amino acid positions are defined with respect to the mature, processed MICB protein of the MICB*001 allele. In some embodiments, the epitope comprises 1, 2, 3 or more, or 1, 2, 3, 4 or more of the foregoing amino acid residues in the alpha-3 domain.

In some embodiments, the antibody binds specifically to an epitope of the alpha-3 domain within the amino acid sequence:
(a) ˜X^{A1}-S-X^{A3}-X^{A4}-S-E-G˜(SEQ ID NO:33),
   wherein X^{A1} is selected from R and C; X^{A3} is selected from E and K; and X^{A4} is selected from A and V;
(b) ˜R-Q-D-G-X^{B5}˜(SEQ ID NO:34),
   wherein X^{B5} is selected from V and L;
(c) ˜X^{D1}-X^{D2}-G-E-E-Q-X^{D7}˜(SEQ ID NO:35),
   wherein X^{D1} is selected from C or R; X^{D2} is selected from Q, R and E; and X^{D7} is selected from R, S and K; or
(d) ˜L-P-D-G-N˜(SEQ ID NO:36).

In some embodiments, the antibody for use with the embodiments herein can be antibodies described in PCT application entitled “Antibodies to MICA and MICB Proteins,” filed Mar. 15, 2014 (Docket No. NBI-001PCT), PCT application No. ______, incorporated herein by reference in its entirety, particularly the description of antibodies designated 1F5 and 8C7. The antibodies bind specifically to the alpha-3 domain of MICA but do not bind specifically to membrane bound MICA (e.g., MICA expressed on cell surface). Accordingly in some embodiments, the antibody for use in the embodiments herein has the antigen binding characteristics of antibody 1F5 or antibody 8C7. In some embodiments, the antibody comprises a CDR L1, CDR L2, and CDR L3 in the light chain variable region amino acid sequence comprising:

(SEQ ID NO: 37)
DIVLTQSPASLAVSLGQRATISCRASKSVSTSGYSYMHWYQQKPGQPPKL
LIYRASNLESGVPARFSGSGSGTDFTLNIHPVEEEDAATYYCQHSRELPL
TFGAGTKLELKR;

and a CDR H1, CDR H2 and CDR H3 in the heavy chain variable region amino acid sequence comprising:

(SEQ ID NO: 38)
QIQLVQSGPELKKPGETVKISCKASGYTFTDYSVHWVKQAPGKGLKWMGW
INTETGEPTYADDFKGRFAFSLETSASTAYLQINNLKNEDTATYFCARAG
GNAFAYWGQGTLVTVSA.

In some embodiments, the antibody comprises a CDR L1, CDR L2, and CDR L3 in the light chain variable region amino acid sequence comprising:

(SEQ ID NO: 39)
DIVMTQAAPSVPVTPGESVSISCRSSKSLLQSNGNTFLYWFMQRPGQSPQ
LLIYRMSNLASGVPDRFSGSGSGTAFTLRISRVEAEDVGVYYCMQHLEYP
FTFGGGTKLEIKR;

sand a CDR H1, CDR H2 and CDR H3 in the heavy chain variable region amino acid sequence comprising:

(SEQ ID NO: 40)
QIQLVQSGPELKKPGETVKISCKASGYTFTNYGMNWVKQAPGKGLKWMGW
INTNTGEPTYAEEFKGRFAFSLETSASTAYLQINNLKNEDTATYFCARSG
GSSPFAYWGQGTLVTVSA.

As is understood in the art and as described herein, the amino acid position/boundary delineating the CDR regions of an antibody can vary, depending on the context and the various definitions known in the art. Some positions within the variable regions can be viewed as hybrid CDRs in that the positions can be within a CDR region under one set of criteria while being deemed to be outside a CDR region under a different set of criteria. In some embodiments, the CDRs in the foregoing variable light and variable heavy chains can be delineated using the Kabat, Chothia, or AbM schemes, as described herein and known in the art, in particular using the foregoing schemes based on the Kabat numbering system. However, it is to be understood that CDRs based on other methods, including the “Contact” approach, IMGT approach (Lefranc et al., 2003, Dev Comp Immunol. 27(1):55-77) and computational programs such as Paratome (Kunik et al., 2012, Nucl Acids Res. W521-4; www.ofranlab.org/paratome/) are to be encompassed herein.

In some embodiments, the antibody comprises a CDR L1 comprising an amino acid sequence RASKSVSTSGYSYMH (SEQ ID NO:41); a CDR L2 comprising an amino acid sequence RASNLES (SEQ ID NO:42); a CDR L3 comprising an amino acid sequence QHSRELPLT (SEQ ID NO:43); a CDR H1 comprising an amino acid sequence DYSVH (SEQ ID NO:44), GYTFTDY (SEQ ID NO:45), or GYTFTDYSVH (SEQ ID NO:46); a CDR H2 comprising an amino acid sequence WINTETGEPTYADDFKG (SEQ ID NO:47), NTETG (SEQ ID NO:48), or WINTETGEP (SEQ ID NO:49); and a CDR H3 comprising an amino acid sequence AGGNAFAY (SEQ ID NO:50).

In some embodiments, the antibody comprises a CDR L1 comprising an amino acid sequence RASKSVSTSGYSYMH (SEQ ID NO:41); a CDR L2 comprising an amino acid sequence RASNLES (SEQ ID NO:42); a CDR L3 comprising an amino acid sequence QHSRELPLT (SEQ ID NO:43); a CDR H1 comprising an amino acid sequence DYSVH (SEQ ID NO:44); a CDR H2 comprising an amino acid sequence WINTETGEPTYADDFKG (SEQ ID NO:47); and a CDR H3 comprising an amino acid sequence AGGNAFAY (SEQ ID NO:50).

In some embodiments, the antibody comprises a CDR L1 comprising an amino acid sequence RSSKSLLQSNGNTFLY (SEQ ID NO:51); a CDR L2 comprising an amino acid sequence RMSNLAS (SEQ ID NO:52); a CDR L3 comprising an amino acid sequence MQHLEYPFT (SEQ ID NO:53); a CDR H1 comprising an amino acid sequence NYGMN (SEQ ID NO:54), GYTFTNY (SEQ ID NO:55), or GYTFTNYGMN (SEQ ID NO:56); a CDR H2 comprising an amino acid sequence WINTNTGEPTYAEEFKG (SEQ ID NO:57), NTNTG (SEQ ID NO:58), or WINTNTGEP (SEQ ID NO:59); and a CDR H3 comprising an amino acid sequence SGGSSPFAY (SEQ ID NO:60).

In some embodiments, the antibody comprises a CDR L1 comprising an amino acid sequence RSSKSLLQSNGNTFLY (SEQ ID NO:51); a CDR L2 comprising an amino acid sequence RMSNLAS (SEQ ID NO:52); a CDR L3 comprising an amino acid sequence MQHLEYPFT (SEQ ID NO:53); a CDR H1 comprising an amino acid sequence NYGMN (SEQ ID NO:54); a CDR H2 comprising an amino acid sequence WINTNTGEPTYAEEFKG (SEQ ID NO:57); and a CDR H3 comprising an amino acid sequence SGGSSPFAY (SEQ ID NO:60).

In some embodiments, the antibody comprises a light chain variable region VL comprising an amino acid sequence of SEQ ID NO:37 and a heavy chain variable region VH comprising an amino acid sequence of SEQ ID NO:38.

In some embodiments, the antibody comprises a light chain variable region VL comprising an amino acid sequence of SEQ ID NO:39 and a heavy chain variable region VH comprising an amino acid sequence of SEQ ID NO:40.

In some embodiments, the antibody with any of the specified antigen binding domains can comprise any suitable framework variable region sequence, provided the functional properties of the antigen binding domain in binding to sMICA and/or sMICB, or the alpha-3 domain thereof, are maintained. In some embodiments, the framework sequences are those of rodent variable light chain and variable heavy chain framework sequences, in particular mouse framework sequences. In some embodiments, the framework sequences of the antibody are those of a human consensus framework sequence. Exemplary human consensus framework regions include, human VH subgroup I consensus framework; human VH subgroup II consensus framework; human VH subgroup III consensus framework; human VH subgroup VII consensus framework; human VL subgroup I consensus framework; human VL subgroup II consensus framework; human VL subgroup III consensus framework; and human VL subgroup IV consensus framework (see, e.g., U.S. patent publication no. 2012/0230985, incorporated herein by reference).

In some embodiments, the antibody with any of the specified antigen binding domains can have a constant domain of any origin on the light chain and/or the heavy chain. The constant domain can be that of rodent, primate, or other mammals. In some embodiments, the constant domain is of rodent origin, particularly mouse. In some embodiments, the constant domain is of human origin. Accordingly, in some embodiments, the antibody with any of the specified antigen binding domains above can have a human constant region, for example, a human light chain constant region CL and/or a human heavy chain constant region. In some embodiments, the human light chain constant region CL comprises a human kappa or human lambda constant region. In some embodiments, the human heavy chain constant region comprises at least one or all of the following: a human CH1, human Hinge, human CH2 and human CH3 domain. In some embodiments, the heavy chain constant region comprises an Fc portion, where the Fc portion is a human IgG₁, IgG₂, IgG₃, IgG₄ or IgM isotype.

In some embodiments, an exemplary antibody for use in the embodiments herein comprises a light chain variable region VL comprising an amino acid sequence of SEQ ID NO:37; a heavy chain variable region VH comprising an amino acid sequence of SEQ ID NO:38; a human light chain constant (CL) region of human kappa or lambda; and a human heavy chain constant region, in particular a human heavy chain constant region comprising human CH1, human Hinge, human CH2 and human CH3 domains. In some embodiments, the heavy chain constant region comprises an Fc portion, where the Fc portion is a human IgG₁, IgG₂, IgG₃, IgG₄ or IgM isotype.

In some embodiments, another exemplary antibody for use in the embodiments herein comprises a light chain variable region VL comprising an amino acid sequence of SEQ ID NO:39; a heavy chain variable region VH comprising an amino acid sequence of SEQ ID NO:40; a human light chain constant (CL) region of human kappa or lambda; and a human heavy chain constant region, in particular a human heavy chain constant region comprising human CH1, human Hinge, human CH2 and human CH3 domains. In some embodiments, the heavy chain constant region comprises an Fc portion, where the Fc portion is a human IgG₁, IgG₂, IgG₃, IgG₄ or IgM isotype.

In some embodiments, the binding agent comprises an antibody capable of binding specifically to the extracellular domain of ULBP1. Such antibodies should also bind specifically to sULBP1 protein. In some embodiments, the antibody (e.g., polyclonal, monoclonal, etc.) can be directed to the full length ULBP1 protein, or in some embodiments, an antibody that binds specifically to the alpha-1 domain or alpha-2 domain of the sULBP1 protein.

In some embodiments, the binding agent comprises an antibody that binds specifically to the extracellular domain of ULBP2. Such antibodies should also bind specifically to sULBP2 protein. In some embodiments, the antibody (e.g., polyclonal, monoclonal, etc.) can be directed to the full length ULBP2 protein or in some embodiments, an antibody that binds specifically to the alpha-1 domain or alpha-2 domain of the sULBP2 protein.

In some embodiments, the binding agent comprises an antibody that binds specifically to the extracellular domain of ULBP3. Such antibodies should also bind specifically to sULBP3 protein. In some embodiments, the antibody (e.g., polyclonal, monoclonal, etc.) can be directed to the full length ULBP3 protein or in some embodiments, an antibody that binds specifically to the alpha-1 domain or alpha-2 domain of the sULBP3 protein.

In some embodiments, the binding agent comprises an antibody that binds specifically to the extracellular domain of ULBP4. Such antibodies should also bind specifically to sULBP4 protein. In some embodiments, the antibody (e.g., polyclonal, monoclonal, etc.) can be directed to the full length ULBP4 protein or in some embodiments, an antibody that binds specifically to the alpha-1 domain or alpha-2 domain of the sULBP4 protein.

In some embodiments, the binding agent comprises an antibody that binds specifically to the extracellular domain of ULBP5. Such antibodies should also bind specifically to sULBP5 protein. In some embodiments, the antibody (e.g., polyclonal, monoclonal, etc.) can be directed to the full length ULBP5 protein or in some embodiments, an antibody that binds specifically to the alpha-1 domain or alpha-2 domain of the sULBP5 protein.

In some embodiments, the binding agent comprises an antibody that binds specifically to the extracellular domain of ULBP6. Such antibodies should also bind specifically to sULBP6 protein. In some embodiments, the antibody (e.g., polyclonal, monoclonal, etc.) can be directed to the full length ULBP6 protein or in some embodiments, an antibody that binds specifically to the alpha-1 domain or alpha-2 domain of the sULBP6 protein.

In some embodiments, the binding agent comprises an antibody that binds two or more ULBP proteins, particularly given the level of conserved regions between some ULBP proteins. For example, in some embodiments, the antibody binds specifically to the extracellular domain of ULBP2 and ULPB3, such as the alpha-1 domain or alpha-2 domain of the sULBP2 and sULBP3 protein.

Exemplary antibodies that bind specifically to sULBP1 protein are described in patent publication no. US20080008715, incorporated herein by reference. Antibodies that can bind specifically to sULBP1 can also be obtained from commercial sources, including from commercial suppliers Abcam (catalog no. ab90039; Cambridge, Mass., USA) and BAMOMAB (catalog no. AUMO2-100) (Grafelfmg, Germany).

Exemplary antibodies that bind specifically to sULBP2 protein are described in patent publication no. US20120295288, incorporated herein by reference. Antibodies that can bind specifically to ULBP2 can also be obtained from commercial sources, including from commercial suppliers Abcam (catalog nos. ab67186, ab88645, ab130591, and ab130482) (Cambridge, Mass., USA), Novus Biologicals (catalog no. 27080002) (Oakville, ON, Canada), and Santa Cruz Biotechnology (catalog no. sc-53135, sc-33565, and sc-53132) (Dallas, Tex., USA).

Exemplary antibodies that bind specifically to sULBP3 proteins are described in patent publication no. US20090324597, incorporated herein by reference. Antibodies that can bind specifically to ULBP3 can also be obtained from commercial sources, including from commercial suppliers Abcam (catalog no. ab130482) (Cambridge, Mass., USA) and Santa Cruz Biotechnology (catalog no. sc-53132—monoclonal 2F9) (Dallas, Tex., USA).

Exemplary antibodies that bind specifically to sULBP4 protein are described in patent publications nos. US20030195337, US20090274699 and U.S. Pat. No. 7,563,450, all of which are incorporated herein by reference. Antibodies that can bind specifically to ULBP4 can also be obtained from commercial sources, including from commercial suppliers Abcam (catalog no. ab95202) (Cambridge, Mass., USA) and Santa Cruz Biotechnology (catalog no. sc-55793 and sc-135180) (Dallas, Tex., USA).

Exemplary antibodies that bind specifically to sULBP5 protein are described in Ohashi et al., 2010, J Biol Chem. 285(22):16408-15, incorporated herein by reference. Antibodies that can bind specifically to ULBP4 can also be obtained from commercial sources, including from commercial suppliers Abcam (catalog no. ab169358 and ab166345) (Cambridge, Mass., USA) and Santa Cruz Biotechnology (catalog no. sc-53134-monoclonal 6D10) (Dallas, Tex., USA).

Exemplary antibodies that bind specifically to sULBP6 protein can employ antibodies to ULBP2 that are cross reactive with ULBP6. Such cross reactive antibodies are available commercially from R&D Systems (catalog no. FAB1298P and MAB1298) (Minneapolis, Minn., USA).

In some embodiments, the binding agent comprises two or more antibodies selected from an antibody that binds specifically to sMICA, an antibody that binds specifically to sMICB, an antibody that binds specifically to sULBP1, an antibody that binds specifically to sULBP2, an antibody that binds specifically to sULBP3, an antibody that binds specifically to sULBP4, an antibody that binds specifically to sULBP5, and an antibody that binds specifically to sULBP6.

In the embodiments herein, the antibodies can comprise any type of antibody suitable for the purposes herein, including monoclonal antibodies, polyclonal antibodies and multispecific antibodies. In some embodiments, the antibody can comprise a non-human antibody, such as prepared from goat, horse, cow, chicken, camel, llamas, rabbit, rat, or mouse, etc.; a chimeric antibody; a humanized antibody; a fully human antibody; or combinations thereof.

In some embodiments, the antibody that can be used comprises a multimeric antibody containing three or more binding sites, for example an IgM isotype or a synthetically generated multimeric antibody. IgM antibodies generally have four, five, or six units of bivalent binding units, i.e., two heavy chains and two light chains assembled into a tetramer, pentamer and/or hexamer. The IgM antibody may or may not have a J chain Expression of IgM without a J chain forms predominantly hexamers while expression of IgM with J chains forms predominantly pentamers. The multimeric antibodies would promote efficient binding to sMIC and/or sULBP proteins due in part to high avidity resulting from the higher number of antigen binding sites. In some embodiments, IgM antibodies can be obtained by isolating IgM antibodies from immunized animals, by isolating monoclonal antibody producing cell lines (e.g., hybridoma cell lines, etc.) expressing IgM isotype antibody, or transfection/transformation of appropriate cell lines (e.g., CHO, COS, 3T3, PC12, BHK, Vero, C6 glioma, and HeLa) with nucleic acids encoding an IgM antibody or IgM variable heavy and variable light chains, with or without J chains (see, e.g., Azuma et al., 2007, Clin Cancer Res. 13:2745-50; Mader et al., 2013, Adv Biosci Biotech. 4:38-43; U.S. Pat. No. 7,709,615). In some embodiments, an initially isolated IgG antibody can be class switched to the IgM isotype by expression in appropriate cells lines. For example, Kunert et al., 2004, AIDS Res Human Retroviruses 20:755-62 and Wolbank et al., 2003, J Virol. 77:4095-103 describes switching of IgG monoclonal antibodies to IgM isotype. In some embodiments, multimeric antibodies can be generated using single chain antibodies or antibody fragments produced as multimeric antibodies (see, e.g., Power et al., 2003, Methods Mol Biol. 207:335-50; Gail et al., 1999, FEBS Lett. 453(1-2):164-8). The IgM or single chain multimeric antibodies can be purified by techniques known in the art, such as gel filtration chromatography, ion exchange chromatography (e.g., hydroxylapatite), and affinity chromatography (see, e.g., Valasek et al., 2011, BioProcess Intl. 9(11):28-37; Gagnon et al., 2008, BioPharm Intl. S26-S36). In some embodiments, the multimeric antibodies can comprise 50% or more hexamer, 60% or more pentamer, or particularly 80% or more pentamer or hexamer IgM molecule. In some embodiments, the IgM or multimeric antibody comprises an antibody binding region of antibody 1F5 or 8C7 described herein.

In some embodiments, the binding agent can be a functional fragment of any of the antibody described above, including portions of the full length antibody, and includes the antigen binding or variable region. Exemplary antibody fragments include Fab, Fab′, F(ab′)2 and Fv fragments. Proteolytic digestion with papain produces two identical antigen binding fragments, the Fab fragment, each with a single antigen binding site. Proteolytic digestion with pepsin yields an F(ab′)2 fragment that has two antigen binding fragments which are capable of cross-linking antigen, and a residual pFc′ fragment. Other types of fragments include diabodies, linear antibodies, single-chain antibody molecules, and multispecific antibodies formed from antibody fragments. As noted above, the antibody fragments are functional in that they retain the desired binding properties, e.g., specific binding to sMICA, sMICB, sULBP1, sULBP2, sULBP3, sULBP4, sULBP5, or sULBP6; or specific binding to cryptic epitopes in the alpha-3 domain of MICA and/or MICB.

Other antibodies that bind specifically to the extracellular domain can be made using the extracellular domain of MICA (e.g., MICA and MICB) and ULBP proteins, (e.g., ULBP1, ULBP2, ULBP3, ULB4, ULBP5, and ULBP6) and polypeptide fragments thereof, as an immunogen. The preparation of polyclonal antibodies can employ conventional procedures well-known to those skilled in the art, for example, Green, et al., “Production of Polyclonal Antisera,” in Immunochemical Protocols, Manson, ed., Humana Press (1992); Coligan, et al., “Production of Polyclonal Antisera in Rabbits, Rats Mice and Hamsters,” in Current Protocols in Immunology, section 2.4.1, John Wiley & Sons, Inc. (1992); which publications are hereby incorporated herein by reference.

The preparation of monoclonal antibodies can also use conventional techniques known in the art, for example, Kohler and Milstein, 1975, Nature 256(5517):495-7; Coligan et al., supra, sections 2.5.1-2.6.7; Current Protocols in Immunology, John Wiley & Sons, Inc. (1992); and Antibodies: A Laboratory Manual, Harlow and Lane eds., Cold Spring Harbor Press (1988); Monoclonal Antibodies: Methods and Protocols in Methods Mol Biol. Vol. 378, Albitar M., ed., Humana Press (2007), which are hereby incorporated herein by reference. Monoclonal antibodies are most frequently generated in mice by administration of the “antigen” and subsequent isolation of B-cells that make antibodies. The B-cells are then immortalized by fusion to another, stable cell type of the same species of the B-cell to create a “hybridoma”. An individual B-cell makes one specific antibody (i.e., is clonally monospecific) which is defined by its primary amino acid sequence and its underlying gene sequence. Also, the terms “heterohybridoma” and “heteromyeloma” refer to lymphocyte cell lines immortalized by fusion of lymphocytes and myelomas from two different species. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., supra, sections 2.7.1-2.7.12 and sections 2.9.1-2.9.3; Barnes et al., Purification of Immunoglobulin G (IgG), in Methods Mol Biol., Vol. 10, pages 79-104, Humana Press (1992).

In some embodiments, the generation of monoclonal antibodies can be achieved using immunogens derived from DNA, peptides, or proteins. Hybridomas are generated by immunizing an animal, which can be for example, a mouse or rabbit, or any animal that will give a suitable antibody response. In some embodiments, immunization is performed by introducing into the animal an antigen-encoding nucleic acid, or a protein antigen, such as MICA, MICB, ULBP1, ULBP2, ULBP3, ULBP4, ULBP5, or ULBP6, or a fragment thereof, or a nucleic acid encoding MICA, MICB, ULBP1, ULBP2, ULBP3, ULBP4, ULBP5, or ULBP6, or an immunogenic fragment thereof. The skilled artisan will appreciate that certain epitopes will be more immunogenic in animals when removed from their native environment. Thus, a peptide corresponding to an epitope of an antigen conjugated to a carrier such as keyhole limpet hemocyanin, may elicit a stronger antibody response than either the peptide alone or the epitope when part of the native protein on which it is found. Such variations and other immunization schemes are known to the skilled artisan.

In some embodiments, the antibodies comprise chimeric antibodies, which have variable sequences derived from a non-human immunoglobulin (such as rat or mouse antibody) and human immunoglobulin constant regions, typically chosen from a human immunoglobulin template. One method for generating chimeric antibodies is to clone the non-human genes encoding the variable regions and the human genes encoding the constant regions and recombine them using recombinant techniques to form a chimeric gene. Expression in appropriate cells produces an mRNA encoding the chimeric protein. An alternative process is to use homologous recombination, where a rodent or mouse hybridoma cell line is transfected with a human constant region gene flanked by sequences homologous to the corresponding rodent immunoglobulin constant region gene. At a low frequency the transfected DNA will recombine with the rodent gene resulting in the insertion of the human immunoglobulin constant region gene sequence. Various methods for producing chimeric antibodies are described in Morrison et al., 1985, Science 229(4719):1202-7; Neuberger et al., 1985, Nature 314:268-71; Oi et al., 1986, BioTechniques 4:214-21; Gillies et al., 1985, J Immunol. Methods 125:191-202; U.S. Pat. No. 5,807,715; U.S. Pat. No. 4,816,567; and U.S. Pat. No. 4,816,397, all of which are incorporated herein by reference in their entireties.

In some embodiments, the antibodies herein can be prepared as humanized antibodies, which are chimeric immunoglobulins, immunoglobulin chains or fragments thereof (such as Fv, Fab, Fab′, F(ab′)2 or other target-binding sub domains of antibodies) which contain minimal sequences derived from non-human immunoglobulin. In general, the humanized antibody will comprise substantially all of at least one, and typically two, variable domains, in which all or substantially all of the complementarity determining regions (CDR) are those of a non-human immunoglobulin and all or substantially all of the framework (FR) regions are those of a human immunoglobulin sequence. The humanized antibody can also comprise at least a portion of an immunoglobulin constant region (Fc), typically that of a human immunoglobulin consensus sequence. Methods of antibody humanization are known in the art, and are described in various publications, for example, Riechmann et al., 1988, Nature 332:323-7; U.S. Pat. No. 5,225,539; U.S. Pat. No. 5,530,101; U.S. Pat. No. 5,585,089; U.S. Pat. No. 5,565,332; U.S. Pat. No. 5,693,761; U.S. Pat. No. 5,693,762; U.S. Pat. No. 6,180,370; PCT publication WO 91/09967; Padlan et al., 1991, Mol Immunol. 28:489-98; Studnicka et al., 1994, Prot Eng. 7:805-14; Roguska et al., 1994, Proc Natl Acad Sci USA. 91:969-73, all of which are hereby incorporated by reference in their entireties.

Fully human antibodies can be generated using transgenic or trans-chromosomic animals carrying parts of the human immune system rather than the host animal system. These transgenic and trans-chromosomic animals include mice referred to herein as HuMAb mice and KM mice. The HuMAb mouse™ (Medarex, Inc.) contains human immunoglobulin gene miniloci that encode un-rearranged human heavy (mu and gamma) and kappa light chain immunoglobulin sequences, together with targeted mutations that inactivate the endogenous mu and kappa chain loci (see, e.g., Lonberg et al., 1994, Nature 368(6474):856-9). Accordingly, the mice exhibit reduced expression of mouse IgM or kappa, and in response to immunization, the introduced human heavy and light chain transgenes undergo class switching and somatic mutation to generate high affinity human IgG kappa antibodies (Lonberg, N., 1994, Handbook of Experimental Pharmacology 113:49-101; Lonberg and Huszar, 1995, Intern Rev Immunol. 13:65-93; and Harding and Lonberg, 1995, Ann NY Acad Sci. 764:536-46). The preparation and use of HuMAb mice, and the genomic modifications carried by such mice, are further described in Tuaillon et al., 1994, J Immunol. 152:2912-20; Taylor et al., 1994, International Immunology 579-91; Fishwild et al., 1996 Nature Biotech.14:845-51; and patent publications U.S. Pat. No. 5,545,806; U.S. Pat. No. 5,569,825; U.S. Pat. No. 5,625,126; U.S. Pat. No. 5,633,425; U.S. Pat. No. 5,789,650; U.S. Pat. No. 5,877,397; U.S. Pat. No. 5,661,016; U.S. Pat. No. 5,814,318; U.S. Pat. No. 5,874,299; U.S. Pat. No. 5,770,429; U.S. Pat. No. 5,545,807; and PCT publications WO 92103918, WO 93/12227, WO 94/25585, WO 97113852, WO 98/24884, WO 99/45962, and WO 01/14424; the contents of all of which are hereby specifically incorporated herein in their entirety by reference. An alternative transgenic system referred to as the Xenomouse™ (Abgenix, Inc.) can be used, which are described in U.S. Pat. No. 5,939,598; U.S. Pat. No. 6,075,181; U.S. Pat. No. 6,114,598; U.S. Pat. No. 6,150,584; and U.S. Pat. No. 6,162,963.

In some embodiments, human antibodies that bind specifically to epitopes can be raised using a mouse that carries human immunoglobulin sequences on transgenes and trans-chromosomes, such as a mouse that carries a human heavy chain transgene and a human light chain trans-chromosome, as described in WO 02/043478. In some embodiments, a rabbit system expressing human immunoglobulin genes can be used to generate fully human antibodies (Rader et al., 2000, J Biol Chem. 275(18):13668-76).

In other embodiments, fully human monoclonal antibodies can be prepared using phage display methods for screening libraries of human immunoglobulin genes. Such phage display methods for isolating human antibodies are established in the art, and are described in, for example, Marks and Bradbury, 2004, Methods Mol Biol., 248:161-76; Pansri et al., BMC Biotech., 9:6-22; Rader, C., 2012, Methods Mol Biol., 901:53-79; U.S. Pat. No. 5,223,409; U.S. Pat. No. 5,403,484; U.S. Pat. No. 5,571,698; U.S. Pat. No. 5,427,908; U.S. Pat. No. 5,580,717; U.S. Pat. No. 5,969,108; U.S. Pat. No. 6,172,197; U.S. Pat. No. 5,885,793; U.S. Pat. No. 6,521,404; U.S. Pat. No. 6,544,731; U.S. Pat. No. 6,555,313; U.S. Pat. No. 6,582,915 and U.S. Pat. No. 6,593,081.

Single chain antibodies, which are fusion proteins of the variable heavy chains and variable light chains of immunoglobulins, can be prepared by phage display methods noted above, where the antigen binding domain is expressed as a single polypeptide and screened for specific binding activity. Alternatively, the single chain antibody can be prepared by cloning the heavy and light chains from a cell, typically a hybridoma cell line expressing a desired antibody. Generally, a linker peptide, typically from 10 to 25 amino acids in length is used to link the heavy and light chains. The linker can be glycine, serine, and/or threonine rich to impart flexibility and solubility to the single chain antibody. Specific methods for generating single chain antibodies are described in, for example, Loffler et al., 2000, Blood 95(6):2098-103; Worn and Pluckthun, 2001, J Mol Biol. 305, 989-1010; Pluckthun, In The Pharmacology of Monoclonal Antibodies, Vol. 113, pp. 269-315, Rosenburg and Moore eds. Springer-Verlag, New York (1994); U.S. Pat. No. 5,840,301; U.S. Pat. No. 5,844,093; and U.S. Pat. No. 5,892,020; all of which are incorporated herein by reference.

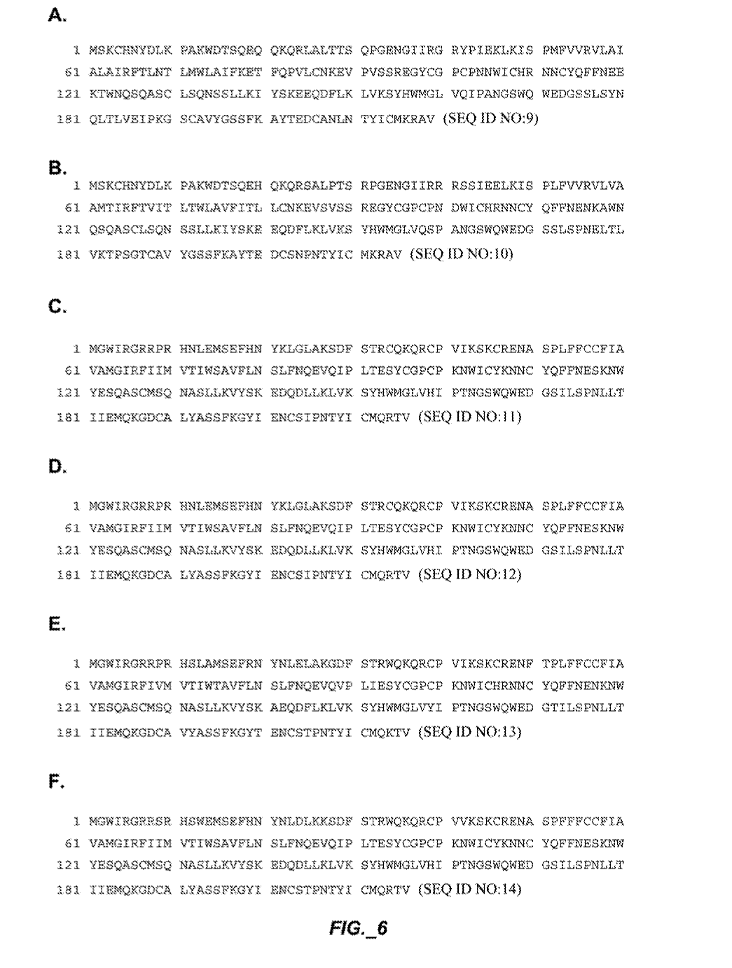

In some embodiments, the binding agent for use in the therapeutic methods comprises a receptor that can bind specifically to the soluble NKG2D ligand, e.g., sMICA, sMICB and/or sULBP proteins. In some embodiments, the receptor can comprise the NKG2D receptor or a functional NKG2D receptor fragment that retains the specific binding characteristics of the intact NKG2D receptor. The NKG2D receptor can be the human receptor, as presented on FIG. 5 (SEQ ID NO:7). In some embodiments, the NKG2D receptor can be variants or homologs of the human receptor, such as the corresponding NKG2D receptors presented in FIG. 6 (SEQ ID NOS:9-14). In some embodiments, the NKG2D receptor protein can be used intact, without modification. In some embodiments, the NKG2D receptor can be expressed as a fusion protein, such as a fusion to the Fc portion of an immunoglobulin, or a linker peptide that permits immobilization of the fusion protein to a solid carrier for carrying out the methods, systems, and devices of the disclosure.

In some embodiments, the receptor can comprise a functional human cytomegalovirus (HCMV) UL16 viral protein (FIG. 8; SEQ ID NO:21), functional HCMV UL142 viral protein (FIG. 9; SEQ ID NO:22), functional human herpes virus -7 (HHV-7) U21 viral protein (FIG. 10; SEQ ID NO:23), or combinations thereof. In some embodiments, the receptor can comprise a functional fragment of one or more of the foregoing proteins. These viral proteins function as immunoevasins that bind MIC and/or ULBP proteins (see, e.g., Muller et al., 2010, PLoS Pathog. 6(1): e1000723). Without being bound by theory, HCMV-UL16 and HCMV UL142 can selectively prevent the surface expression of MICB, ULBP1, ULBP2 (UL16) and/or MICA through intracellular retention of the relevant NKG2D ligand (see Dunn et al., 2003, J Exp Med. 197:1427-39; Welte et al., 2003, Eur J Immunol. 33:194-203; Chalupny et al., 2006, Biochem Biophys Res Commun 346:175-81).

In some embodiments, the receptor HCMV-UL16 can be used as a specific binding agent for MICB, ULBP1, and ULBP2 proteins (see Muller et al., supra). An exemplary amino acid sequence of HCMV-UL16 is presented in FIG. 8 (SEQ ID NO:21).

In some embodiments, receptor HCMV UL142 can be used as a specific binding agent for MICA and ULBP3. An exemplary amino acid sequence of HCMV UL142 protein is presented in FIG. 9 (SEQ ID NO:22).

In some embodiments, receptor HHV-7 U21 can be used as a specific binding agent for MICA and MICB (Schneider and Hudson, 2011, PLoS Pathog. 7(11):e1002362). An exemplary amino acid sequence of HHV-7 U21 is presented in FIG. 10 (SEQ ID NO:23).

In some embodiments, any one or more of the receptors for the soluble NKG2D ligands can be used in isolation or in combination with antibody binding agents. For example, a functional NKG2D receptor can be used in combination with one or more antibodies that bind specifically to sMICA, sMICB, sULBP1, sULBP2, sULBP3, sULBP4, sULBP5, and/or sULBP6. Where different binding agents are used, the binding agent can be a mixture of binding agents, for example, a mixture of solid carrier comprising a NKG2D receptor and a MICA/MICB antibody. In some embodiments, the binding agents can be used sequentially, for example, treatment of plasma with a first solid carrier comprising a NKG2D receptor and a second solid carrier comprising an antibody that binds specifically to sMICA, sMICB and/or a sULBP protein. In some embodiments of a device or system described further below, the first solid carrier can be contained in a first column and the second solid carrier contained in a second column, which columns can be independent of each other or be in fluid communication.

Generally, to facilitate treatment of the plasma or blood, and removal of the plasma fraction from the complexes formed between the binding agent and soluble NKG2D ligands, the binding agents are immobilized on a solid carrier. The solid carrier can be any substrate to which the binding agent can be immobilized, or a substrate that can be modified to permit immobilization of the binding agent onto the solid carrier. In some embodiments, the solid carrier can comprise agarose, dextran, polyacrylamide, silica, polysulfone, cellulose, polyamide, polyether, polyethylene, polypropylene, polyester, or derivatives or mixtures thereof. Particularly useful are crosslinked derivatives of agarose (e.g., Sepharose), or modified substrates of dextran, polyacrylamide, polysulfone, polyamide, polyvinyl, and polyethers. The solid carriers can be in any geometric or physical form for carrying out the methods, and includes forms such as particles, membranes, tubes, or channels (i.e., coils, fins, etc.) that can be contacted by the plasma or blood fraction. The solid carrier can comprise a single binding agent, or comprise a combination of two or more different binding agents, as described herein.

In some embodiments, where the solid carrier is in the form of a particle, the average particle diameter can be about 5 to about 1000 μm, particularly about 25 to about 1000 μm, or more particularly about 50 to about 300 μm, particularly when the plasma fraction is being treated. In some embodiments, the average particle diameter can be about 5 to about 1000 μm, particularly about 250 to about 1,000 μm, or more particularly about 250 to about 600 μm, particularly when whole blood is being treated. In some embodiments, particle size may also be varied depending on the anticoagulant used during treatment. For example, when citric acid is the anticoagulant, the average particle diameter can be about 5 to about 1000 μm, particularly about 100 to about 600 μm, or more particularly about 250 to about 300 μm. When heparin is the anticoagulant, the average particle diameter can be about 5 to about 1000 μm, particularly about 250 to about 1,000 μm, and more particularly about 350 to about 600 μm.

In some embodiments, the plasma fraction or blood passes through the solid carrier (such as a column, tube, or other container that can come in contact with the plasma fraction or blood), where the sNKG2D ligands can bind to the binding agents immobilized on the solid carrier, and the treated plasma or blood is separated from the complexes of bound soluble NKG2D ligand and binding agent. The interior walls of the column, tube or other type of containers can be configured to increase the surface area, such as having fins or folds on the inside of the container.

For the embodiments herein, the binding agent can be immobilized on the solid carrier using conventional methods available to the skilled artisan. In some embodiments, the binding agent can be attached to the solid carrier covalently by using reactive functional groups on the carrier and the binding agent (see, e.g., Bioconjugate Techniques, Greg T. Hermanson, ed., Academic Press Inc., San Diego, Calif. (1995)). For example, functional groups on antibodies useful for coupling to solid carriers include amino, carboxy, sulfhydryl, and hydroxy groups. The functional groups can be on the protein portion of an antibody, or in some embodiments, the carbohydrate moiety attached to the antibody. Functional groups on the solid carrier can be activated with coupling agents to facilitate reaction with functional groups on the binding agent. Coupling agents that can be used include, among others, glutaraldehyde, cyanogen bromide, p-benzoquinone, succinic anhydrides, carbodiimides, diisocyanates, ethyl chloroformate, periodate, dipyridyl disulphide, epichlorohydrin, azides, and the like. In some embodiments, the binding agent can be coupled to the solid carrier via bifunctional spacer linkers after modification with chemically reactive groups (e.g., amine, hydroxy, keto, sulfhydryl, and/or carboxyl). Examples of spacer backbones for suitable spacer linkers include, among others, substituted and unsubstituted C₂-C₁₀ alkyl groups, substituted and unsubstituted C₂-C₁₀ alkenyl groups, substituted or unsubstituted C₂-C₁₀ alkynyl groups, substituted and unsubstituted C₄-C₇ carbocycloalkyl groups, substituted and unsubstituted C₇-C₁₄ aralkyl groups, or a heterocyclic molecule with hetero atoms selected from nitrogen, oxygen, and sulfur. Substitutions may consist of alkyl, alkenyl, alkynyl, alkoxy, thiol, thioalkoxy, hydroxyl, aryl, benzyl, phenyl, nitro, halogen, ether groups with 2 to 10 carbon atoms and 1 to 4 oxygen- or sulfur atoms, polyalkylglycol, halogen, hydroxyl, thiol, keto, carboxyl, amides, ether compounds, thioether, amidine derivatives, guanidine derivatives, glutamyl derivatives, nitrate (ONO₂), nitro (NO₂), nitrile, trifluoromethyl (—CF₃), trifluoromethoxy (—OCF₃), O-alkyl, S-alkyl, NH-alkyl, N-dialkyl, O-aralkyl, S-aralkyl, NH-aralkyl, amino, azido (N₃), hydrazino (NHNH₂), hydroxylamino (ONH₂), sulfoxide (SO), sulfone (SO₂), sulfide (S—), disulfide (S—S), and silyl groups. Typically, spacer linkers are bifunctional, wherein the functionalities may be the same or different, for example N-hydroxysuccinimides and hydrazides. Following coupling of the binding agent to the solid carrier, excess functional groups (unreacted groups) on the solid carrier are blocked to prevent further reaction with binding agent and blood components during treatment.

In some embodiments, the appropriate amount of binding agent immobilized on the solid carrier should be sufficient to provide effective reduction of the relevant ligands in the blood or plasma fraction following passage through the solid carrier containing the immobilized binding agent. Factors to consider include, among others, the type and form of solid carrier, the density of functional groups on the carrier, the number of functional groups on the binding agent, form and effectiveness of coupling agent, the percentage of functional binding agents remaining following immobilization, steric hindrance of immobilized binding agents, and the flow rate following immobilization with binding agent. For example, N-hydroxysuccinimide (NHS) activated agarose has a capacity of about 20 to about 50 mg of IgG antibody per ml of resin, with coupling efficiencies of about 40 to 80%. Another example is cyanogen bromide activated Sepharose (crosslinked agarose), which can have capacities of about 10 to about 20 mg of antibody per ml of resin and coupling efficiencies of about 50 to over 90%. In some embodiments, the solid carrier comprises about 20 nmole to about 350 nmole of binding agent per ml of solid carrier; particularly about 30 nmole to about 300 nmole binding agent per ml of solid carrier; more particularly about 60 nmole to about 300 nmole binding agent per ml of solid carrier; about 120 nmole to about 300 nmole binding agent per ml of solid carrier, or about 120 nmole to about 200 nmole binding agent per ml of solid carrier. In immobilizing the binding agent to the solid carrier, coupling efficiencies should be balanced with the binding capacity of the resulting solid carrier adsorbent. For example, a very high coupling efficiency, e.g., greater than 95% coupling using cyanogen bromide, may result in reduction in binding activity of the binding agent, which may be due to covalent attachment at two or more functional groups on the binding agent.

In some embodiments, the general procedure for the treatment methods can be similar to methods used in conventional immunopheresis, sometimes also referred to as immunoapheresis. Generally, blood from an appropriate subject (e.g., a patient afflicted with a disease characterized by abnormal NKG2D ligand levels) is removed and treated extracorporeally using an apheresis device. The blood is separated into its cellular elements (e.g., fraction containing red blood cells, white blood cells and platelets) and fluid elements (e.g., plasma fraction) using differential centrifugation, a membrane filter or other compatible blood-plasma separator. The plasma is then pumped through the apheresis device where the circulating sNKG2D ligands will bind to the immobilized binding agent and be removed from the plasma fraction. The treated plasma fraction is then mixed with the cellular blood elements and returned to the subject, thereby removing the sNKG2D ligands from circulation. The appropriate flow rate of plasma during apheresis can be readily determined by the skilled artisan, for example by taking into consideration, among others, the blood flow rate, the type of plasma separator (e.g., filter versus centrifugal), the column material, efficiency of binding agent in binding sNKG2D ligands, and age of the subject. For example, a flow rate range of about 10 ml/min to about 100 ml/min, more particularly a range of about 30 ml/min to 100 ml/min can be used. The prescribed blood volume subject to treatment can be determined by the skilled artisan, e.g., taking into consideration the patient's size. A typical adult human weighing about 70 Kg has a blood volume of about 4.7 to about 5 liters; a typical adult male has a plasma volume of about 46 ml/Kg body weight to 52 ml/Kg body weight (see, e.g., Yiengst and Shock, 1962, J Applied Physiol. 17(2):195-8). In some embodiments, the plasma volume treated is about 20 to about 150 ml plasma/Kg body weight, more particularly about 40 to about 70 ml plasma/Kg body weight. In some embodiments, at least 1 times the total blood volume (based on approximately 7% of the total body weight), particularly at least 1.5 times the total blood volume, more particularly at least 2 times the total blood volume, or plasma equivalents thereof are processed over a defined period in a single treatment session, for example a time period of about 0.25 to about 4 hrs, about 1 to 3 hrs, particularly about 0.5 to about 1 hr, more particularly about 0.25 to about 0.5 hr. In some embodiments, about 3 to about 15 liters, particularly 3 to 10 liters, more particularly 4 to 7.5 liters of plasma is treated in a single treatment session. In some embodiments, about 2.5, 3, 4, 5, 7.5, 10, 12.5, or 15 liters of plasma is treated in a single treatment session. Typically, in some embodiments, an anticoagulant, such as heparin or citrate is used during treatment.

In some embodiments, the plasma fraction or blood is treated to remove at least 20%, at least 30%, at least 40%, at least 50%, at least 60%, at least 70%, at least 80%, at least 90% or more of sNKG2D ligands present in the blood. In some embodiments, the treatment of the blood can be performed a plurality of times. In some embodiments, the sNKG2D ligands complexed to the binding agent on the solid carrier are removed prior to each performance of the treatment.

Generally, the number, frequency and duration of treatment will depend on, among others, the characteristic of the disease or disorder, the stage of treatment (e.g., naïve patient versus previously treated), and the rate of reappearance of the sNKG2D ligand following treatment. In some embodiments, the treatments are performed at least once a week, at least once every two weeks, or at least once a month. In some embodiments, the patient's blood is treated once daily for at least 3 days, 4 days, or 5 days in a week. In some embodiments, the days are consecutive days. In some embodiments, treatment of the blood is performed once daily for at least 5 days, particularly during the initial stages of therapy where the levels of sNKG2D ligand can be relatively high. In some embodiments, the treatments can be continued as deemed appropriate by the medical practitioner. The duration of treatments can be at least two weeks, or 1, 2, 3, 4 or 6 months or more, up to one year, or up to two years. Periodic maintenance therapy can be conducted as necessary, for example, to maintain the sNKG2D ligands at levels below that which result in immunosuppression.

In some embodiments, the method of removing sNKG2D ligands from the blood can be used to treat various diseases or disorders characterized by elevated levels of sNKG2D ligand. In some embodiments, the method can be applied to a wide range of mammals, including, among others, humans, non-human primates (e.g., chimpanzees, monkeys, etc.), or non-primates (e.g., horses, cattle, pigs, sheep, deer, elk, goats, dogs, cats, rabbits, rats, and mice). Generally, the subject or patient is preferably a human In certain embodiments, the human is a pediatric patient. In other embodiments, the human is an adult patient. In some embodiments, the subject can be an ape (e.g., gorilla, chimpanzee, or orangutan) or a domesticated mammal (e.g., dog, cat, sheep, cow, or horse).

In some embodiments, the method can be used to treat a subject afflicted with a sMIC⁺ and/or sULBP⁺ tumor, hematologic malignancy, or viral infection.

In some embodiments, the method can be used to treat a subject afflicted with a sMICA³⁰ and/or sMICB⁺ tumor, hematologic malignancy, or viral infection (e.g., the elevated sNKG2D ligand comprises sMICA and/or sMICB).

In some embodiments, the disease comprises a sMIC⁺ cancer or tumor. Such exemplary tumors or cancers include, among others, brain cancer, lymphatic cancer, liver cancer, stomach cancer, testicular cancer, cervical cancer, ovarian cancer, vaginal and vulvar cancer, leukemia, melanoma, squamous cell carcinoma, malignant mesothelioma cancer, oral cancer, head and neck cancer, throat cancer, thymus cancer, gastrointestinal stromal tumor (GIST) cancer, nasopharyngeal cancer, esophageal cancer, pancreatic cancer, colon cancer, anal cancer, breast cancer, lung cancer, prostate cancer, penile cancer, bladder cancer, neuroblastoma, glioma, hepatocellular carcinoma, and renal cancer. More specifically, in some embodiments, the therapeutic method can be used for treating epithelial tumors, including but not limited to, lung, breast, gastric, colon, ovarian, renal cell, and prostate carcinomas, and melanoma.

In some embodiments, the disease or disorder comprises a sMIC⁺ hematologic malignancy. Exemplary hematologic malignancies that can be treated include, among others, Acute Lymphoblastic Leukemia (ALL), Acute Myelogenous Leukemia (AML), Chronic Lymphocytic Leukemia (CLL), Chronic Myelogenous Leukemia (CML), Acute Monocytic Leukemia (AMol); lymphomas, including Hodgkin's lymphoma, Non-Hodgkin's lymphoma, and precursor T-cell leukemia/lymphoma, follicular lymphoma, diffuse large B-cell lymphoma, mantle cell lymphoma, MALT lymphoma, Burkitt's lymphoma, B-cell chronic lymphocytic leukemia/lymphoma, peripheral T-cell lymphoma—not-otherwise-specified, and mycosis fungoides; and Multiple Myelomas.

In some embodiments, the disease or disorder comprises a sMIC⁺ viral infection. Exemplary viral infections that can be treated include, among others, infections with hepatitis-B virus (HBV), respiratory syncytial virus (RSV), human cytomegalovirus (HCMV), hepatitis c virus (HCV), and human immunodeficiency virus (HIV).

In some embodiments, the method can be used to treat a subject afflicted with a ULBP⁺ tumor, hematologic malignancy, or viral infection (e.g., the elevated soluble NKG2D ligand comprises sULBP1, sULBP2, sULBP3, sULBP4, sULBP5 and/or sULBP6).

In some embodiments, the disease or disorder characterized by elevated sULBP⁺ levels comprises a sULBP⁺ tumor. Exemplary sULBP⁺ tumors include, among others, melanoma, ovarian cancer, pancreatic cancer, malignant glioma, lung cancer, squamous cell carcinoma, and gastric cancer.

In some embodiments, the disease or disorder characterized by elevated sULBP⁺ levels comprises a sULBP⁺ hematologic malignancy. Exemplary sULBP⁺ hematologic malignancies include, among others, Myeloid Leukemia, Acute Lymphoblastic Leukemia (ALL), Acute Myelogenous Leukemia (AML), Chronic Lymphocytic Leukemia (CLL), Chronic Myelogenous Leukemia (CML), Acute Monocytic Leukemia (AMol); Hodgkin's lymphoma, Non-Hodgkin's lymphoma, and Multiple Myeloma.

In some embodiments, the disease or disorder characterized by elevated sULBP⁺ levels comprises a sULBP⁺ viral infection. Exemplary viral infections that can be treated include, among others, infections with hepatitis-B virus (HBV), respiratory syncytial virus (RSV), human cytomegalovirus (HCMV), and human immunodeficiency virus (HIV).

The method of treatment herein can be used in isolation (i.e., as monotherapy) or in combination with other treatments used to treat diseases associated with elevated levels of sNKG2D ligand. In the combination therapy, the methods of the disclosure can be used simultaneously, sequentially or separately from the treatments with other therapeutic agents or methods.

In some embodiments, the methods, systems and devices can be used in combination with chemotherapeutic agents used to treat tumors and cancers. The treatments with chemotherapeutic agents can include, among others, use of cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors. Exemplary chemotherapeutic agents typically used to treat proliferative disorders, such as cancers and tumors, include, by way of example and not limitation, afatinib, afuresertib, alectinib, alisertib, alvocidib, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, floxuridine, fludarabine, fluorouracil, gefitinib, gemcitabine, hydroxyurea, ibrutinib, icotinib, idarubicin, ifosfamide, imatinib, imetelstat, ipatasertib, irinotecan, ixabepilone, lapatinib, lenalidomide, lestaurtinib, lomustine, lucitanib, masitinib, melphalan, mercaptopurine, methotrexate, midostaurin, mitomycin, mitoxantrone, mubritinib, nelarabine, neratinib, nilotinib, nintedanib, omacetaxine mepesuccinate, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, valrubicin, vandetanib, vemurafenib (Zelborae), vincristine, vinblastine, vinorelbine, vindesine, and the like.

In some embodiments, the methods, systems and devices can be used in combination with a biologic drug used to treat tumors, cancers, and autoimmune diseases. Exemplary biologic drugs that can be used include, among others, anti-BAFF (e.g., belimumab); anti-CCR4 (e.g., mogamulizumab); anti-CD19/CD3 (e.g., blinatumomab); anti-CD20 (e.g., obinutuzumab, rituximab, ibritumomab tiuxetan, ofatumumab, tositumomab); anti-CD22 (e.g., moxetumomab pasudotox); anti-CD30 (e.g., brentuximab vedotin); anti-CD33 (e.g., gemtuzumab); anti-CD37 (e.g., otlertuzumab); anti-CD38 (e.g., daratumumab); anti-CD52 (e.g., alemtuzumab); anti-CD56 (e.g., lorvotuzumab mertansine); anti-CD74 (e.g., milatuzumab); anti-CD105; anti-CD248 (TEM1) (e.g., ontuxizumab); anti-CTLA4 (e.g., tremelimumab, ipilimumab); anti-EGFL7 (e.g., parsatuzumab); anti-EGFR (HER1/ERBB1) (e.g., panitumumab, nimotuzumab, necitumumab, cetuximab, imgatuzumab, futuximab); anti-FZD7 (e.g., vantictumab); anti-HER2 (ERBB2/neu) (e.g., margetuximab, pertuzumab, ado-trastuzumab emtansine, trastuzumab); anti-HER3 (ERBB3); anti-HGF (e.g., rilotumumab, ficlatuzumab); anti-IGF-1R (e.g., ganitumab, figitumumab, cixutumumab, dalotuzumab); anti-IGF-2R; anti-KIR (e.g., lirilumab, onartuzumab); anti-MMP9; anti-PD-1 (e.g., nivolumab, pidilizumab, lambrolizumab); anti-PD-Ll; anti-PDGFRa (e.g., ramucirumab, tovetumab); anti-PD-L2; anti-PIGF (e.g., ziv-aflibercept); anti-RANKL (e.g., denosumab); anti-TNFRSF9 (CD137/4-1BB) (e.g., urelumab); anti-TRAIL-R1/DR4,R2/D5 (e.g., dulanermin); anti-TRAIL-R1/D4 (e.g., mapatumumab); anti-TRAIL-R2/D5 (e.g., conatumumab, lexatumumab, apomab); anti-VEGFA (e.g., bevacizumab, ziv-aflibercept); anti-VEGFB (e.g., ziv-aflibercept); and anti-VEGFR2 (e.g., ramucirumab).

In particular, the methods, systems and devices described herein can be used in combination with treatments that activate the immune system. In some embodiments, these can comprise use of agents that positively activate the immune system, or agents that inhibit downregulation of immune system activation. The immune activating agents can be small molecule compounds, antibodies, anti-sense compounds, gene therapy, and the like. Various biological targets for therapeutic immune activation agents include, by way of example and not limitation, CTLA4, KIR (Killer-cell immunoglobulin-like receptor), PD-1, PD-L1, PD-L2, CD137, CD227, IL-15 receptor, IL-6, IL-6 receptor, TGF-β1, TGF-β2, TGF-β3, and apolipoprotein J (Clusterin). In some embodiments, the treatment with immune system activating agent include use of antibodies or other binding agents directed against the therapeutic targets, for example, anti-CTLA4, anti-PD-1, anti-PD-L1, anti-PD-L2, anti-CD137, anti-TGF-β1, anti-TGF-β2, anti-TGF-β3, and anti-apolipoprotein J (Clusterin). Exemplary immune activating agents include, among others, ipilimumab, tremelimumab (Ribas et al., 2013, J Clin Oncol. 31:616-22), nivolumab (Wolchok et al., 2013, N Engl J Med. 369:122-33), BMS-936559 (MDX-1105: Brahmer et al., 2012, N Engl J Med. 366:2455-65), MEDI4736 (anti-PD-L1), MPDL3280A (anti-PDL-1), lambrolizumab (Hamid et al., 2013, N Engl J Med. 369:134-44), pidilizumab (anti-PD-1; Berger Ret al., 2008, Clin Can Res. 14:3044-51), AMP-224 (PD-L2-Ig), lambrolizumab, urelumab (Li and Liu, 2013, Clin Pharmacol. 5(Suppl 1):47-53), PF-05082566 (Fisher et al., 2012, Canc. Immunol Immunother. 61:1721-33), ALT-803 (IL-15 agonist; Xu et al., 2013, Canc. Res. 73:3075-86; Zhu et al, 2009, J Immunol. 183:3598-7), AB-16B5 (anti-Clusterin), pirfenidone (Noble et al., 2011, Lancet 377:1760-69), fresolimumab (Trachtman et al., Kidney Int. 79:1236-43), sultiximab, and tocilizumab.

In some embodiments, the immune stimulating agent for use in combination with the methods, systems and devices herein can comprise a cytokine or chemokine that activates the immune response. Exemplary cytokines and chemokines include, among others, IL-2, IL-7, IL-12, IL-15, IL-21, GM-CSF, and CCL-21. In some embodiments, the immune stimulating cytokines and chemokines can be used ex vivo to treat immune cells.

In some embodiments, the methods, systems, and devices described herein can be used in combination with treatments using cancer vaccines, which includes antigen presenting cells (e.g., dendritic cells) activated with cancer vaccines. Exemplary cancer vaccines include, among others, prostatic acid phosphatase (e.g., Provenge); gp-96-Ig (e.g., HS-410); PANVAC; HER2/neu (e.g., nelipepimut-S, AVX901); DCVax(R)-L; rindopepimut; IMA950 (multi tumor associated peptides); tumor-derived heat shock protein gp96 (Vitespen); surviving peptide (e.g., ISA-51: US patent publication 20110091489); EGFRvIII-NY-ESO-1 (e.g., ADU-623); CD-133; folate binding protein vaccines E39 and J65; HLA-A2 tumor antigen peptides; carcinoembryonic antigen (CEA); universal tumor antigen oncofetal antigen/immature laminin receptor protein (OFA/iLRP); mammaglobin-A; bi-shRNAfurin; HLA-A*2402 restricted epitope peptides CDCA1, URLC10, KIF20A, DEPDC1 and MPHOSPH1; hyperglocosylated MUC1 (e.g., ONT-10); poly-ICLC; human telomerase reverse transcriptase (e.g., hTERT, UV1, GV1001); HPV P16 37-63-peptide; HPV-16-E7 (e.g., ADX11-001), pNGVL4a-Sig; Herpes Zoster vaccine GSK1437173A; NY-ESO-1 antigen; leukemia-associated antigen WT1; bcr-abl p210-b3a2 breakpoint-derived pentapeptide CMLVAX100; lung cancer cell with GM-CSF (e.g., GVAX); Wilms tumor gene 1 (WT1) peptide (e.g., OCV-501); human MUC1 antigen (e.g., L-BLP25); MUC1 peptide tecemotide; HLA-A*0201 restricted epitope peptide URLC10, VEGFR1 and/or VEGFR2 9URLC10; cancer-testis antigens (e.g., URLC10, CDCA1, KIF20A, MAGE-C1, MAGE-A3/6, etc.); autophagosome-enriched vaccine Dribble, L523S protein; RNActive derived lung cancer vaccine CV9202; CSF-470 vaccine; melanoma antigen MAGE-3.A1; melanoma antigen NA17.A2; melanoma antigen IMP321; melanoma antigen LAG-3; IBBL antigen (e.g., A2/4-1BBL) melanoma vaccine; MART-1; gp100 (e.g., g209-2M, G280-9V); KRN7000; PVX-410; PROSTVAC; peptide pyroEHWSYGLRPG (PEP223); prostate specific antigen; and PSMA antigen (e.g., BPX-201).

In some embodiments, the methods, systems, and devices can be used in combination with antiviral drugs used to treat viral infections characterized by elevated levels of sNKG2D ligands, for example, infections with Hepatitis-B Virus, Respiratory Syncytial Virus, Human Cytomegalovirus, Hepatitis-C virus, and Human Immunodeficiency Virus. Drugs for treating Hepatitis-B viral infections include, among others, interferons (e.g., interferon alpha-2b or pegylated interferon), lamivudine, adefovir dipivoxil, entecavir, telbivudine, and tenofovir. Drugs for treating Respiratory Syncytial Virus include, among others, RSV hyperimmune globulin; palivizumab; benzimidazoles BMS-433771, TMC353121 and JNJ-2408068; ribavirin; and antisense phosphorodiamidate morpholino oligomers (see review Olszewska and Openshaw, 2009, Expert Opin Emerg Drugs 14(2):207-17). Drugs for treating Hepatitis-C Virus include, among others, interferons (e.g., interferon alpha-2b or pegylated interferon), boceprevir, telaprevir, ribavirin, simeprevir, sofosbuvir, daclatasvir, and combinations thereof. Drugs for treating Human Immunodeficiency Virus include, among others, efavirenz, emtricitabine, tenofovir disoproxil fumarate, rilpivirine, cobicistat, lamivudine, zidovudine, abacavir, zalcitabine, stavudine, nevirapine, etravirine, delavirdine, tipranavir, indinavir, saquinavir mesylate, lopinavir, ritonavir, darunavir, atazanavir sulfate, nelfinavir mesylate, maraviroc, raltegravir, enfuvirtide, and combinations thereof.

In some embodiments of the combination treatment, the subject can be treated prior to, concurrently with, or subsequent to the other treatments described above. For example, for use in combination with an immune stimulating agent, a subject can be treated using the methods herein prior to treatment with an immune stimulating agent or cancer vaccine. Subsequently, a follow-up treatment to remove sMICA and/or sMICB ligands can be carried out concurrently with the treatment involving the immune stimulating agent or cancer vaccine. Other treatment schemes for the combination of the methods of the disclosure and other therapeutic agents will be apparent to the skilled artisan in light of the guidance herein.

In another aspect, the present disclosure provides a system for carrying out the therapeutic method of removing soluble NKG2D (sNKG2D) ligands. In some embodiments, the system comprises:
a plasma separator capable of separating plasma fraction from blood cell fraction;
a chamber containing a binding agent capable of specifically binding a sNKG2D ligand, wherein the binding agent is immobilized on a solid carrier; and
a pump for moving the separated plasma fraction through the chamber.

In some embodiments, the chamber is in fluid communication with the plasma separator, either directly or indirectly. For example, the chamber can be connected directly to the outlet of the plasma fraction of the plasma separator, or the chamber connected indirectly to the plasma separator through a tube or channel. Generally, the chamber and the plasma separator are in fluid communication when the system is being used to treat a subject's blood.

Various types of plasma separators can be used in the system herein. In some embodiments, the plasmid separator can comprise a centrifuge, which can be continuous flow or intermittent flow, and the separation based on differential centrifugation (see, e.g., U.S. Pat. No. 4,425,112 and U.S. Pat. No. 5,386,734; and patent publication no. US20080200859). In some embodiments, the plasma separator comprises a filter membrane, such as hollow fiber membranes (see, e.g., U.S. Pat. No. U.S. Pat. No. 4,631,130 and U.S. Pat. No. 6,802,820; Malchesky, P. S., 2001, Ther Apher. 5(4):270-82). When the plasma separator is a filter membrane, the filter is generally biocompatible and suitable for contact with blood, without causing excessive activation of platelets or clotting. In some embodiments, the plasma separator filter membranes can be either parallel plate filters or capillary membrane filters. In some embodiments, the filter membranes can be made of a biocompatible, inert thermoplastic such as polycarbonate, polytetrafluoroethylene (Teflon®), polypropylene, ethylene polyvinyl alcohol, or polysulfone. It is often desirable to profuse proteins in the lower molecular weight fraction of the plasma over the adsorber, thereby avoiding profusion of large macromolecular proteins, such as fibrinogen, alpha-2 macroglobulin and macroglobulins, such as cryoglobulins. Therefore, membranes that possess molecular sieving discrimination in these molecular sizes are desirable. In some embodiments, such membranes can have a pore size typically of between about 0.02 and about 0.05 microns in a capillary membrane filter and of between 0.04 and 0.08 microns in a parallel plate filter. The actual pore size that yields the desired cut-off can be determined based on the fluid flow geometry, shear forces, flow rates, and surface area. By way of example and not limitation, the effective cut-off for a capillary membrane filter with a pore size of 0.03 microns is about 150,000 daltons, with a sieving coefficient of between about 10 and about 30%. Generally, the permeable membrane should not cause blood clotting or otherwise react with the blood. Suitable filter devices can be obtained from Asahi Kasei Medical, and specifically the PlasmaFlo OP™ Series hollow fiber plasma separator. Another suitable filter is the Fresenius polysulfone filter. Staged filters that have different pore sizes and/or geometries or surfaces areas, to provide for a “staggered” removal of materials from the blood can also be used.

In some embodiments, the system comprises a chamber containing a binding agent capable of specifically binding a soluble NKG2D ligand, where the binding agent is immobilized on a solid carrier. The binding agent can be any agent that is capable of binding specifically to soluble NKG2D ligands, as described herein. In some embodiments, the binding agent comprises any of the antibody agents that bind specifically to soluble NKG2D ligands. These include, without limitation, antibodies that are polyclonal, monoclonal, multispecific, non-human, chimeric, humanized, fully human, or combinations thereof. The binding agents can comprise fragments of the antibodies, or single chain antibodies.

In some embodiments, the antibody binds specifically to sMICA and/or sMICB protein. In some embodiments, the antibody binds specifically to the alpha-1 domain, alpha-2 domain, and/or alpha-3 domain of the sMICA or sMICB. In some embodiments, the antibody binds specifically to the alpha-3 domain, particularly to cryptic epitopes on the alpha-3 domain that are defined by the sequences disclosed above.

In some embodiments, the antibody binds specifically to sULBP1, sULBP2, sULBP3, sULBP4, sULBP5, or sULBP6 protein. In some embodiments, the antibody binds specifically to the alpha-1 and/or alpha-2 domain of sULBP1, sULBP2, sULBP3, sULBP4, sULBP5, or sULBP6 protein.

As discussed above, in some embodiments, the binding agent can comprise a receptor of sMICA, sMICB, and/or sULBP (e.g., sULPB2, or sULPB3) protein. In some embodiments, the receptor comprises a NKG2D receptor, a functional HCMV UL16 viral protein, a functional HCMV UL142 viral protein, a functional HHV-7 U21 viral protein, or functional fragments thereof. The receptors can be used without further modification or in some embodiments, prepared as a fusion protein, for example to an Fc portion of an immunoglobulin or a peptide linker that provides flexibility and/or functional groups for immobilization on the solid carrier.

The solid carrier to which the binding agent is immobilized can be made of agarose, dextran, polyacrylamide, silica, polysulfone, cellulose, polyamide, polyether, polyethylene, polypropylene, polyester, and derivatives and mixtures thereof. Particularly useful solid carriers are crosslinked agarose, such as Sepharose. In some embodiments, the solid carrier can be in the form of beads or other particles to facilitate flow and contact with the blood or plasma. In some embodiments, the solid carrier can be in the form of hollow fibers or membranes, channels, tubes or other configurations to which the binding agent is immobilized to increase the surface area and contact with the blood or plasma. In some embodiments, the chamber containing the solid carrier can comprise a column, which may be straight, coiled or any other configuration that provides efficiency to the process. In some embodiments, the chamber comprises a disposable unit or removable cassette, that can be removed and replaced to avoid contamination and/or when the column has been exhausted. In some embodiments, the plasma separator and the chamber containing the solid carrier comprise a disposable or removable unit.

In some embodiments, the system can comprise two or more chambers (e.g., columns), where each chamber contains a solid carrier with immobilized binding agent. In some embodiments, the blood or plasma fraction is treated by passing it through a first chamber. After a sufficient volume has been treated, the flow of blood or plasma is switched to a second chamber for continued treatment of the blood or plasma fraction. The first chamber is washed to remove the bound ligand and to regenerate the solid carrier, e.g., by washing with normal sterile saline, elution with 200 mM glycine-HCl, pH 2.8, washing with normal sterile saline, then washing with phosphate buffered saline (PBS). The use of two or more chambers allows a greater volume of blood or plasma to be treated in a single treatment session.

In some embodiments, where the system comprises two or more chambers, a first chamber can comprise a first binding agent, and a second chamber can comprise a second binding agent, where the first binding agent binds specifically to a first NKG2D ligand, and the second binding agent binds specifically to a second NKG2D ligand, where the first NKG2D ligand is different from the second NKG2D ligand. The chambers can be placed in series or in parallel in the system to remove two or more different sNKG2D ligands, for example two or more of sMICA, sMICB, sULBP1, sULBP2, sULBP3, sULBP4, sULBP5, and sULBP6, in a single treatment session.

To facilitate the flow of the separated plasma fraction through the chamber containing the solid carrier, the system comprises a pump capable of moving or transporting the separated plasma fraction through the chamber. The pump can be any form sufficient for the purposes, and include peristaltic, piston, pneumatic, and hydraulic pumps, or other pumps known to those of skill in the art. Generally, the system comprises a controller, such as a microprocessor, to control the pump force, rate of flow, and time of operation. In some embodiments, the system can comprise more than one pump, for example, a first pump for transporting the blood through the plasma separator and the chamber containing the solid carrier, and a second pump for transporting the treated plasma fraction for reconstitution with the blood fraction and reinfusion into the subject. Either integrated or independent of the pump, the system can further comprise one or more components selected from a flow rate detector, pressure monitor, and air sensor.

In some embodiments, the system can further comprise a first conduit for accepting withdrawn blood from the patient, typically via a venous access, and a second conduit (e.g., a venous catheter) for returning or reinfusing blood that has been reconstituted following treatment of the plasma fraction back to the patient, typically via a second venous access. In some embodiments, the patient will typically be connected to the blood processing device using an indwelling venous catheter and standard intravenous tubing.

In some embodiments, the system also comprises a mixing chamber or reservoir, where the separated blood cell fraction is delivered and eventually reconstituted with the treated plasma fraction to be delivered back into the patient. In some embodiments, the system comprises a third, fourth or more reservoirs for storing reagents for carrying out the therapeutic methods, for example, a reservoir for anticoagulants (e.g., sodium heparin or citrate dextrose), to be used during treatment of the blood; a reservoir for electrolytes that can be added to compensate for loss of such components from the blood during the procedure; and a reservoir of regenerating solution for removing bound sNKG2D ligand from the binding agents on the solid carrier. By way of example and not limitation, the solid carriers may be regenerated by washing with normal sterile saline, elution with 200 mM glycine-HCl, pH 2.8, washing with normal sterile saline, then washing with phosphate buffered saline (PBS). Other equivalent washing solutions and procedures can be used. In some embodiments, the chamber containing the solid carrier is flushed with multiple (two or more) volumes of sterile wash solution prior to use.

In some embodiments, the system for carrying out the therapeutic methods herein comprises an apheresis system. Such systems are well known in the art and can be easily adapted to all the embodiments disclosed herein. Exemplary apheresis systems are described in, for example, U.S. Pat. No. 5,112,298; U.S. Pat. No. 5,476,715; U.S. Pat. No. 5,817,528; U.S. Pat. No. 6,036,614; U.S. Pat. No. 6,565,806; U.S. Pat. No. 6,620,382; U.S. Pat. No. 7,267,771; and U.S. Pat. No. 8,317,737; all publications incorporated herein by reference. Additional devices are described in patent publications US20020119147; US20020107460; US20070026029; US20110033463; and PCT publications WO2006014646 and WO99/61095; all of which are incorporated herein by reference. Commercial apheresis systems that can be adapted to the present system include, among others, those manufactured by Fresenius, Affinia, Plasmaselect, Asahi Kasei Medical Co., Kaneka, B. Braun, etc. In some embodiments, apheresis devices useful for the purposes herein, include, among others, LDL-R Therasorb™; Immunosorba™; Prosorba™; Globaffin™; Ig-Therasorb™; Immusorba™ TR-350 (L); Immusorba™ PH-350 (L); Liposorba™; HELP™; DALI™; bilirubin-bile acid Absorber BR-350; Plasorba™ BR-350 (L); Prometheus™ detoxification; MARS™; ADAsorb system of Medicap or Plasma-flo™ system; Baxter Healthcare CS3000; Amicus centrifugal apheresis and Autopheresis C; Terumo BCT (COBE Spectra and Spectra OPTIA apheresis system); and Haemonetics Corporation (PCS 2™). All these systems can be fitted by an apheresis specialist, for example, as immunopheresis and/or by installation of the solid support disclosed herein.

In view of the descriptions above, in another aspect, the present disclosure is directed to an apheresis device for treating a subject afflicted with a disease characterized by abnormal (e.g., elevated) levels of sNKG2D ligand. In some embodiments, the device comprises a solid carrier capable of being contacted with flowing blood or plasma, wherein the solid carrier comprises a binding agent that binds specifically to a sNKG2D ligand or two or more sNKG2D ligands.

In some embodiments of the device, the binding agent comprises any of the antibodies described herein, or alternatively a receptor that is capable of binding specifically to sNKG2D ligand (e.g., soluble ligand sMICA, sMICB, sULPB1, sULPB2, sULPB3, sULPB4, sULPB5 and sULPB6). As described in the present disclosure, the receptor can comprise one or more of a NKG2D receptor, HCMV UL16 viral protein, HCMV UL142 viral protein, HHV-7 U21 viral protein, or functional fragments thereof that bind specifically to the soluble NKG2D ligand. Accordingly, it is to be understood that all embodiments of binding agents of the present disclosure are to be included in the embodiments of the device.

In some embodiments of the device, the binding agent is immobilized on a solid carrier. Any of the solid carrier materials, including agarose, dextran, polyacrylamide, silica, polysulfone, cellulose, polyamide, polyether, polyethylene, polypropylene, polyester, and derivatives and mixtures thereof, can be used. An exemplary solid carrier for use in the device is a crosslinked agarose. The solid carriers can be in any suitable form, such as beads or particles, uniform or non-uniform, or in the form of tubes or channels. In some embodiments, the solid carriers comprise an inert polymeric matrix, such as SEPHAROSE™, manufactured by Amersham-Biosciences, Uppsala, Sweden, within a medical grade housing, e.g., polycarbonate. Standard techniques for coupling of antibodies to the gel material can be used, as described above. Any of the characteristics of the solid carriers as described herein for the method and systems are applicable to the device.

In some embodiments, the solid carriers comprise filter membranes or capillary dialysis tubing to which the binding agents are immobilized and where the plasma passes adjacent to, or through the membranes. Suitable filters include those discussed above with respect to separation of blood components. These may be the same filters, having immobilized binding agents bound thereto, or may be arranged in sequence, so that the initial filter separates the blood components and the subsequent filter removes the soluble NKG2D ligand.

In some embodiments of the device, the solid carrier is contained in a chamber, for example, a column. In some embodiments, the device can comprise two or more chambers, which can be present in series or in parallel, as described in the present disclosure for the systems. In some embodiments, the chambers can contain the same binding agent, or in some embodiments, different binding agents.

The solid carrier with the immobilized binding agent can be packed into the column after sterilization or aseptic treatment of the material. In some embodiments, coupling of the binding agent, such as an antibody, to the matrix using a technique such as cyanogen bromide can significantly reduce contamination of the solid carrier. For example, virus contamination in an antibody preparation can be reduced either by removal of the unbound virus during washing or by coupling the virus to the solid carrier (e.g., matrix material) during immobilization of the binding agent to the solid carrier, which inactivates the bound virus. In some embodiments, following immobilization of the binding agent to the solid carrier, the material can be sterilized by irradiation, for example, by spreading the solid carrier material in a bag to maximize its exposed surface area and irradiation with stationary e-beam radiation (e.g., 24 centi). Other known sterilization techniques that may be used, alone or in combination, include washing the matrix material containing immobilized binding agent with glycine at a pH of 2.8, ultraviolet irradiation, ethylene-oxide saturation, glutaraldehyde saturation, gamma-irradiation, and/or detergent treatment. The sterilized or aseptically prepared solid carrier or matrix material can be transferred into a sterilized column, for example a sterile port in the bag that connects directly to a port of the column.

Column housings can be sterilized prior to packing with the solid carrier containing the immobilized binding agent. Columns can be filled with 0.1% sodium azide in phosphate buffered saline (“PBS”) as a preservative, although other medically equivalent buffers could be used. The packed columns can be stored refrigerated until use.

As described above, in some embodiments of the device, the chamber, such as a column, can comprise a disposable unit or removable cassette, that can be removed following use and/or replaced to avoid contamination and/or when the column has been exhausted. In some embodiments, the chamber containing the solid carrier comprises a disposable or removable unit.

In some embodiments, the chamber can be employed as a single use device or it may be regenerated and used multiple times. For example, to regenerate the device an elution buffer solution, such as glycine-HC1, pH 2.8, is passed through the device to release the sNKG2D ligand bound to the immobilized binding agent. The released sNKG2D ligands are washed out of the device and the regenerated column matrix is then washed and stored in physiological buffer, such as phosphate buffered saline pH 7.2 with preservatives. Other similar elution buffers and storage buffers are known to those skilled in the art and are within the scope of this disclosure. Typically, the cartridge device is stored at 2-8° C.

In some embodiments, the present method can be performed by passing the blood or plasma fraction of a patient through a device which is worn by the subject throughout the treatment period, either extracorporeally or as an implant (see, e.g., U.S. patent publication nos. 20120323158 and 20100272772, incorporated herein by reference). The device can comprise an inlet conduit, an outlet conduit, and a chamber containing the solid carrier immobilized with the relevant binding agent. The device can comprise a semi-permeable polymeric or biopolymeric membrane which excludes cells and cellular components and allows primarily blood plasma to pass through to the inner part of the chamber containing the immobilized binding agent. In some embodiments, blood or other body fluid of a subject can be directed through the chamber of the device using a blood pump. A number of blood pumps are known to the art, including pumps designed to be implanted, such as the pump disclosed in U.S. Pat. No. 6,641,612, incorporated herein by reference. The pump can be placed in-line with the flow of blood or other fluid through the device, either upstream or downstream from the cassette. In some embodiments, the semi-permeable polymeric or biopolymeric membrane is formed to have an inner cavity, where the binding agent is immobilized. Filtered plasma that enters the inner cavity is exposed to the immobilized binding agent and the flows out through an outlet port. Thus, the plasma filter membrane and the solid carrier with immobilized binding agent comprise a single filter membrane.

In another aspect, further provided are kits for use in the methods, systems and devices. In some embodiments, the kit comprises a solid carrier, wherein the solid carrier is immobilized with a binding agent that binds specifically to a sNKG2D ligand. In some embodiments, the binding agent binds specifically to one or more of sMICA, sMICB, sULBP1, sULBP2, sULBP3, sULBP4, sULBP5, and sULBP6 proteins. Any of the binding agents that bind to a sNKG2D ligand can be immobilized on the solid carrier, for example, antibody that binds specifically to the sNKG2D ligand.

In some embodiments, the kit comprises a chamber, such as a column, wherein the chamber comprises a solid carrier with an immobilized sNKG2D-ligand binding agent. The solid carrier in the chamber can be in the form of particles or tubes (e.g., hollow fibers). The solid carrier can be selected from agarose, dextran, polyacrylamide, silica, polysulfone, cellulose, polyamide, polyether, polyethylene, polypropylene, polyester, and derivatives (e.g., crosslinked) and mixtures thereof. In some embodiments, the chamber is configured to be used in a system described above, for example an apheresis system. In such embodiments, the chamber is configured into a cassette that can be inserted and removed for use in the system.

In some embodiments, the kits can further comprise solutions, or components for preparing solutions, for washing, regenerating, and/or sterilizing the solid carrier/chamber, for example saline, phosphate buffered saline, glycine-HCl (pH 7.2), and preservative solution (e.g., sodium azide in PBS)

In some embodiments, the kits can also comprise components for determining the levels of sNKG2D ligand, for example a primary antibody that binds specifically to sMIC and/or sULBP protein. In some embodiments, the primary antibody contains a label that can be detected. In some embodiments, the assay kit can include components for detecting the antibody specific to sMIC and/or sULBP protein, for example a secondary antibody or binding agent that binds to the primary antibody. The secondary or primary antibody-specific binding agent can contain a label, such as a fluorescent, enzymatic, or chemiluminescent label. The assay components can be used to ascertain the effectiveness of the binding agent-solid carrier in removing the sNKG2D ligand from the blood of a subject.

In some embodiments, the kits can further comprise instructions for use of the solid carrier and/or chamber in the method, system, and/or device of the present disclosure. These instructions may be present in the kit in a variety of forms, one or more of which may be included in the kit. One form in which these instructions may be present is as printed information on a suitable medium or substrate, e.g., paper on which the information is printed, in the packaging of the kit, in a package insert, etc. Yet another means would be a computer readable medium, e.g., diskette, CD, computer memory, etc., on which the information has been recorded. In some embodiments, the instructions may be accessible on a website address which may be used via the internet to access the information at a remote site. Any inconvenient means for presenting the instructions can be used in the kits.

Having now generally described the invention, the same will be more readily understood through reference to the following examples which are provided by way of illustration, and are not intended to be limiting of the present invention, unless specified.

### EXAMPLES

### Example 1

### Treatment of Cancer Patients with Immunopheresis Using Anti-MICA and/or Anti-MICB Antibodies

The purpose of this study is to assess the efficacy and safety of an immunopheresis column capable of reducing circulating sMICA and/or MICB in the plasma of cancer patients.

The immunopheresis column is a sterile immune adsorbent product designed to remove soluble MICA and/or MICB or sULBP, such as sULBP2 or sULBP3, from the blood. It is designed to be used in conjunction with commercially available approved extracorporeal blood treatment systems, (e.g. Diapact CRRT device, B. Braun, Fresenius Hemocare Apheresis, Excorim Immuoadsorption Systems).

The column housing can be a 325 ml volume medical grade polycarbonate device (PNS-400146-Fresenius HemoCare, Inc.). The column matrix is composed of Sepharose 4B beads and antibodies against sMICA and/or sMICB or a sULBP (e.g., sULBP2 and sULBP3), such as those described in U.S. Pat. Nos. 7,771,718 and 8,182,809, or commercially available from various suppliers cited in the specification. The essential components for manufacturing are Sepharose, purchased as sterile product from Amersham-Biosciences (Uppsala, Sweden), antibodies to soluble MICA and/or MICB or a sULBP (e.g., sULBP2 or sULBP3) that are sterilized by filtration (Eurogentec, Liege, Belgium), and a polycarbonate housing (Fresenius), sterilized by autoclaving. Coupling of the antibodies can be carried out using standard coupling procedures, for example, cyanogen bromide activation, followed by blocking of unreacted functional groups. The extent of antibody coupling can be tested prior to use. Each column is individually tested for sterility and endotoxin level post manufacture. Each column is filled with 0.1% sodium azide in PBS and maintained between 4-8° C. prior to clinical use.

The intended purpose of the device is to serve as an adsorption column in clinical apheresis procedures. The column is part of an extracorporeal circuit using a standard plasma perfusion machine that removes blood from patients, separates the plasma by filtration, passes the filtered plasma through an adsorption column and then returns the combined plasma and cell fractions to the patient in a continuous loop system.

Indications for use of the device are disease conditions where patients may have a clinical benefit from removal of soluble MICA and/or MICB or a soluble ULBP, such as sULBP2 and sULBP3. The primary objective of this study is to lower plasma levels of relevant sNGK2D ligand to the lower end of the normal range (e.g., range of 30 pg/ml to 90 pg/ml in citrate plasma for MICA and MICB) during the procedure. The amount of plasma that is to be processed to achieve this level of reduction can be empirically derived for each patient but is estimated to be an amount of plasma roughly equivalent to one extracellular water volume. This can be calculated using body mass (approximately 20% of body mass in kilograms expressed in liters).

The secondary objective is to describe all clinical effects resulting from immunoadsorption (IA) in patients with metastatic cancer using the B. Braun Diapact plasma profusion system with the immunoaffinity column inserted into the plasma circuit. Another secondary objective is to specifically collect subjective and objective evidence of tumor inflammation and tumor necrosis and/or resolution as measured by CAT scan, NMR, and or bone scans or X-rays of osseus metastatic lesions of visceral tumors, or direct measurement of surface tumors.

The foregoing descriptions of specific embodiments of the present invention have been presented for purposes of illustration and description. They are not intended to be exhaustive or to limit the invention to the precise forms disclosed, and many modifications and variations are possible in light of the above teaching.

All patents, patent applications, publications, and references cited herein are expressly incorporated by reference to the same extent as if each individual publication or patent application was specifically and individually indicated to be incorporated by reference.

1-60. (canceled)

61. A system for removing a soluble NKG2D (sNKG2D) ligand from blood of a subj ect, comprising:
(a) a plasma separator capable of separating plasma fraction from blood cell fraction;
(b) a chamber containing a binding agent capable of specifically binding a sNKG2D ligand, wherein the binding agent is immobilized on a solid carrier; and
(c) a pump for moving the separated plasma fraction through the chamber.

62. The system of claim 61, wherein the chamber is in fluid communication with the plasma separator.

63. The system of claim 61, wherein the plasma separator comprises a filter membrane.

64. The system of claim 61, wherein the binding agent comprises an antibody that binds specifically to the NKG2D ligand.

65. The system of claim 64, wherein the antibody binds specifically to soluble MICA (sMICA) and/or soluble MICB (sMICB).

66. The system of claim 65, wherein the antibody binds specifically to the alpha-1 domain, alpha-2 domain, and/or alpha-3 domain of the sMICA and/or sMICB.

67. The system of claim 64, wherein the antibody binds specifically to a soluble ULBP (sULBP) protein.

68. The system of claim 67, wherein the antibody binds specifically to a soluble ULBP1 (sULBP1), soluble ULBP2 (sULBP2), soluble ULBP3 (sULBP3), soluble ULPB4 (sULBP4), soluble ULPB5 (sULBP5), and/or soluble ULPB6 (sULBP6)

69. The system of claims 61, wherein the solid carrier comprises agarose, dextran, polyacrylamide, silica, polysulfone, cellulose, polyamide, polyether, polyethylene, polypropylene, polyester, or derivatives or mixtures thereof.

70. The system of claims 61, wherein the chamber comprises a column.

71. An apheresis device for treating a subject afflicted with a disease characterized by elevated levels of a soluble NKG2D ligand, comprising a solid carrier capable of being contacted with flowing blood or plasma, wherein the solid carrier comprises a binding agent that binds specifically to a soluble NKG2D ligand.

72. The system of claim 71, wherein the binding agent comprises an antibody that binds specifically to the NKG2D ligand.

73. The system of claim 72, wherein the antibody binds specifically to soluble MICA (sMICA) and/or soluble MICB (sMICB).

74. The system of claim 73, wherein the antibody binds specifically to the alpha-1 domain, alpha-2 domain, and/or alpha-3 domain of the sMICA and/or sMICB.

75. The system of claim 72, wherein the antibody binds specifically to a soluble ULBP (sULBP) protein.

76. The system of claim 75, wherein the antibody binds specifically to a soluble ULBP1 (sULBP1), soluble ULBP2 (sULBP2), soluble ULBP3 (sULBP3), soluble ULPB4 (sULBP4), soluble ULPB5 (sULBP5), and/or soluble ULPB6 (sULBP6)

77. The device of claim 70, wherein the solid carrier comprises agarose, sepharose, dextran, polyacrylamide, silica, polysulfone, cellulose, polyamide, polyether, polyethylene, polypropylene, polyester, or derivatives or mixtures thereof.

78. The device of claim 77, wherein the solid carrier is contained in a chamber.

79. The device of claim 78, wherein the chamber comprises a column.
